(19) United States
(12) Patent Application Publication
(10) Pub. No.: US 2026/0355380 A1
(43) Pub. Date: Nov. 12, 2026

(54) STIMULI-RESPONSIVE MEDICAL ADHESIVE COMPOSITIONS, ARTICLES, AND METHODS

(71) Applicant: NUCEPTIVE LABS, INC., Boston, MA (US)

(72) Inventors: Michael Keith HEARON, II, Boston, MA (US); David F. LARSON, Boston, MA (US); Mark W. GRINSTAFF, Boston, MA (US); Danielle FITZGERALD, Boston, MA (US)

(21) Appl. No.: 19/123,970

(22) PCT Filed: Oct. 26, 2023
(86) PCT No.: PCT/US23/77976

§ 371 Date: Apr. 24, 2025

Related U.S. Application Data
(60) Provisional application No. 63/381,071, filed on Oct. 26, 2022.
(60) Provisional application No. 63/381,653, filed on Oct. 31, 2022.
(60) Provisional application No. 63/493,761, filed on Apr. 2, 2023.
(60) Provisional application No. 63/493,762, filed on Apr. 2, 2023.
(60) Provisional application No. 63/501,237, filed on May 10, 2023.
(60) Provisional application No. 63/501,238, filed on May 10, 2023.

(51) Int. Cl. A61L 15/58; A61F 6/04; A61L 24/00; A61L 24/06; A61L 31/10; A61L 31/14; C08F 220/18; C08F 265/06; C09J 4/06; C09J 133/10

(52) U.S. Cl. CPC A61L 15/58; A61F 6/04; A61L 24/001; A61L 24/06; A61L 31/10; A61L 31/14; C08F 220/1812; C08F 265/06; C09J 4/06; C09J 133/10; A61F 2006/043; A61F 2006/047

(57) ABSTRACT

The present disclosure relates to medical adhesive compositions containing a stimuli-responsive polymer that readily delaminates without pain following application of a stimulus. The present disclosure also relates to articles comprising the medical adhesive compositions. Methods of applying and removing the medical compositions and articles, as well as methods of making the medical adhesive compositions and articles, are also provided.

### 1. BACKGROUND

Adhesives and articles comprising the same have a variety of uses, including medical, industrial and consumer uses. Medical adhesives, in particular, can be used for tissue-tissue and tissue-medical device (e.g., catheter) bonding. They can also be used to promote healing, including by preventing egress of undesirable materials (e.g., bacteria, fluids) or absorbing fluids, or in some cases, by delivering one or more therapeutic agents.

Commercially available medical adhesives generally include: (i) fibrin-based adhesives and (ii) cyanoacrylate adhesives. Commercially available medical bandages include those coated with an acrylic adhesive. They can also be used to promote healing, including by preventing egress of undesirable materials (e.g., bacteria, fluids) or absorbing fluids, or in some cases, by delivering one or more therapeutic agents.

Commercially available adhesives for general, industrial, consumer and other use lack the ability to be removed from a surface after application in a facile manner. Commercially available adhesives for general, industrial, consumer and other use often are limited by elevated temperature performance, limited tack profile to multiple sources, limited hydrophobicity/ability to maintain performance in the presence of water, limited ability to act as a sealant and adhesive simultaneously, and limited ability to dissipate energy, resulting in higher system failure and a need for better adhesives for general, industrial consumer and other uses in addition to medical uses.

Despite their widespread use, the development and use of adhesives often involves a tradeoff between adhesive strength and use case experience (i.e., pain in removal, failure of adhesive to perform a desired use case-based performance). Known adhesives can also cause mechanical damage to tissue upon removal, resulting in pain and the potential for delayed healing.

Some populations are more at risk for mechanical damage to tissue associated with the use of medical adhesives, including age extremes (e.g., the elderly, pediatric patients), co-morbidities (e.g., diabetes, vascular disease, kidney failure), dermatological conditions and the female sex, among others. Medical Adhesive Related Skin Injury (“MARSI”) is a recognized disorder defined as any alteration in skin integrity characterized by erythema and/or other skin damage including skin tears, erosion, bulla, or vesicle that persists for 30 minutes or more after removal of a medical device containing adhesive. McNichol, L. et al. Orthopaedic Nursing 32.5 (2013): 267-281. It is associated with patient pain and discomfort generated in patients, additional costs to the health system, and extended length of hospitalization.

There remains a need for improved adhesives, including, but not limited to medical adhesives (e.g., adhesives for general, industrial and consumer use), and in particular adhesives that provide an improved use case experience, such as medical adhesives that provide strong adhesive properties and biocompatibility, as well as permit ease of removal.

### 2. SUMMARY

In one aspect, the present disclosure provides a medical adhesive composition comprising a stimuli-responsive polymer formed from one or more monomers and optionally one or more polyfunctional crosslinkers that crosslink the monomers, wherein the composition is suitable for adhering to a bodily surface, and the composition delaminates from the bodily surface to which it is adhered following application of a stimulus to the composition.

In another aspect, the present disclosure provides an article comprising the medical adhesive composition described herein adhered to a substrate.

In another aspect, the present disclosure provides a package comprising a medical adhesive composition described herein or an article described herein.

In another aspect, the present disclosure provides a kit comprising (a) a medical adhesive composition described herein or an article described herein and (b) instructions for use.

In another aspect, the present disclosure provides a method of closing a wound comprising applying a medical adhesive composition described herein to a wound having a first surface and a second surface separated by a wound cavity, wherein the composition is applied into the wound cavity; drawing the first surface and second surfaces of the wound together; and applying pressure sufficient to close the wound, whereby the composition adheres the first surface and second surface together.

In another aspect, the present disclosure provides a method of closing a wound comprising a first surface and a second surface separated by a wound cavity, the method comprising drawing the first surface and second surface together to form a narrowed wound cavity, applying a medical adhesive composition described herein to the narrowed wound cavity, and applying pressure sufficient to close the wound, whereby the composition adheres the first surface and second surface together.

In another aspect, the present disclosure provides a method of applying an adhesive article to a subject comprising applying the adhesive surface of an article described herein to a wound or bodily surface of a subject. In some embodiments, the method further comprises removing the adhesive article after at least 1 second, e.g., 1 hour, 12 hours, 24 hours, 1 day, 1 or 1 week, wherein removal comprises application of a stimulus to the article and (a) causes minimal or no pain in the subject as measured by the Wong-Baker Qualitative Pain Assessment (WBQPA), e.g., a WBQPA score of less than 4, less than 3, less than 2, less than 1, or 0 or (b) result in medical adhesive-related skin injury (MARSI).

In another aspect, the present disclosure provides a method of preparing the medical adhesive composition described herein comprising curing one or more monomers and a first amount of one or more polyfunctional crosslinkers to form a prepolymer, and post-curing the prepolymer, thereby providing the medical adhesive composition

In another aspect, the present disclosure provides method of preparing an article described herein comprising coating a prepolymer solution onto a substrate to provide a coated substrate, the prepolymer solution comprising one or more monomers and optionally one or more polyfunctional crosslinkers; curing the coated substrate; and post-curing the coated substrate thereby providing the article.

### 3. BRIEF DESCRIPTION OF THE SEVERAL VIEWS OF THE DRAWINGS

These and other features, aspects, and advantages of the provided disclosure will become better understood with regard to the following description, and accompanying drawings, where:

FIG. 1A-B shows (A) a medical adhesive comprised of a barrier layer or backing layer (0101) and an adhesive layer (0102) and (B) a medical adhesive viewed from the underside of (A) showing various configurations of adhesive patterns, such as a band or ribbon of adhesive (0111) which may be circular, semi-circular, rectangular in cross-section, and adhesive dots (0112) which may be spherical, hemi-spherical, cylindrical, rectangular, triangular, arbitrary, or random in shape.

FIG. 2 shows a plot of adhesive strength of adhesives according to Example 10.

FIG. 3 shows a graph of tensile strain capacity of adhesives according to Example 11.

FIG. 4 shows a graph of tensile strain capacity of adhesives according to Example 11.

FIG. 5 shows a graph of tensile strain capacity of adhesives according to Example 11.

FIG. 6 shows a graph of peel strength of an adhesive according to Example 12 in a 180-degree peel test using a human skin substrate analog.

FIG. 7 shows a graph of peel strength of adhesives according to Example 12 in a 180-degree peel test using a glass substrate.

FIG. 8 shows a plot of tan(δ) for adhesives according to Example 13.

FIG. 9 shows a plot of storage and loss modulus for adhesives according to Example 13.

FIG. 10 shows a zoomed-in view of loss and storage moduli for L6 adhesive illustrating crossover between elastic and viscous regimes between at 0.05 MPA and 8 rad/sec.

FIG. 11 shows a plot of the change in tan(δ) for adhesives according to Example 13 over the range of 0 °C to 50 °C.

FIG. 12 shows DMA measurements of storage (G′) and loss (G″) moduli as a function of temperature for the L6 adhesive.

FIG. 13 shows DMA measurements of tan(δ), the ratio of the loss modulus over the storage modulus, for the L6 adhesive.

FIG. 14 shows DMA measurements of storage (G′) and loss (G″) moduli as a function of temperature for the L6 adhesive.

FIG. 15 shows DMA measurements of tan(δ), the ratio of the loss modulus over the storage modulus, for the L6 adhesive.

FIG. 16 shows DMA measurements of storage (G′) and loss (G″) moduli as a function of temperature for LMA and BA adhesives.

FIG. 17 shows DMA measurements of tan(δ), the ratio of the loss modulus over the storage modulus, for LMA and BA adhesives.

FIG. 18 shows the angular strain of the L6 adhesive at different temperatures.

FIG. 19 shows the shear rate of the L6 adhesive at different temperatures.

FIG. 20 shows the angular strain of the LMA and BA adhesives at different temperatures.

FIG. 21 shows the shear rate of the LMA and BA adhesives at different temperatures.

FIG. 22 shows the tack strength of the L6 adhesive at varied different temperatures.

FIG. 23 shows the tack strength of LMA and BA adhesives at different temperatures.

FIG. 24 shows the gel fraction of the sol-gel analysis in Example 18 of various adhesives.

FIG. 25 shows a Wong-Baker qualitative pain assessment for removing various condoms containing the medical adhesive composition.

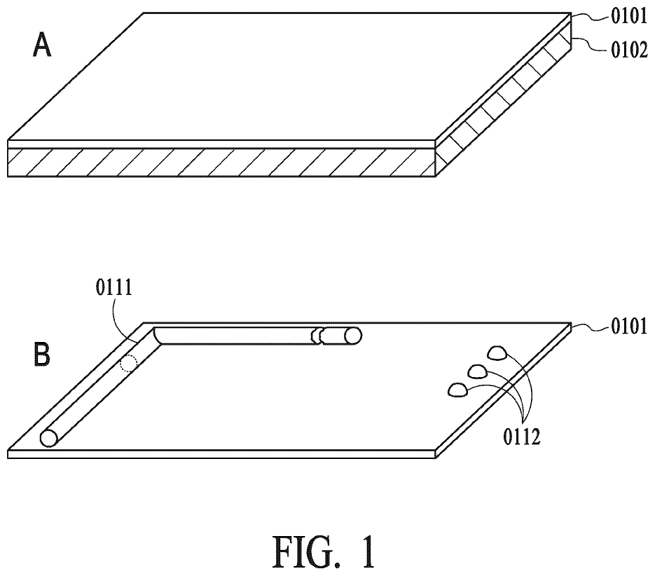

### 4. DETAILED DESCRIPTION

### 4.1. Definitions

When describing the embodiments of the present disclosure, the following terms, if present, have the following meanings, unless otherwise indicated. If not otherwise defined, terms have their customary meaning in the relevant art.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

“Adhesion,” as used herein, refers to the ability of a composition or material to adhere or “stick” to a substrate (e.g., the skin). Adhesion is measured as adhesive force in Newtons (N). The higher the adhesive force, the higher will be the number of Newtons required to peel one object from the other. With respect to bodily pathology, adhesion refers to tissue (e.g., connective tissue) that forms between adjacent injured areas within the body.

“Adhesion energy,” as used herein, refers to the amount of energy required to debond an adhesive. It can be measured by any suitable method including, without limitation, a lap sheer test. See ASTM F2255.

“Adhesive,” as used herein, refers to a composition or material that adheres to a substrate (e.g., the skin or a substrate). The adhesives described herein are suitable for many end uses, including medical adhesives, industrial adhesives and other consumer adhesives, either alone or as one component of a article. Unlike a medical adhesive, an industrial adhesive is intended for adhesion to a surface other than a bodily surface, e.g., a glass, ceramic, plastic, composite or metal surface, such as aluminum, copper, gold, nickel, silicone, silver, stainless steel alloys, and titanium. Plastic surfaces include polypropylene, polyethylene, polycarbonate, polyolefins, polyacrylics, poly(meth)acrylics, poly cyanate esters, thermosetting polymers for semiconductor use, melamine/formaldehyde substrates.

“Annulus,” as used herein, refers to a geometry defined as the region between (i) two concentric circles, or (ii) two concentric curvilinear or polygonal shapes or (iii) two approximately-concentric curvilinear or polygonal shapes.

“Anti-adhesive,” as used herein, refers to a device that reduces or eliminates undesirable adhesion of damages tissue to adjacent or surrounding tissue, generally by creating a physical barrier for a period of time in which adhesion would otherwise be expected.

“Article,” as used herein, refers to an end-use article that comprises, as an element thereof, the adhesive composition described herein and a substrate.

“Backing layer,” as used herein, refers to a layer that represents the surface layer after the application of the adhesive membrane or film to a surface. The backing layer can be removable or nonremovable.

“Bio-based,” as used herein, refers to materials derived from natural sources.

“Biodegradable,” as used herein, means used herein with reference to a composition or that can be degraded by living microorganisms like fungi or bacteria, without regard to a particular time frame or degradable by environmental conditions suitable for life such as ambient moisture and atmospheric conditions.

“Cohesion” as used herein refers to the tendency of similar or identical particles or surfaces to cling to one another.

“Compostable,” as used herein, means refers to a composition or article that require microorganisms, humidity, and heat to yield a finished compost product (CO2, water, inorganic compounds, and biomass). Compostable is distinguished from biodegradable in that compostable compositions and articles must break down into natural elements within a specific time frame. In one embodiment, the compositions and articles disclosed herein complies set forth by the U.S. Composting Council, Environmental Protection Agency, American Society for Testing and Materials (ASTM International), or Tuv Austria.

“Controlled porosity,” as used herein, refers to pores which are normally closed that are stimulated to open, thereby allowing the passage of fluids as allowed by their rheological properties.

“Crosslink Density,” as used herein, refers to average molecular weight between crosslinks. (https; www.pcimag.com/articles/104955-calculation-of-crosslink-density-of-thermoset-polymers). Flory, et al., put forth theory in crosslink density in the 1940s.

“Curing,” as used herein, refers to the chemical process of converting a macromolecule into a higher molecular weight polymer via crosslinking reactions.

“Debonding,” as used herein, refers the mechanism of debonding may vary and include, for example, phase changes, chemical reactions, crosslinking and volumetric expansion.

“Enhance” or “enhanced,” as used herein, refers to an increase in a particular property. The enhancement may be, for example, at least about 10%, at least about 20%, at least about 30%, at least about 40%, at least about 50%, at least about 60%, at least about 70%, at least about 80%, at least about 90%, or at least about 100% or more. The increase can be measured by any suitable means know in the art.

“Elastomeric” or “elastomeric behavior,” as used herein, refers to generally linear elastic or combined linear elastic and plastic deformation stress/strain behavior for a material as it is strained in a regime in a region above a key transition regime such that stress/strain hysteresis is generally conserved.

“Flexible” or “flexible behavior,” as used herein, refers to behavior of a rigid, viscoelastic or elastomeric material that can be described as compliant or deformable to fit the demands of a specific engineering application.

“Fluid,” as used herein, refers to aqueous physiological fluids, including, for example, blood, saliva, gastrointestinal fluid, lymphatic fluid, cerebrospinal fluid, gastrointestinal fluid, and mucus.

“Force responsive,” as used herein, refers to behavioral changes of a material (e.g., gel, solid, or liquid) under the application of different magnitudes of force.

“Frequency responsive,” as used herein, refers to the behavior of a material or fluid that changes under the application of a periodic or near-periodic force or displacement in time. This applied quantity can be called a “signal”. The applied signal has a defined amplitude and frequency, and optionally phase, all of which may be constant or varying in time and/or space.

“Gel Fraction” refers to the mass of a polymer remaining after washing with suitable solvent divided by initial mass. Means for determining the gel fraction are known in the art.

“Heterogeneous network,” as used herein, refers to a polymer network with a non-uniform crosslink density distribution.

“Heterogeneous crosslinking,” as used herein, refers to crosslinking distributed in a polymer network or system that is non-uniform.

“Inflammation,” as used herein, refers to a biological response of a tissue to a harmful stimulus. Common signs of inflammation include pain, heat, redness (erythema), swelling (edema), and loss of function.

“Implant,” as used herein, refers to an article or device that is placed entirely or partially inside of a subject.

“Interpenetrating network” (“IPN”), as used herein, refers to a unique type of polymer material that comprises two or more independent polymer networks, which are physically entangled but not covalently bonded to each other. Each network maintains its distinct identity, but the networks are intertwined at the molecular level, forming a composite material with properties derived from the combination of the individual networks.

“Local anesthetic agent,” as used herein, refers to an agent reduces or eliminates the sensation of pain in a specific area of the body, e.g., the skin. In one embodiment, the local anesthetic is a topical anesthetic known in the art, such as lidocaine, proparacaine, or oxybuprocaine.

“Medical adhesive”, “medical adhesive composition,” and “surgical adhesive” are used interchangeably herein to refer to an adhesive suitable for medical or surgical purposes. Representative, non-limiting commercially available medical adhesives include TEGADERM™ (3M Company, St. Paul, Minn.), Bioclous™ (Johnson & Johnson Company), Brunswick, NJ), OP-SITE™ (TJ Smith & Nephew. UK, Full) and UNIFLEX™ (Homedica).

“Medical adhesive related tissue injury” (“MARSI”), as used herein, refers to an injury that occurs when the attachment between the adhesive and the tissue is stronger than between individual cells. The injury results from the attachment between the adhesive and the skin being stronger than between the individual cells causing either the epidermal layers to separate or the epidermis to detach completely from the dermis (mechanical trauma).

“Membrane,” as used herein, refers to a layer or film of a solid, continuous polymer material.

Monomer reactivity ratios,” as used herein, refers to parameters used in polymer chemistry to describe the relative reactivity of two monomers in a copolymerization reaction. A copolymerization reaction involves the simultaneous polymerization of two different monomers, forming a copolymer with varying monomer compositions.

“Network,” as used herein with reference to a polymer, refers to macromolecular architecture formed by crosslinked polymer chains. Crosslinks are covalent bonds or other strong interactions such as entanglements, supramolecular interactions, or physical interactions such as polymer chain interactions with crystalline or glassy phases.

“Non-linear force,” as used herein, means a type of force in which the relationship between the force and its effect on a system is not proportional or does not follow a simple, linear equation.

“Non-woven fabric,” as used herein, refers to an assembly of mono-component and/or bi-component fibers (e.g., core/sheath, islands in the sea, side-by side, segmented pie etc.) held together in a random web, such as by mechanical interlocking, or by fusing at least a portion of the fibers.

“On-demand,” as used herein, refers to debonding or detachment that is quick, easy, and damage-free. On-demand delamination or release from the surface to which the medical adhesive composition or article is adhered occurs via reversible or irreversible adhesive behavior triggered by exposure to a stimulus such as temperature changes, physico-chemical changes, light, ultrasound, ionic strength change, pH change, magnetic, or mechanical actions or forces as well as other stimuli.

“Organ,” as used herein, refers to an organ in a physiological system.

“Polymer,” as used herein, refers substances composed of macromolecules, very large molecules with molecular weights ranging from a few thousand to as high as millions of grams/mole made up of simpler, repeating units, derived from lower molecular weight monomers. Polymer, as used herein, refers to both homopolymers and copolymers.

Homopolymers are made from (i.e., they comprise) one type of monomer. Copolymers are made from (i.e., they comprise) two or more different kinds of monomers (e.g., a styrene-butadiene copolymer). Copolymers includes, for example, bio-polymers, terpolymers and quaterpolymers. Copolymers may be block copolymers, graft copolymers, random copolymers, blends, mixtures, and/or adducts of any of the foregoing and other polymers. The adhesive described herein may comprise one or more polymers, including but not limited to stimuli-responsive polymers.

“Plasticizer,” as used herein, refers to an additive that, when added to a polymer, polymer blend, copolymer, copolymer blend, polymer network or copolymer network, results in thermomechanical behavior consistent with that understood to be associated with plasticization, namely, decrease in glass transition temperature, lowering of crystalline melting temperature, triggering of stress relaxation or resulting in increased or decreased adhesion strength. Plasticizers may be added in approximately about 1 wt %, about 2 wt %, about 3 wt % etc., increasing up to about 30 wt % or about 50 wt % or more to polymer, copolymer or network mixtures or blends. Examples of plasticizers for various polymer systems are known an include water, common solvents, small molecules such as phthalates, glycerol or fatty acid compounds, triacetin, poly(ethylene glycol) compounds that are liquid at room temperature with molecular weights ranging from 1 to 30 or more repeat units, vegetable oil, detergents and other common plasticizing agents. Low molecular weight oligomers can also plasticize high molecular weight or crosslinked polymers of the same or similar chemical composition.

“Pressure-sensitive adhesive,” as used herein, refers to an adhesive that gains adhesive performance or tack through polymer flow onto the surface onto which the adhesive is pressed to adhere using force. In certain embodiments herein, the adhesive does not comprise a pressure-sensitive adhesive. The term “conventional pressure sensitive adhesive”, as used in the art, refers to any pressure any pressure sensitive adhesive known in the art, and in some embodiments, refers to any commercially available pressure sensitive adhesive referenced herein or to any pressure sensitive adhesive used in the Examples section herein.

“Reduce” or “reduction,” as used herein, refers to a decrease in a particular property. The reduction may be, for example, at least about 10%, at least about 20%, at least about 30%, at least about 40%, at least about 50%, at least about 60%, at least about 70%, at least about 80%, at least about 90%, or at least about 99% or more. The decrease can be measured by any suitable means.

“Repositionable,” as used herein, refers to the ability to remove a medical adhesive composition (e.g., film or membrane), either partially or completely from a first bodily surface location (e.g., a wound site) and place it in second, different bodily surface location, position, or orientation causing substantially no damage to the medical adhesive composition or first bodily surface location.

“Room temperature” (used interchangeably with “ambient temperature”), as used herein, refers to a temperature from 20-25 °C.

“Scar,” as used herein, refers to a scar is a fibrous tissue that replaces normal tissues destroyed by injury or disease.

“Sealant,” as used herein, refers to a barrier layer to prevent leakage of fluid or gas. In some embodiments, the medical adhesive compositions described herein are sealants. In some embodiments, the industrial or consumer adhesive compositions disclosed herein are useful as sealants.

“Selectively permeable,” as used herein, refers to differential permeability. For example, a membrane contains channels or passages that allow specific molecules to pass through, either passively or actively, while not permitting passage of other molecules. Active transport through the membrane requires an energy input, which may be from a mechanical, acoustic, chemical, electrical, magnetic, pH change, ionic strength, thermal or optical source.

“Self-healing,” as used herein, refers to a class of smart materials that possess the ability to autonomously repair themselves when subjected to mechanical damage or microcracks. This self-repair process can occur without the need for external stimuli or intervention, thereby enhancing the material's durability, reliability, and longevity. Self-healing can occur by flow of viscous or viscoelastic constituents.

“Stimuli-responsive,” as used herein, refers to a change in physical, environmental, physico-chemical, thermomechanical, mechanical, thermal, energetic or other properties of a composition or material arising from exposure to a stimulus including, for example, a change in temperature, pH, ionic strength, environmental conditions including moisture, water immersion, exposure or humidity, solvent exposure, exposure to electromagnetic radiation including gamma rays, x-rays, ultraviolet rays, visible light, infrared waves, radio waves, ultrasonic waves, high humidity, magnetism, electricity, as well as mechanical actions or forces, e.g., shear rate or peeling. In one embodiment, stimuli-responsive does not encompass application of liquid, such as baby oil, to the material to effect the change in properties of the material. In one embodiment, stimuli-responsive refers to a change in physical, environmental, physico-chemical, thermomechanical, mechanical, thermal, energetic or other properties of a composition or material previously applied to a substrate (e.g., skin) arising from exposure to a stimulus. A stimuli-responsive adhesive can be contrasted with a mechanically passive adhesive.

“Sol Fraction” refers to the mass of a polymer lost after washing with a substantial suitable solvent divided by initial mass. Means for determining the sol fraction are known in the art.

“Mechanically passive adhesive” refers to an adhesive that is that are designed to maintain their structural and mechanical properties after placement. Many pressure-sensitive adhesives are mechanically passive adhesives. In certain embodiments, the adhesive layer does not comprise a mechanically passive adhesive or pressure sensitive adhesive.

“Shear rate,” as used herein, refers to the rate at which a shearing deformation occurs. A shearing deformation is one whereby parallel lamellae (layers) of a fluid, gel or solid material moves past one another in a sliding manner. These layers may be discrete (possessing finite measurable thickness) or continuous (infinitesimally thin or not individually discernable). A shear rate may be a constant function in time, a monotonic function in time, a non-periodic non-monotonic or a periodic function in time. Periodic is understood to mean a function that is truly periodic in time, or is approximately periodic in time (e.g., a sine function).

“Shear responsive,” as used herein, refers to behavioral changes of a material (e.g., gel, solid, or liquid) under the application of different shear displacements or shear rates.

“Semi-interpenetrating network” (“Semi-IPN”), as used herein, refers to a crosslinked or branched polymer network and an entangled linear or branched additional polymer or series of polymers.

“Sensor,” as used herein, refers to a device which detects or measures a physical property and records, indicates, or otherwise responds to it. In one embodiment, the article disclosed herein comprises a medical sensor such as a disposable electronic medical sensor, e.g., for use in vital sign monitoring.

“Soft tissue,” as used herein, refers to connective tissue, fatty tissue, fibrous soft tissue or organ tissue. Representative, non-limiting examples of soft tissue include skin, tendons, muscles, ligaments, vessels and the like.

“Skin barrier product,” as used herein, refers to a product used before application of an adhesive to reduce damage to the skin (e.g., silicone barrier films). In one embodiment, the methods of use disclosed herein do not utilize a skin barrier product.

“Subject,” as used herein, refers to the person to which the medical adhesive composition or article is, or is intended to be, applied.

“Substantially no,” as used herein, refers to less than 10% and more particularly, less than 9%, less than 8%, less than 7%, less than 6%, less than 5%, less than 4%, less than 3%, less than 2%, less than 1% or zero, more particularly, less than about 0.9%, less than about 0.8%, less than about 0.7%, less than about 0.6%, less than about 0.5%, less than about 0.4%, less than about 0.3%, less than about 0.2%, less than about 0.1%.

“Tg”, as used herein, refers to the glass transition temperature of a polymer. At this temperature, polymers undergo a transition from glassy to rubbery state. Tg is an important feature of polymer behavior. It marks a region of dramatic changes in the physical and mechanical properties. Below a polymer's Tg, due to lack of mobility, the polymer is hard and brittle. Above a polymer's Tg, due to increased mobility, the polymer is soft and flexible.

“Target site,” “surface,” and “bodily surface” are used herein interchangeably to refer to any surface comprising cells or biological molecules. Non limiting examples of surfaces include a tissue surface, graft surface, or organ surface.

“Treat”, “treatment,” and “treating,” as used herein, refer to (i) a reduction in severity or duration of a disease or disorder, (ii) the amelioration of one or more symptoms associated with aa disease or disorder without necessarily curing the disease or disorder, or (3) the prevention of the disease or disorder.

The term “tube,” as used herein, refers to a structure with one or more lumens, such as a tube, pipe, hose, conduit, intended to convey fluid or the like between two ends. Representative, non-limiting tubes include oxygen tubes, feeding tubes, endotracheal tubes, and nasogastric tubes.

“Wound,” as used herein, refers to an injury to any tissue in the which results in a break and subsequent damage to organ structures, cells, blood vessels, extracellular matrices (ECM), membranes or the like. The injury may be caused, for example, by surgical procedures (e.g., genera surgery, minimally invasive surgery, laparoscopic surgery, biopsy), mechanical damage, burns, ulcers pressure sores, infection, autoimmune or allergic reactions. A wound may range from superficial (e.g., affecting merely the epidermis) to more traumatic (e.g., lesions which affect layers of skin or tissue at depths which are beneath the epidermis). Wounds may be of any length or shape, e.g., in some embodiments, wounds are straight, jagged or curved, full thickness or partial thickness. Wounds may be acute or chronic.

### 4.2. Adhesive

In one aspect, the present disclosure provides an adhesive composition comprising a stimuli-responsive polymer, and optionally one or more other polymers, cross linkers and/or additives, wherein the composition delaminates from a surface (e.g., bodily) surface to which it is adhered following application of a stimulus to the adhesive (e.g., medical adhesive) composition. In one embodiment, the adhesive is suitable for use in a medical context, i.e., a medical adhesive composition. In other embodiments, the adhesive is suitable for use in an industrial, consumer or other context, i.e., a non-medical context.

The medical adhesive compositions described herein have a variety of uses. For example, the medical adhesive composition may be useful as hemostatic material, an air or fluid leak preventive material, an adhesion preventive material, a suture reinforcement, an adhesive sheet, and the like. In some embodiments, the medical adhesive composition is a dental adhesive. In some embodiments, the medical adhesive composition is useful for adhering a medical device to an internal or external surface. For an internal surface, the medical device may be an implantable medical device. For an external surface, the medical device may be a sensor or monitoring device.

In some embodiments, the medical adhesive compositions described herein are useful as bandages, dressings, sealants, glues, coatings, coverings, sensors, drug delivery vehicles or the like. In one embodiment, the medical adhesive composition is not a drug delivery vehicle.

In some embodiments, the medical adhesive composition is useful as a closure device for soft tissue, i.e., reconnecting soft tissue, for example following injury or trauma.

In some embodiments, the medical adhesive composition is useful as a wound dressing or for wound repair. The wound may be any type of wound, including a primary open wound. Wounds include cuts, grazes, abrasions, tears, burns, scalds, ulcers, blemishes, blisters and grazes, whether dermal, epidermal or a combination of both. In some embodiments, the wound is acute or chronic. In some embodiments, the wound is infected.

In some embodiments, the medical adhesive composition is useful as a surgical sealant. As a surgical sealant, the medical adhesive composition may be used to prevent fluid or gas ingress or egress (e.g., bleeding, loss of cerebral spinal fluid) following surgical repair or reconstruction. As a surgical sealant, the medical adhesive composition may be used independently or, or supportive to, other surgical closure or securement devices (e.g., sutures, stapes or tapes). In a particular embodiment, the surgical repair is non-invasive. According to this embodiment, the medical adhesive composition may be provided as a solid or liquid.

The target surface or bodily surface for application of the medical adhesive composition disclosed herein may be an internal or external surface. Examples of external surfaces include the skin or teeth. Examples of internal surfaces include the mucosa, e.g., the oral mucosa or esophageal mucosa and organs.

In some embodiments, the target surface or bodily surface for application of the medical adhesive composition is the skin, e.g., the skin of the face, chest or extremities. In some embodiments, the target surface for application of the medical adhesive composition is an internal structure or organ.

In some embodiments, the medical adhesive compositions are not used in diaphragms or condoms.

In some embodiments, the medical adhesive composition is not in contact with or provided with any final barrier and instead is provided in solid, liquid or other forms to facilitate adhesion between skin, mucosal tissue or other biomass and an additional barrier that includes polyurethane, poly(vinyl chloride), polymeric, latex or other barriers. In a particular embodiment, the medical adhesive composition is applied as a solid film that is adhesive on both sides, after which an additional barrier is applied.

In some embodiments, the medical adhesive composition is in the form of a membrane or film. In some embodiments, the membrane or film exhibits elastomeric or flexible thermomechanical behavior. In some embodiments, the membrane or film has a thickness from 25 μm to 2,000 μm, such as, for example, from 25 μm to 1,500 μm, from 25 μm to 1,000 μm, from 25 μm to 500 μm, from 25 μm to 250 μm, or from 25 μm to 100 μm.

The size of the membrane or film can be tailored for specific intended uses, or it can be provided in a sheet or roll form. In some embodiments, the adhesive composition is provided as a tape.

In some embodiments, the membrane or film ranges in size from about 0.0001 inch to about 2 or 3 inches or more, although preferred widths in embodiments may be from about 4 to about 1 or 14 inches and can range in length from about 4 inch to about 4 or 5 inches or more, although preferred lengths in embodiments may be from about 1 to about 2 or 3 inches. In another embodiment, the membrane or film ranges in size from about 5 to about 8 inches or more.

In some embodiments, the membrane has a planar or curved geometry selected from a square, circle, oval, hemisphere, rectangle, polygon, or curvilinear polygon.

In some embodiments, the membrane has a circle geometry. In some embodiments, the radius of the circle is at least about 0.5 cm, e.g., about 1.0 cm, about 2.0 cm, about 3.0 cm, or about 5.0 cm. In some embodiments, the radius of the circle is about 0.5 cm, about 1.0 cm, about 2.0 cm, about 3.0 cm, about 5.0 cm or about 10.0 cm or greater.

In some embodiments, the membrane has a rectangle geometry. In some embodiments, the rectangle has a length from 0.5 cm to 5 cm and a width from 0.5 cm to 5 cm. In some embodiments, the rectangle has a length from 0.5 cm to 4 cm, from 0.5 cm to 3 cm, from 0.5 cm to 2 cm, from 0.5 cm to 1 cm, from 1 cm to 5 cm, from 1 cm to 4 cm, from 1 cm to 3 cm, from 1 cm to 2 cm, from 2 cm to 5 cm, from 2 cm to 4 cm, from 2 cm to 3 cm, from 3 cm to 5 cm, or from 4 cm to 5. In some embodiments, the rectangle has a width from 0.5 cm to 4 cm, from 0.5 cm to 3 cm, from 0.5 cm to 2 cm, from 0.5 cm to 1 cm, from 1 cm to 5 cm, from 1 cm to 4 cm, from 1 cm to 3 cm, from 1 cm to 2 cm, from 2 cm to 5 cm, from 2 cm to 4 cm, from 2 cm to 3 cm, from 3 cm to 5 cm, or from 4 cm to 5.

In some embodiments, the membrane has an oval geometry. In some embodiments, the oval has a primary radius of about 0.5 cm, about 1.0 cm, about 2.0 cm, about 3.0 cm, about 5.0 cm or about 10.0 cm and a separate secondary radius of about 0.5 cm, about 1.0 cm, about 2.0 cm, about 3.0 cm, about 5.0 cm or about 10.0 cm or greater.

In some embodiments, the membrane has primary dimensions of about 0.5×0.5 cm, about 1.0×1.0 cm, about 1.5×1.5 cm, about 2.5×2.5 cm, about 3.0×3.0 cm, or about 5.0×5.0 cm or any combination thereof in the case of a rectangular membrane.

In some embodiments, the membrane has one or more patterned configurations. Patterning may facilitate adhesive crack propagation or shear-responsive delamination. For example, adhesive patterning may enable sufficient adhesion of the medical adhesive composition to the bodily surface while also enabling peeling with minimal or no pain. Such patterning includes, but is not limited to, a continuous ring or repeating dot ring layer at the base of the membrane ranging from 0.1 to 3000 micrometers in length, more particularly 1 to 2000 micrometers in length, more particularly 20 to 2000 micrometers in length, and ranging from 0.1 to 3000 micrometers thick, more particularly 1 to 2000 micrometers thick, more particularly 5 to 1000 micrometers thick, more particularly 10 to 600 micrometers thick.

Exemplary pattern configurations include rings, stripes, dots, and combinations thereof. Patterning may be uniform, non-uniform or random in shape or size or location, or any combination thereof. In some embodiments, the one or more patterned configuration may cover about 1%, about 5%, about 10%, about 25%, about 33%, about 50%, about 66%, about 75% or about 100% of the membrane.

In some embodiments, the patterned configuration comprises one or more rings or ring-like structures. The thickness of the rings can be, for example, about 0.1 mm, about 0.25 mm, about 0.5 mm, or about 1.0 mm or more. In some embodiments, the ring pattern covers at least about 1%, about 5%, about 10%, about 25%, about 33%, about 50%, about 66%, about 75%, or about 100% of the membrane.

In some embodiments, the patterned configuration comprises dots (e.g., square, rectangular or circular shaped dots). In some embodiments, the dot pattern covers at least about 1%, about 5%, about 10%, about 25%, about 33%, about 50%, about 66%, about 75%, or about 100% of the membrane.

In some embodiments, the pattern configuration comprises stripes. In some embodiments, the strips can be of varying thickness, e.g., about 0.1 mm, about 0.25 mm, about 0.5 mm, or about 1.0 mm or more. In some embodiments, the stripes cover about 1%, about 5%, about 10%, about 25%, about 33%, about 50%, about 66%, about 75% or about 100% of the membrane.

In some embodiments, the membrane has a thickness in the nanometer scale or micron scale. In some embodiments, the membrane has a thickness from 0.1 microns to 3,000 microns, such as, for example, from 0.1 microns to 2,000 microns, from 0.1 microns to 1,000 microns, from 0.1 microns to 750 microns, from 0.1 microns to 500 microns, from 0.1 microns to 250 microns, from 1 microns to 2,000 microns, from 1 microns to 1,000 microns, from 1 microns to 750 microns, from 1 microns to 500 microns, from 1 microns to 250 microns, from 25 microns to 2,000 microns, from 25 microns to 1,000 microns, from 25 microns to 750 microns, from 25 microns to 500 microns, from 25 microns to 250 microns, from 100 microns to 2,000 microns, from 100 microns to 1,000 microns, from 100 microns to 750 microns, from 100 microns to 500 microns, or from 100 microns to 250 microns,

In some embodiments, the membrane has a thickness from about 0.01 microns to about 0.1 microns, from about 1.0 microns to about 5.0 microns, from about 10.0 microns to about 20.0 microns, from about 30.0 microns to about 50.0 microns, from about 100.0 microns to about 200 microns, from about 300 microns to about 400 microns, from about 600 microns to about 750 microns, or from about 1000 microns to about 2000.

In some embodiments, the membrane is from 25 to 750 microns thick, such as, for example, from about 200 to about 500 microns, or about 400 microns thick.

In some embodiment, the membrane has a thickness of about 25 microns, about 50 microns, about 100 microns, about 150 microns, about 200 microns, about 250 microns, about 300 microns, about 350 microns, about 400 microns, about 450 microns, about 500 microns, about 550 microns, about 600 microns, about 650 microns, about 700 microns, or about 750 microns.

In some embodiments, the membrane has a thickness from about 100 microns to about 600 microns, such as, for example, from about 200 microns to about 500 microns, or from about 300 to about 500 microns.

In some embodiments, the medical adhesive composition is transparent. In some embodiments, the membrane is transparent.

In some embodiments, the medical adhesive composition can be repositioned.

In some embodiments, the adhesive (e.g., medical adhesive) composition comprises multiple adhesives, blended or applied separately to a substrate. For example, the adhesive composition may include a hydrophobic, water-insoluble layer at its outer edges along the circumference of the substrate approximately 0.1 to 2 cm in length, more particularly 0.2 to 1 cm in length, more particularly 0.25 to 1 cm in length, and a hydrophilic, water-soluble layer above the hydrophobic layer. In some embodiments, the hydrophobic outer layer may be stimuli-responsive.

In some embodiments, the adhesive (e.g., medical adhesive) composition comprises (1) a primary side chain optionally crystallizable side chain adhesive polymer, (2) optional additives, (3) optionally, one or more additional polymers, and in some embodiments, additional amorphous polymer blended phases or heterophases, and (4) optional crosslinkers, which optionally are incorporated into polymer network uniformly and optionally are aggregated and serve as high stress concentration network sites to facilitate adhesive failure or desirable tack profile when desirable. In some embodiments, the adhesive composition comprises a blend or copolymer of amorphous polymers that may crystallize.

In some embodiments, the adhesive (e.g., medical adhesive) composition described herein comprises a stimuli-responsive polymer formed from one or more monomers and optionally one or more polyfunctional crosslinkers. In some embodiments, the adhesive (e.g., medical adhesive) composition consists of the stimuli-responsive polymer. In some embodiments, the adhesive (e.g., medical adhesive) composition consists essentially of the stimuli-responsive polymer. “Consists essentially of,” as used herein with respect to the adhesive composition, refers to additional components that do not materially affect the basic characteristics of the composition, e.g., additives that do not have adhesive properties themselves.

In some embodiments, the adhesive (e.g., medical adhesive) composition does not include additional components, e.g., additives, adhesive polymers, and/or other polymers.

In some embodiments, the medical adhesive composition does not comprise cyanoacrylates.

In some embodiments, the medical adhesive composition does not comprise fibrin.

In some embodiments, the medical adhesive composition comprises one or more therapeutic agents, including but not limited to, anti-infectives (e.g., antibiotics, antivirals, antifungals), antioxidants (e.g., vitamins), anti-inflammatory agents, hemostatic agents (e.g., prothrombin, thrombin, silicate nanoparticles), pain-reducing agents (e.g., analgesics and anesthetics, such as local anesthetic agents), growth factors, blotting clotting of vasoconstrictive agents, and scar reducing agents.

In some embodiments, the medical adhesive composition comprises an antibiotic selected from bacitracin, clindamycin, erythromycin, mupirocin, neomycin, penicillin, polymycin B, tetracycline, colloidal silver, silver salts, silver sulfadiazine, polyhexanide, chlorhexidine, povidone iodine, triclosan, sucralfate, quaternary ammonium salts, and combinations thereof.

In some embodiments, the pain-reducing agent may be a topical anesthetic such as lidocaine, tetracaine, prilocaine or the like.

In some embodiments, the adhesive (e.g., medical adhesive) composition comprises two or three or more adhesive regions which may exhibit different mechanical, chemical and biological properties. These may include different solubility parameters in various solvents including water or bodily fluids. In some embodiments, the at least one therapeutic agent can be incorporated into a separate layer of the medical adhesive composition.

The adhesive (e.g., medical adhesive) composition may comprise a single layer or multiple layers. In some embodiments, adhesive (e.g., medical adhesive) composition comprises a single layer. In some embodiments, the adhesive (e.g., medical adhesive) composition comprises multiple layers. The layers may be the same or different.

The industrial adhesive compositions described herein have a variety of uses, including, but not limited to, use as a sealant. In one embodiment, the industrial adhesive composition is useful as a flow system adhesive or sealant. In some embodiments, the adhesive compositions disclosed herein are also useful in consumer products. In an additional non-limiting embodiment, the industrial sealant is not manufactured using 3D printing techniques.

### 4.2.1.1 Adhesive Properties

In some embodiments, the adhesive composition is applied to a surface, e.g., a glass, ceramic, composite, plastic/polymeric or metal surface.

In some embodiments, when the medical adhesive composition is applied to a target site or bodily surface, the medical adhesive composition adheres to the target site or bodily surface. Exemplary bodily surfaces include, but are not limited to, tissues and organs. In some embodiments, the bodily surface is a skin surface, e.g., the skin of the head, neck, chest, upper limb, lower limb, hand, foot, abdomen, torso, back, buttocks, and genitals.

Application of a stimulus to the adhesive (e.g., medical adhesive) composition causes the stimuli-responsive polymer to become less adhesive or delaminate from the surface (e.g., bodily surface) to which it was previously attached, thereby enabling selective delamination (i.e., on-demand delamination). Reversible or irreversible adhesive behavior can be triggered by exposure to temperature changes (e.g., a decrease in temperature), physico-chemical changes (e.g., dissolution), light, ultrasound, ionic strength change, pH change, magnetic, electrical, or mechanical actions or forces, as well as other stimuli or any combination thereof.

Properties for the stimuli-responsive polymer recited below may also apply to the adhesive (e.g., medical adhesive) composition, which can optionally contain additional components, e.g., additives and/or polymers (e.g., adhesive polymers or non-adhesive polymers).

In some embodiments, the stimuli-responsive polymer becomes less adhesive or delaminates from the surface (e.g., bodily surface) in 0.1 s to 60 s in response to the stimulus, such as, for example, from 1 s to 60 s, from 1 s to 30 s, from 1 s to 15 s, from 1 s to 10 s, from 1 s to 5 s, from 2 s to 30 s, or from 1 s to 15 s.

In some embodiments, the adhesive (e.g., medical adhesive) composition becomes less adhesive or delaminates from the surface (e.g., bodily surface) in 0.1 s to 60 s in response to the stimulus, such as, for example, from 1 s to 60 s, from 1 s to 30 s, from 1 s to 15 s, from 1 s to 10 s, from 1 s to 5 s, from 2 s to 30 s, or from 1 s to 15 s.

In some embodiments, a stimuli-responsive polymer delaminates from the surface (e.g., bodily surface) faster than a non-stimuli-responsive polymer, e.g., a conventional pressure-sensitive adhesive polymer. In some embodiments, a stimuli-responsive polymer delaminates at least 2× faster than a non-stimuli-responsive polymer, e.g., a conventional pressure-sensitive adhesive polymer, such as, for example, at least 3× faster, at least 4× faster, at least 5× faster, at least 6× faster, at least 7× faster, at least 8× faster, at least 9× faster, or at least 1OX faster.

In some embodiments, the adhesive (e.g., medical adhesive) composition comprising a stimuli-responsive polymer delaminates from the surface (e.g., bodily surface) faster than an adhesive (e.g., medical adhesive) composition that does not comprise a stimuli-responsive polymer, e.g., a conventional pressure-sensitive adhesive polymer. In some embodiments, an adhesive (e.g., medical adhesive) composition comprising a stimuli-responsive polymer delaminates at least 2× faster than an adhesive (e.g., medical adhesive) composition that does not comprise a stimuli-responsive polymer, e.g., a conventional pressure-sensitive adhesive polymer, such as, for example, at least 3× faster, at least 4× faster, at least 5× faster, at least 6× faster, at least 7× faster, at least 8× faster, at least 9× faster, or at least 1OX faster.

In some embodiments, the stimulus is a mechanical action, e.g., shear rate. In some embodiments, the shear rate is induced by peeling or pulling on the adhesive (e.g., medical adhesive) composition at different rates or frequencies.

In some embodiments, the stimuli-responsive polymer is shear-rate responsive. In some embodiments, the adhesive (e.g., medical adhesive) composition is shear-rate responsive. In such embodiments, the stimuli-responsive polymer remains adhered upon application of higher shear rates and becomes less adhesive at lower shear rates. Exemplary higher shear rates include hard peeling or hard pulling. Exemplary lower shear rates include light peeling or light pulling. Higher and lower are taken with respect to a threshold about which a change in behavior is observed. In this manner, the stimuli-responsive polymer and the adhesive (e.g., medical adhesive) composition can be easily removed from the surface (e.g., bodily surface) following application of a stimulus to the adhesive (e.g., medical adhesive) composition.

In some embodiments, light peeling corresponds to a peel rate of 500 mm/min or less, such as, for example, 400 mm/min or less, 300 mm/min or less, 200 mm/min or less, 100 mm/min or less, or 50 mm/min or less. In some embodiments, light peeling corresponds to a peel rate of 50 mm/min to 500 mm/min, such as, for example, from 50 mm/min to 400 mm/min, from 50 mm/min to 300 mm/min, from 50 mm/min to 200 mm/min, from 50 mm/min to 100 mm/min, from 100 mm/min to 500 mm/min, from 100 mm/min to 400 mm/min, from 100 mm/min to 300 mm/min. from 100 mm/min to 200 mm/min, from 200 mm/min to 500 mm/min, from 200 mm/min to 400 mm/min, from 200 mm/min to 300 mm/min, from 300 mm/min to 500 mm/min, from 300 mm/min to 400 mm/min, or from 400 mm/min to 500 mm/min.

In some embodiments, light peeling corresponds to a peel rate of 25 mm/s or less, such as, for example, 10 mm/s or less, 5 mm/s or less, 1 mm/s or less, 0.5 mm/s or less, 0.3 mm/s or less, or 0.1 mm/s or less. In some embodiments, light peeling corresponds to a peel rate from 0.01 mm/s to 25 mm/s, such as, for example, from 0.01 mm/s to 10 mm/s, from 0.01 mm/s to 5 mm/s, from 0.01 mm/s to 1 mm/s, from 0.01 mms/to 0.5 mm/s, from 0.01 mm/s to 0.3 mm/s, from 0.01 mm/s to 0.1 mm/sec, from 0.1 mm/s to 25 mm/s, from 0.1 mm/s to 10 mm/s, from 0.1 mm/s to 5 mm/s, from 0.1 mm/s to 1 mm/s, from 0.1 mms/to 0.5 mm/s, from 0.1 mm/s to 0.3 mm/s, from 1 mm/s to 25 mm/s, from 1 mm/s to 10 mm/s, or from 1 mm/s to 5 mm/s.

In some embodiments, the stimulus is a mechanical force. In some embodiments, the stimuli-responsive polymer is force responsive. In some embodiments, the adhesive (e.g., medical adhesive) composition is force responsive. In such embodiments, the stimuli-responsive polymer remains adhered upon application of higher forces and becomes less adhesive upon application of lower forces. Higher and lower are taken with respect to a threshold about which a change in behavior is observed.

In some embodiments, the applied force that results in delamination of the adhesive (e.g., medical adhesive) composition is, for example, from 0.01 to 0.1 N, from 0.1 to 1 N, from 1 to 10 N, from 10 to 100 N, or from 100 and 1000 N. In some embodiments, the applied force that results in delamination is, for example, from 1 to 10 Pa, from 10 to 100 Pa, from 0.1 to 1 kPa, from 1 to 10 kPa, from 10 to 100 kPa, or from 0.1 to 1 MPa.

In some embodiments, the applied force that results in delamination of the stimuli-responsive polymer is, for example, from 0.01 to 0.1 N, from 0.1 to 1 N, from 1 to 10 N, from 10 to 100 N, or from 100 and 1000 N. In some embodiments, the applied force that results in delamination is, for example, from 1 to 10 Pa, from 10 to 100 Pa, from 0.1 to 1 kPa, from 1 to 10 kPa, from 10 to 100 kPa, or from 0.1 to 1 MPa.

In some embodiments, the stimuli-responsive polymer has a lower peel strength at lower peel rates and a higher peel strength at higher peel rates. In some embodiments, the stimuli-responsive polymer has at least a 5% lower peel strength at a lower peel rate compared to the peel strength at a higher peel rate, e.g., at least 10% lower, at least 20% lower, at least 30% lower, at least 40% lower, at least 50% lower, at least 60% lower, at least 70% lower, at least 80% lower, at least 90% lower, or at least 100% lower.

In some embodiments, the stimuli-responsive polymer has a lower peel strength at a peel rate of 100 mm/min than at 200 mm/min. In some embodiments, the stimuli-responsive polymer has a lower peel strength at a peel rate of 100 mm/min than at 300 mm/min. In some embodiments, the stimuli-responsive polymer has a lower peel strength at a peel rate of 100 mm/min than at 400 mm/min. In some embodiments, the stimuli-responsive polymer has a lower peel strength at a peel rate of 100 mm/min than at 500 mm/min.

In some embodiments, the stimuli-responsive polymer has a lower peel strength at a peel rate of 1 mm/s than at 3 mm/s. In some embodiments, the stimuli-responsive polymer has a lower peel strength at a peel rate of 1 mm/s than at 5 mm/s. In some embodiments, the stimuli-responsive polymer has a lower peel strength at a peel rate of 1 mm/s than at 7 mm/s. In some embodiments, the stimuli-responsive polymer has a lower peel strength at a peel rate of 1 mm/s than at 10 mm/s.

In some embodiments, the stimuli-responsive polymer has at least a 5% lower peel strength at a peel rate of 100 mm/min than at 200 mm/min, e.g., at least 10% lower, at least 20% lower, at least 30% lower, at least 40% lower, at least 50% lower, at least 60% lower, at least 70% lower, at least 80% lower, at least 90% lower, or at least 100% lower.

In some embodiments, the stimuli-responsive polymer has at least a 5% lower peel strength at a peel rate of 100 mm/min than at 300 mm/min, e.g., at least 10% lower, at least 20% lower, at least 30% lower, at least 40% lower, at least 50% lower, at least 60% lower, at least 70% lower, at least 80% lower, at least 90% lower, or at least 100% lower.

In some embodiments, the stimuli-responsive polymer has at least a 5% lower peel strength at a peel rate of 100 mm/min than at 400 mm/min, e.g., at least 10% lower, at least 20% lower, at least 30% lower, at least 40% lower, at least 50% lower, at least 60% lower, at least 70% lower, at least 80% lower, at least 90% lower, or at least 100% lower.

In some embodiments, the stimuli-responsive polymer has at least a 5% lower peel strength at a peel rate of 100 mm/min than at 500 mm/min, e.g., at least 10% lower, at least 20% lower, at least 30% lower, at least 40% lower, at least 50% lower, at least 60% lower, at least 70% lower, at least 80% lower, at least 90% lower, or at least 100% lower.

In some embodiments, the stimuli-responsive polymer has a lower peel strength at lower peel rates and a higher peel strength at higher peel rates. In some embodiments, the adhesive (e.g., medical adhesive) composition has at least a 5% lower peel strength at a lower peel rate compared to the peel strength at a higher peel rate, e.g., at least 10% lower, at least 20% lower, at least 30% lower, at least 40% lower, at least 50% lower, at least 60% lower, at least 70% lower, at least 80% lower, at least 90% lower, or at least 100% lower.

In some embodiments, the stimuli-responsive polymer has at least a 5% lower peel strength at a peel rate of 1 mm/s than at 3 mm/s, e.g., at least 10% lower, at least 20% lower, at least 30% lower, at least 40% lower, at least 50% lower, at least 60% lower, at least 70% lower, at least 80% lower, at least 90% lower, or at least 100% lower.

In some embodiments, the stimuli-responsive polymer has at least a 5% lower peel strength at a peel rate of 1 mm/s than at 5 mm/s, e.g., at least 10% lower, at least 20% lower, at least 30% lower, at least 40% lower, at least 50% lower, at least 60% lower, at least 70% lower, at least 80% lower, at least 90% lower, or at least 100% lower.

In some embodiments, the stimuli-responsive polymer has at least a 5% lower peel strength at a peel rate of 1 mm/s than at 7 mm/s, e.g., at least 10% lower, at least 20% lower, at least 30% lower, at least 40% lower, at least 50% lower, at least 60% lower, at least 70% lower, at least 80% lower, at least 90% lower, or at least 100% lower.

In some embodiments, the adhesive (e.g., medical adhesive) composition has a lower peel strength at lower peel rates and a higher peel strength at higher peel rates. In some embodiments, the adhesive (e.g., medical adhesive) composition has at least a 5% lower peel strength at a lower peel rate compared to the peel strength at a higher peel rate, e.g., at least 10% lower, at least 20% lower, at least 30% lower, at least 40% lower, at least 50% lower, at least 60% lower, at least 70% lower, at least 80% lower, at least 90% lower, or at least 100% lower.

In some embodiments, the adhesive (e.g., medical adhesive) composition has a lower peel strength at a peel rate of 100 mm/min than at 200 mm/min. In some embodiments, the adhesive (e.g., medical adhesive) composition has a lower peel strength at a peel rate of 100 mm/min than at 300 mm/min. In some embodiments, the adhesive (e.g., medical adhesive) composition has a lower peel strength at a peel rate of 100 mm/min than at 400 mm/min. In some embodiments, the adhesive (e.g., medical adhesive) composition has a lower peel strength at a peel rate of 100 mm/min than at 500 mm/min.

In some embodiments, the adhesive (e.g., medical adhesive) composition has a lower peel strength at a peel rate of 1 mm/s than at 3 mm/s. In some embodiments, the adhesive (e.g., medical adhesive) composition has a lower peel strength at a peel rate of 1 mm/s than at 5 mm/s. In some embodiments, the adhesive (e.g., medical adhesive) composition has a lower peel strength at a peel rate of 1 mm/s than at 7 mm/s. In some embodiments, the adhesive (e.g., medical adhesive) composition has a lower peel strength at a peel rate of 1 mm/s than at 10 mm/s.

In some embodiments, the adhesive (e.g., medical adhesive) composition has at least a 5% lower peel strength at a peel rate of 100 mm/min than at 200 mm/min, e.g., at least 10% lower, at least 20% lower, at least 30% lower, at least 40% lower, at least 50% lower, at least 60% lower, at least 70% lower, at least 80% lower, at least 90% lower, or at least 100% lower.

In some embodiments, the adhesive (e.g., medical adhesive) composition has at least a 5% lower peel strength at a peel rate of 100 mm/min than at 300 mm/min, e.g., at least 10% lower, at least 20% lower, at least 30% lower, at least 40% lower, at least 50% lower, at least 60% lower, at least 70% lower, at least 80% lower, at least 90% lower, or at least 100% lower.

In some embodiments, the adhesive (e.g., medical adhesive) composition has at least a 5% lower peel strength at a peel rate of 100 mm/min than at 400 mm/min, e.g., at least 10% lower, at least 20% lower, at least 30% lower, at least 40% lower, at least 50% lower, at least 60% lower, at least 70% lower, at least 80% lower, at least 90% lower, or at least 100% lower.

In some embodiments, the adhesive (e.g., medical adhesive) composition has at least a 5% lower peel strength at a peel rate of 100 mm/min than at 500 mm/min, e.g., at least 10% lower, at least 20% lower, at least 30% lower, at least 40% lower, at least 50% lower, at least 60% lower, at least 70% lower, at least 80% lower, at least 90% lower, or at least 100% lower.

In some embodiments, the adhesive (e.g., medical adhesive) composition has at least a 5% lower peel strength at a peel rate of 1 mm/s than at 3 mm/s, e.g., at least 10% lower, at least 20% lower, at least 30% lower, at least 40% lower, at least 50% lower, at least 60% lower, at least 70% lower, at least 80% lower, at least 90% lower, or at least 100% lower.

In some embodiments, the adhesive (e.g., medical adhesive) composition has at least a 5% lower peel strength at a peel rate of 1 mm/s than at 5 min/s, e.g., at least 10% lower, at least 20% lower, at least 30% lower, at least 40% lower, at least 50% lower, at least 60% lower, at least 70% lower, at least 80% lower, at least 90% lower, or at least 100% lower.

In some embodiments, the adhesive (e.g., medical adhesive) composition has at least a 5% lower peel strength at a peel rate of 1 mm/s than at 7 mm/s, e.g., at least 10% lower, at least 20% lower, at least 30% lower, at least 40% lower, at least 50% lower, at least 60% lower, at least 70% lower, at least 80% lower, at least 90% lower, or at least 100% lower.

In some embodiments, the stimuli-responsive polymer has a peel strength from 1 to 400 N/m at a peel rate of 100 mm/sec, such as, for example, from 1 to 300 N/m, from 1 to 200 N/m, from 1 to 100 N/m, or from 1 to 50 N/m. Peel strength can be determined by a 180° peel test using a human skin substrate analog as described hereinbelow.

In some embodiments, the adhesive (e.g., medical adhesive) composition has a peel strength from 1 to 400 N/m at a peel rate of 100 mm/sec, such as, for example, from 1 to 300 N/m, from 1 to 200 N/m, from 1 to 100 N/m, or from 1 to 50 N/m. Peel strength can be determined by a 1800 peel test using a human skin substrate analog as described hereinbelow.

In some embodiments, the stimuli-responsive polymer has a tack strength at 25 °C of at least 1 N. such as, for example, from 1 N to 5 N, from 1 N to 4 N, from 1 N to 3 N, or from 1 N to 2 N. Tack strength can be measured as described hereinbelow, e.g., compressing an aluminum-tipped rheometer containing the polymer to an aluminum base plate for 60 s, withdrawing the rheometer at 100 micrometers per second, and measuring the axial force in N.

In some embodiments, the adhesive (e.g., medical adhesive) composition has a tack strength at 25 °C of at least 1 N, such as, for example, from 1 N to 5 N, from 1 N to 4 N, from 1 N to 3 N, or from 1 N to 2 N.

In some embodiments, the stimuli-responsive polymer has an adhesive strength at 25 °C of a least 20 N*s, such as, for example, at least 30 N*s, at least 40 N*s, at least 50 N*s, at least 60 N*s, at least 70 N*s, at least 80 N*s, at least 90 N*s, at least 100 N*s, at least 150 N*s, at least 200 N*s, at least 300 N*s, at least 400 N*s, or at least 500 N*s. Adhesive strength is calculated by integrating the area under the axial force vs time measurements for a sample.

In some embodiments, the adhesive (e.g., medical adhesive) composition has an adhesive strength at 25 °C of a least 20 N*s, such as, for example, at least 30 N*s, at least 40 N*s, at least 50 N*s, at least 60 N*s, at least 70 N*s, at least 80 N*s, at least 90 N*s, at least 100 N*s, at least 150 N*s, at least 200 N*s, at least 300 N*s, at least 400 N*s, or at least 500 N*s.

In some embodiments, the stimuli-responsive polymer has a storage modulus of from 0.01 MPa to 1 MPa, such as, for example, from 0.1 MPa to 1 MPa. from 0.1 MPa to 0.8 MPa or from 0.1 MPa to 0.5 MPa.

In some embodiments, the adhesive (e.g., medical adhesive) composition has a storage modulus of from 0.01 MPa to 1 MPa, such as, for example, from 0.1 MPa to 1 MPa, from 0.1 MPa to 0.8 MPa or from 0.1 MPa to 0.5 MPa.

In some embodiments, the stimuli-responsive polymer has a loss modulus of from 0.1 MPa to 1 MPa, such as, for example, from 0.1 MPa to 0.8 MPa or from 0.1 MPa to 0.5 MPa.

In some embodiments, the adhesive (e.g., medical adhesive) composition has a loss modulus of from 0.1 MPa to 1 MPa, such as, for example, from 0.1 MPa to 0.8 MPa or from 0.1 MPa to 0.5 MPa.

In some embodiments, the stimuli-responsive polymer has a tan(δ) (ratio of storage modulus (G″) to loss modulus (G′)) at 25 °C and 1 Hz (2*pi radian/s) of at least 0.1, such as, for example, at least 0.2, at least 0.3, at least 0.4 at least 0.5, at least 0.6, at least 0.7, at least 0.8, at least 0.9, at least 1.0, at least 1.1, at least 1.2, at least 1.3, at least 1.4, at least 1.5, at least 1.6, at least 1.7, at least 1.8, at least 1.9, or at least 2.0. In some embodiments, the stimuli-responsive polymer has a tan(δ) from 0.1 to 5, such as, for example, from 0.1 to 4, 0.1 to 3, 0.1 to 2, 0.1 to 1, 0.5 to 5, 0.5 to 4, 0.5 to 3, 0.5 to 3, 0.5 to 2, 0.5 to 1, 1 to 5, 1 to 4, 1 to 3, 1 to 2, 2 to 5, 2 to 4, 2 to 3, 3 to 5, 3 to 4, or 4 to 5. In certain embodiments, the stimuli-responsive polymer has a tan(δ) of 0.2 to 2, e.g., 0.1 to 1.5, 0.1 to 1,0.1 to 0.5, 0.3 to 1, or 0.5 to 1.

In some embodiments, the adhesive (e.g., medical adhesive) composition has a tan(δ) (ratio of storage modulus (G″) to loss modulus (G′)) at 25 °C and 1 Hz (2*radian/s) of at least 0.1, such as, for example, at least 0.2, at least 0.3, at least 0.4 at least 0.5, at least 0.6, at least 0.7, at least 0.8, at least 0.9, at least 1.0, at least 1.1. at least 1.2, at least 1.3, at least 1.4, at least 1.5, at least 1.6, at least 1.7, at least 1.8, at least 1.9, or at least 2.0. In some embodiments, the adhesive (e.g., medical adhesive) composition has a tan(s) from 0.1 to 5, such as, for example, from 0.1 to 4, 0.1 to 3, 0.1 to 2, 0.1 to 1, 0.5 to 5, 0.5 to 4, 0.5 to 3, 0.5 to 3, 0.5 to 2, 0.5 to 1, 1 to 5, 1 to 4, 1 to 3, 1 to 2, 2 to 5, 2 to 4, 2 to 3, 3 to 5, 3 to 4, or 4 to 5. In certain embodiments, the adhesive (e.g., medical adhesive) composition has a tan(δ) of 0.2 to 2, e.g., 0.1 to 1.5, 0.1 to 1, 0.1 to 0.5, 0.3 to 1, or 0.5 to 1.

In some embodiments, the adhesive (e.g., medical adhesive) composition has less tack when wet compared to when dry. In some embodiments, the stimuli-responsive polymer has less tack when wet compared to when dry.

In some embodiments, the stimuli-responsive polymer is a crosslinked polymer is a low-density crosslinked polymer or a polymer with limited, heterogeneous crosslinking.

In some embodiments, the stimuli-responsive polymer has gel fraction consistent with that of a lightly crosslinked polymer network when analyzed by sol-gel analysis. In some embodiments, the stimuli-responsive polymer has a gel fraction from 0.0001 to 0.99, such as, for example, from 0.001 to 0.98, from 0.01 to 0.95, from 0.015 to 0.95, from 0.02 to 0.96, from 0.05 to 0.95, from 0.1 to 0.95, from 0.175 to 0.95, from 0.2 to 0.95, from 0.25 to 0.95, from 0.3 to 0.95, from 0.35 to 0.95, from 0.4 to 0.95, from 0.5 to 0.95, from 0.6 to 0.95, from 0.7 to 0.95, from 0.8 to 0.95, from 0.5 to 0.95, from 0.6 to 0.93, from 0.6 to 0.65 to 0.92, from 0.7 to 0.9, or from 0.7 to 0.89.

In some embodiments, the adhesive (e.g., medical adhesive) composition has gel fraction consistent with that of a lightly crosslinked polymer network when analyzed by sol-gel analysis. In some embodiments, the adhesive (e.g., medical adhesive) composition has a gel fraction from 0.0001 to 0.99, such as, for example, from 0.001 to 0.98, from 0.01 to 0.95, from 0.015 to 0.95, from 0.02 to 0.96, from 0.05 to 0.95, from 0.1 to 0.95, from 0.175 to 0.95, from 0.2 to 0.95, from 0.25 to 0.95, from 0.3 to 0.95, from 0.35 to 0.95, from 0.4 to 0.95, from 0.5 to 0.95, from 0.6 to 0.95, from 0.7 to 0.95, from 0.8 to 0.95, from 0.5 to 0.95, from 0.6 to 0.93, from 0.6 to 0.65 to 0.92, from 0.7 to 0.9, or from 0.7 to 0.89.

In some embodiments, the stimulus is a physico-chemical change. An exemplary physico-chemical change is dissolution. In some embodiments, the stimulus is a change in dissolution of the stimuli-responsive polymer when contacted by a solvent. In some embodiments, the medical adhesive composition adheres to the bodily surface in the absence of the solvent and is less adhesive or delaminates from the bodily surface when contacted by the solvent.

In some embodiments, the adhesive (e.g., medical adhesive) composition exhibits chemically-responsive behavior and may be water or solvent soluble in nature and may be removed by pulling back edges and flowing water or solvent or rubbing a substrate impregnated with water or solvent until the adhesive (e.g., medical adhesive) composition is removed by weakening of stimuli-responsive polymer or dissolution by water or solvent. Chemical delamination may also be achieved in this manner by pH-triggerable delamination by flowing or wiping in this manner with a substrate impregnated with a fluid with a pH suitable for, e.g., skin contact, that also achieves delamination of the composition or article from skin. Alternatively, chemical delamination may also be achieved by dissolution of the adhesive (e.g., medical adhesive) composition by flowing or wiping in this manner with a substrate impregnated with a fluid that achieves delamination from skin.

In some embodiments, the stimulus is temperature change. In some embodiments, the adhesive (e.g., medical adhesive) composition adheres to a surface (e.g., bodily surface) at a temperature of 370 C and is less adhesive or delaminates from the surface (e.g., bodily surface) at a temperature of 25 °C or lower. In some embodiments, the stimuli-responsive polymer adheres to a surface (e.g., bodily) surface at a temperature of 370 C and is less adhesive or delaminates from the surface (e.g., bodily) surface at a temperature of 25 °C or lower.

In some embodiments, the adhesive (e.g., medical adhesive composition) exhibits thermally-responsive stimuli-responsive behavior that enables adhesion to a surface (e.g., bodily surface) at body temperature (about 37 °C) and also enables lowered adhesive force or delamination upon cooling below body temperature to about 25 °C, about 20 °C, about 15 °C, about 10 °C, about 5 °C, about 0 °C or lower. Cooling may be achieved, for example, by accelerated heat transfer by rubbing a wet substrate such as a water-impregnated towel or paper towel on the adhesive or at the interface of the adhered substrate while adhered to the bodily surface or by running water from a source such as a shower or a cloth or tissue wipe which contains a reagent which causes cooling (e.g., alcohol evaporation or dissolution of ammonium nitrate) to cool the adhesive through a thermal transition such that delamination is achievable more easily than if the adhesive were to be removed at body temperature.

In some embodiments, following adhesion of the medical adhesive composition to the bodily surface, removal of the composition by light peeling does not cause pain in the subject. In some embodiments, following adhesion of the medical adhesive composition to the skin, removal of the composition by light peeling causes minimal or no pain to the subject. Any suitable method can be utilized to determine the existence or absence of pain. There are several validated instruments for the measurement of pain. The instruments can be unidimensional, measuring only the intensity of pain, such as Wong-Baker Qualitative Pain Assessment (WBQPA), Numeric Pain Rating Scale (NRPS), visual analogue scale (VAS) and verbal rating scale. Multidimensional instruments measure the intensity, character and impact of pain, for example, the McGill Pain Questionnaire (MPQ) and Brief Pain Inventory (BPI).

In some embodiments, pain is measured using the WBQPA on a scale of 0 (no pain) to 10 (maximum pain). In some embodiments, the pain is less than 4 on the WBQPA scale when the medical adhesive composition is removed by light peeling, e.g., less than 3, less than 2, less than 1 or 0. In some embodiments, a WBQPA score of 0 is reported upon removal of the medical adhesive composition by light peeling.

As discussed above, in some embodiments, light peeling corresponds to a user peel rate of the adhesive (e.g., medical adhesive) composition of 25 mm/s or less, such as, for example, 10 mm/s or less, 5 mm/s or less, 1 mm/s or less, 0.5 mm/s or less, 0.3 mm/s or less, or 0.1 mm/s or less. In some embodiments, light peeling corresponds to a peel rate from 0.01 mm/s to 25 mm/s, such as, for example, from 0.01 mm/s to 10 mm/s, from 0.01 mm/s to 5 mm/s, from 0.01 mm/s to 1 mm/s, from 0.01 mms/to 0.5 mm/s, from 0.01 mm/s to 0.3 mm/s, from 0.01 mm/s to 0.1 mm/sec, from 0.1 mm/s to 25 mm/s, from 0.1 mm/s to 10 mm/s, from 0.1 mm/s to 5 mm/s, from 0.1 mm/s to 1 mm/s, from 0.1 mms/to 0.5 mm/s, from 0.1 mm/s to 0.3 mm/s, from 1 mm/s to 25 mm/s, from 1 mm/s to 10 mm/s, or from 1 mm/s to 5 mm/s.

In some embodiments, light peeling corresponds to a user peel rate of the adhesive (e.g., medical adhesive) composition of 500 mm/min or less, such as, for example, 400 mm/min or less, 300 mm/min or less, 200 mm/min or less, 100 mm/min or less, or 50 mm/min or less, or 25 mm/min or less, or 10 mm/min or less, or 5 mm/min or less, or 1 mm/min or less, or 0.5 mm/min or less, or 0.3 mm/min or less, or 0.1 mm/min or less. In some embodiments, light peeling corresponds to a peel rate of 50 mm/min to 500 mm/min, such as, for example, from 50 mm/min to 400 mm/min, from 50 mm/min to 300 mm/min, from 50 mm/min to 200 mm/min, from 50 mm/min to 100 mm/min, from 100 mm/min to 500 mm/min, from 100 mm/min to 400 mm/min, from 100 mm/min to 300 mm/min, from 100 mm/min to 200 mm/min, from 200 mm/min to 500 mm/min, from 200 mm/min to 400 mm/min, from 200 mm/min to 300 mm/min, from 300 mm/min to 500 mm/min, from 300 mm/min to 400 mm/min, or from 400 mm/min to 500 mm/min.

In some embodiments, following adhesion of the adhesive layer to the bodily surface, removal of the medical adhesive composition by light peeling causes minimal or no pain in the subject when removed in less than 15 seconds, e.g., less than 10 seconds, less than 8 seconds or less than 5 seconds.

In some embodiments, the site of adhesion exhibits limited or no irritation upon removal. Various methods are known in research settings for quantitatively assessing skin damage. Certain of these models are based on measurement of baseline skin properties that change when skin is stressed. Measurable parameters such as skin hydration, trans-epidermal water low (TEWL), and irritation, among others, will provide information differentiating damaged skin from healthy skin. See, e.g., Bernatchez, S. et al, ADVANCES IN WOUND CARE, Vol. 2, No. 4 (2022), which is incorporated herein by reference. Reconstructed human epidermis models are also available that demonstrate reasonable similarities to the native human tissue in terms of morphology, lipid composition and biochemical markers. See, e.g., EpiSkin, SkinEthic and EpiDerm. Animal models of skin injury are also known in the art, including pigs and rodents. See, e.g., Summerfield, A. et al., Molecular Immunology, Vol. 66, Issue 1, July 2015, p. 14-21.

### 4.2.1.2 Polymeric Composition

The adhesive disclosed herein comprises a stimuli-responsive polymer, and optionally, one or more additional polymers.

In some embodiments, the stimuli-responsive polymer comprises a single copolymer. In some embodiments, the stimuli-responsive polymer comprises a blend of two or more homopolymers or copolymers, which includes copolymer architecture of block, gradient, and random copolymers of two or more.

In some embodiments, the stimuli-responsive polymer comprises one or more polyacrylate or polymethacrylate polymers.

In some embodiments, the stimuli-responsive polymer comprises one or more polyacrylates or polymethacrylates with 1, 2, 3, 4, 5, 6, 7, 8, 9, 10, 11, 12 or up to 100 carbons in the side chain and 0, 1, 2, 3, 4, 5, 6, 7, 8, 9 10 or up to 100 or more oxygens in the side chain.

Representative, non-limiting polymers include poly(2-ethylhexyl acrylate), poly(butyl acrylate), poly(propyl acrylate), poly(ethyl acrylate), poly(methyl acrylate), poly(octyl acrylate), poly(nonyl acrylate) poly(decyl acrylate), poly(undecyl acrylate), poly(dodecyl acrylate), poly(tridecyl acrylate), poly(C14 acrylate), poly (C15 acrylate), poly(C16 acrylate), poly (C17 acrylate), poly(C18 acrylate), poly(C19 acrylate), poly(C20-C100 or more acrylates, methacrylates and acrylamides thereof), poly(2-hydroxyethyl acrylate), poly(butoxymethyl acrylate), poly(butoxyethyl acrylate), poly(butoxypropyl acrylate), poly(butoxybutyl acrylate), poly(fin(ding)-nemo-acrylate), poly(octadecyl acrylate), poly(octadecyl methacrylate), and poly(acrylic acid).

In some embodiments, the stimuli-responsive polymer comprises polymethacrylate, a polymethacrylate copolymer or blends thereof. The copolymer is derived least one methacrylate monomer and at least one polymerizable comonomer, including any monomer disclosed herein.

In some embodiments, the stimuli-responsive polymer comprises a crosslinked polymethacrylate. The crosslinker can be polyfunctional. In one embodiment, the stimuli-responsive polymer comprises a polymethacrylate crosslinked by an acrylate. In some embodiments, the polymethacrylate is poly(lauryl) methacrylate and the acrylate crosslinker is TMPTA. The weight ratio of the lauryl methacrylate and TMPTA may vary.

In some embodiments, the weight ratio is 99:1, 98:2, 98.5:1.5, or more particularly, 99.4:0.4, 99.6 to 0.4, 99.8:0.2 or 99.01.

In some embodiments, the stimuli-responsive polymer does not comprise polyacrylates or polymethacrylates.

In some embodiments, the stimuli-responsive polymer comprises one or more amorphous or semi-crystalline polyurethanes.

In some embodiments, the stimuli-responsive polymer comprises a semicrystalline polyurethane elastomer with segments that include polyether, polyester, polyurethane, polyurethane urea, poly(isoprene), poly(butadiene) or other crystalline segments.

In some embodiments, the stimuli-responsive polymer comprises semicrystalline polyurethane elastomer, either linear or crosslinked, with segments that include polyether, polyester, polyurethane, polyurethane urea, poly(isoprene), poly(butadiene) or other crystalline segments.

In some embodiments, the stimuli-responsive polymer does not comprise polyurethane.

In some embodiments, the stimuli-responsive polymer comprises a polyolefin. In some embodiments, the polyolefin is a polyisoprene.

In some embodiments, the stimuli-responsive polymer comprises at least one polyether, for example. poly(ethylene glycol) (PEG) compounds and acrylated or polyurethane-containing PEG compounds.

In some embodiments, the stimuli-responsive polymer comprises at least one polyepoxy. In some embodiments, the polyepoxy comprises one or more epoxy monomers disclosed herein.

In some embodiments, the stimuli-responsive polymer does not comprise polyepoxy. In some embodiments, the polyepoxy comprises one or more silicone monomers disclosed herein.

In some embodiments, the stimuli-responsive polymer comprises at least one silicone polymer. In some embodiments, the silicone polymer is a high molecular weight, linear siloxane polymer and a highly condensed, silicate tackifying resin.

In some embodiments, the stimuli-responsive polymer does not comprise a silicone polymer.

In some embodiments, the stimuli-responsive polymer comprises at least one synthetic rubber.

In some embodiments, the stimuli-responsive polymer comprises styrene-butadiene rubber (SBR). Examples of SBRs include those used in the manufacture of pressure sensitive tapes, including synthetic elastomers derived from styrene and butadiene. SBRs, whether solvent borne or waterborne suitable for use in the present disclosure include SBRs with varying percent of bound styrene, average molecular weight and its distribution, and the presence of functional groups introduced during polymerization. Molecular weights of SBRs range from 10 to 1,000,000 g/mol, more specifically 25,000 to 750,000 g/more, more specifically 50,000 to 500,000 g/mol. SBRs generally exhibit low water uptake, less than 1 wt % water, more specifically less than 0.5 wt % water, more specifically 0.1 wt % water, more specifically less than 0.05 wt % water.

In some embodiments, the stimuli-responsive polymer does not comprise synthetic rubber.

In some embodiments, the stimuli-responsive polymer comprises thiol monomers. Exemplary thiol monomers include, but are not limited to, 3-Mercaptopropionic acid; Thioglycolic acid; 3-Mercapto-1-propanol; 2-Mercaptoethanol; 2-(2-Mercaptoethoxy)ethanol; 2-(2-Mercaptopropionylamino)ethanol; 2-(2-Mercaptosuccinyl)ethyl acrylate; 3-(2-Mercaptopropionylamino)propionic acid; 3-(Mercaptopropyl)trimethoxysilane; 2,2′-(Ethylenebis(thio))diethanol; 3-Mercaptopropyltrimethoxysilane; 3-Mercaptopropylmethyldimethoxysilane; 3-(2,2-Dithiobis(ethylthio)propionylamino)propionic acid; 3,6,9-Trioxadecanethiol; 3-Mercapto-1,2-propanediol; 2,2′-Dithiodiethanol; N-Acetyl-L-cysteine; L-Cysteine; 2-(2-Mercaptoethyl)pyridine; 4-(2-Mercaptoethyl)morpholine; 3-Mercapto-1,2,4-triazole; Thiophenol; Pentaerythritol tetrakis(3-mercaptopropionate) (PETMP); Trimethylolpropane tris(3-mercaptopropionate) (TMPMP); Triethanolamine tris(3-mercaptopropionate) (TEAMP); Tris(2-hydroxycthyl) isocyanurate tris(3-mercaptopropionate) (THEICMP); Bis(3-mercaptopropyl) sulfide (BMPS); 1,2-ethanedithiol (EDT); 1,3-propanedithiol; 1,4-butanedithiol; 1,6-hexanedithiol; 1,8-octanedithiol and combinations thereof.

In some embodiments, the stimuli-responsive polymer comprises linear or crosslinked polymers that include liquid crystalline polymers with thermal transitions in the range of −10 °C to 50 °C, more particularly 0 °C to 40 °C, more particularly 5 °C to 35 °C, more particularly 5 °C to 20 °C. Liquid crystalline polymer compositions suitable for use include thiol-ene and thiol-acrylate polymers prepared via base catalyzed Michael addition or free radical polymerization processes, including those prepared from thiol building blocks such as 1,6-hexanedithiol, 1,8-octanedithol, 1,10-decanedithiol, 1,12-dodecanedithiol, hexanediol diacrylate, octanediol diacrylate, decanediol diacrylate and diacrylate species containing mesogens such as RM105—4-(6-Acryloyloxyhexyloxy)-benzoic acid (4-cyanophenyl ester), RM 23—4-Methoxyphenyl 4-((6-(acryloyloxy)hexyl)oxy)benzoate, CB3A—3-[(3′-Cyanobiphenyl-3-yl)oxy]propylacrylate. Di-acrylate mesogens include but are not limited to RM 257-4-(3-acryloyoxy-propyloxy) benzoic acid 2-methyl-1,4-phenylene ester, RM 82-1,4-Bis[4-(6-acryloyloxyhexyloxy)benzoyloxy]-2-methylbenzene

In some embodiments, free-radical polymerization can be used to crosslink acrylate-functionalized liquid crystal networks. Click chemistry reactions such as Michael addition reactions may also be used to incorporate soft flexible segments in between mesogenic monomers to decrease Tg and enable elastomeric behavior at ambient conditions. In some embodiments, dithiols can be used as flexible spacers and include but are not limited to: ethane dithiol, propane dithiol, or any other dithiol with an all-carbon backbone, 2,2′-(ethylenedioxy)diethanethiol or any other dithiol with a polyethylene glycol backbone, 1,4-benzenedithiol, 4,4′-biphenyldithiol, ethylene bis(thioglycolate), glycol dimercaptopropionate.

In some embodiments, in addition to thiols, amine-functionalized monomers may be used in a similar fashion. For example, n-butylamine can be used as a flexible chain extender or spacer for mesogenic monomers. Using a Michael addition catalyst such as triethyl amine or dipropyl amine, acrylate-functionalized mesogenic oligomers can be created by combining a non-stoichiometric ratio of diacrylate mesogens to dithiol monomers or diacrylate mesogens to di-functional amines. In either case, an excess of acrylate functional groups is optionally preferred. These oligomers can then be photo-crosslinked to form an LCE network.

In some embodiments, LCEs can be synthesized in a one-pot manner by utilizing thiol or amine functionalized crosslinkers with a functionality of 2 or greater. This one-pot approach can be used with both free-radical and Michael-addition polymerization methods. Examples include but are not limited to pentaerythritol tetramercaptoacetate (PETMA), trimethylolpropane tri(3-mercaptopropionate), pentaerythritol tetra(3-mercaptopropionate), di-pentaerythritol tetra(3-mercaptopropionate)

In some embodiments, the stimuli-responsive polymer comprises linear or crosslinked polymers that include silicone polymers, for example, a high molecular weight, linear siloxane polymer and a highly condensed, silicate tackifying resin. Tackifying resins or tackifiers include low-molecular weight compounds with high glass transition temperature used in formulating adhesives to increase the tack, the stickiness of the surface of the adhesive. Tackifiers include resins (e.g., rosins and their derivates, terpenes and modified terpenes, aliphatic, cycloaliphatic and aromatic resins (C5 aliphatic resins, C9 aromatic resins, and C5/C9 aliphatic/aromatic resins), hydrogenated hydrocarbon resins, and their mixtures, terpenephenol resins (TPR, used often with ethylene-vinyl acetate adhesives)), novolacs. Silicone rubber-based pressure-sensitive adhesives suitable for use include special tackifiers based on “MQ” silicate resins, composed of a monofunctional trimethyl silane (“M”) reacted with quadrafunctional silicon tetrachloride (“Q”).

In some embodiments, the stimuli-responsive polymer exhibits one or more glass transition (Tg), crystallization temperature (Tc), melting temperature (Tm) or other thermal transition ranging from −100 to 100 °C or about −40 °C, about −30 °C, about −20 °C, about −10 °C, about 10 °C, about 0 °C, about 10 °C, about 15 °C, about 20 °C, about 25 °C, about 30 °C, about 35 °C, about 40 °C, about 45 °C, about 50 °C, about 55 °C, about 60 °C, or about 65 °C or more as measured by differential scanning calorimetry (DSC) Tg, Tc, Tm peak inflection point or dynamic mechanical analysis loss modulus or tan delta peak at 1 Hz.

In some embodiments, the stimuli-responsive polymer is a compostable, bio-based or degradable adhesive prepared, for example from plasticized polycaprolactone or plasticized poly(lactic acid). In some embodiments, plasticization is carried out to lower glass transition or increase tack.

In some embodiments, the stimuli-responsive polymer is suitable for use in food grade applications such as stickers used for produce or fruit.

In some embodiments, the one or more polymers of the stimuli-responsive polymer are selected from, for example, polyacrylates, polymethacrylates, polyurethanes, polyolefins, polyethers, silicones, polyepoxies, synthetic rubbers or other adhesives suitable for use with human skin, including derivatives, copolymers, and mixtures thereof.

In some embodiments, the stimuli-responsive polymer has a glass transition temperature (Tg) from 0 °C to 50 °C, such as, for example, from 0 °C to 40 °C, from 0 °C to 30 °C, from 0 °C to 20 °C, from 0 °C to 10 °C from 5 °C to 50 °C, from 5 °C to 40 °C, from 5 °C to 30 °C, from 5 °C to 20 °C, from 5 °C to 10 °C, from 10 °C to 50 °C, from 10 °C to 40 °C, from 10 °C to 30 °C, from 10 °C to 20 °C, from 20 °C to 50 °C from 20 °C to 40 °C, from 20 °C to 30 °C, from 30 °C to 50 °C, from 30 °C to 40 °C, or from 40 °C to 50 C.

In some embodiments, the stimuli-responsive polymer has a glass transition below ambient temperature, such as, for example, a glass transition from −90 °C to 20 °C, from −90 °C to −80 °C, from −80 °C to −70 °C, from −70 °C to −60 °C, from −60 °C to −50 °C, from −50 °C to −40 °C, from −40 °C to −30 °C, from −30 °C to −20 °C, from −20 °C to −10 °C, from −10 °C to 0 °C, from 0 °C to 10 °C, or from 20 °C to 20 C.

In some embodiments, the stimuli-responsive polymer has a crystalline melt transition below ambient temperature, such as, for example, a crystalline melt transition from −90 °C to 20 °C, from −90 °C to −80 °C, from −80 °C to −70 °C, from −70 °C to −60 °C, from −60 °C to −50 °C, from −50 °C to −40 °C, from −40 °C to −30 °C, from −30 °C to −20 °C, from −20 °C to −10 °C, from −10 °C to 0 °C, from 0 °C to 10 °C, or from 20 °C to 20 °C.

In some embodiments, the stimuli-responsive polymer is a linear or branched crosslinked polymer network wherein the crosslinked polymer network contains 10 or more ester to thiolester linkages which hydrolyze in the presence of an added base or a compound containing a thiol, or a compound containing an amino wherein hydrolysis results in dissolution of the adhesive and delamination from penile skin.

In some embodiments, the stimuli-responsive polymer may be a linear or branched polymer comprising the reaction product by free radical addition polymerization of mono-, di-try-tetra-, penta, and hexafunctional thiol-ene constituents including triallyl isocyanurate pentaerythritol tetrakis (3-mercaptopropionate) or any of the thiol-ene monomeric constituents disclosed herein. In some embodiments, thiol-ene adhesives may exhibit stimuli-responsive adhesive behavior upon cooling below its glass transition. In some embodiments, thiol-ene stimuli-responsive polymer may exhibit chemically-responsive adhesive behavior such as oxidation of thioether linkages by common oxidizing agents such as hydrogen peroxide to form reversibly clearable disulfide linkages.

In some embodiments, the stimuli-responsive polymer may be a linear, brush, star, dendritic, or branched polymer with weight average molecular weight (Mw) of approximately 1 kDa, about 5 kDa, about 10 kDa, about 15 kDa, about 20 kDa, about 30 kDa, about 50 kDa, about 75 kDa, about 90 kda, about 100 kDa, about 110 kDa, about 200 kDa, about 300 kDa, about 400 kDa, about 500 kDa, or about 1000 kDa or more.

In some embodiments, the linear and crosslinked polymers comprise (i) a main chain; (ii) at least one side chain; (iii) crosslinking; and (iv) additives. The main chain (i), side chain (ii) and/or crosslinkers (iii) may comprise one or more monomers.

In some embodiments, the monomers comprising (i), (ii) and (iii) may be the same monomers or different monomers.

In some embodiments, the one or more linear or crosslinked polymers are petroleum-based.

In some embodiments, the one or more linear or crosslinked polymers are bio-based, in whole or in part.

In some embodiments, the stimuli-responsive polymer is a crosslinked polymer that provides a semi-interpenetrating network or is an interpenetrating network.

The amount of stimuli-responsive polymer present in the adhesive (e.g., medical adhesive) composition may vary. In some embodiments, the stimuli-responsive polymer comprises at least 70 wt % of the adhesive (e.g., medical adhesive) composition, such as, for example, at least about 80 wt %, at least 90 wt %, or at least 95 wt %.

In some embodiments, the stimuli-responsive polymer comprises at least 90 wt % of the adhesive (e.g., medical adhesive) composition, such as, for example, at least 91 wt %, at least 92 wt %, at least 93 wt %, at least 94 wt %, at least 95 wt %, at least 96 wt %, at least 97 wt %, at least 98 wt %, or at least 99 wt %.

In some embodiments, the stimuli-responsive polymer does not comprise an acrylic-based adhesive which contains an unreacted polyol plasticizer.

In some embodiments, the adhesive (e.g., medical adhesive) composition does not comprise an acrylic pressure-sensitive adhesive and (i) an elastomer with a tackifying resin or (b) a thermoplastic elastomer. In some embodiments, the stimuli-responsive polymer does not comprise a hydrophobic polyoxyalkylene-based adhesive derived from poly(ethylene glycol) prepared in the presence of a plasticizer.

In some embodiments, the stimuli-responsive polymer does not comprise an acrylic pressure-sensitive adhesive and (i) an elastomer with a tackifying resin or (b) a thermoplastic elastomer. In some embodiments, the adhesive (e.g., medical adhesive) composition does not comprise an acrylic pressure-sensitive adhesive and (i) an elastomer with a tackifying resin or (b) a thermoplastic elastomer.

### 4.2.1.2.1 Monomers

In some embodiments, the stimuli-responsive polymer, or optionally, one or more additional polymers, is formed from (i.e., comprises) monomers selected from acrylate monomers, methacrylate monomers, vinyl ether monomer, allyl monomers, thiol monomers, epoxy monomers, amine monomers, electron rich monomers, electron poor monomers, lactam monomers, lactone monomers, alcohol monomers, carboxylic acid monomers, isocyanate monomers, Diels-Alder monomers, ring opening metathesis monomers, or a combination thereof.

In some embodiments, the stimuli-responsive polymer, or optionally, one or more additional polymers, comprises acrylate monomers. In some embodiments, the acrylate monomers are C6-C30 alkyl acrylate monomers, such as, for example, C8-C30 alkyl acrylate monomers. C8-C20 alkyl acrylate monomers, C8-C16 alkyl acrylate monomers, C8-C12 alkyl acrylate monomers, C12-C30 alkyl acrylate monomers, C12-C20 alkyl acrylate monomers, or C12-C16 alkyl acrylate monomers.

Exemplary acrylate monomers include, but are not limited to, octyl acrylate, nonyl acrylate, decyl acrylate, undecyl acrylate, dodecyl acrylate, tridecyl acrylate, tetradecyl acrylate, pentadecyl acrylate, hexadecyl acrylate, heptadecyl acrylate, octadecyl acrylate, nonadecyl acrylate, eicosyl acrylate, heneicosyl acrylate, docosyl acrylate, tricosyl acrylate, tetracosyl acrylate, pentacosyl acrylate, hexacosyl acrylate, heptacosyl acrylate, octacosyl acrylate, nonacosyl acrylate, triacontyl acrylate, methyl acrylate, ethyl acrylate, Butyl acrylate, 2-ethylhexyl acrylate, isobutyl acrylate, Methoxyethyl acrylate, Hydroxyethyl acrylate, Hydroxypropyl acrylate, Ethoxylated hydroxyethyl acrylate, Glycidyl acrylate, Methacrylic acid, Methyl methacrylate, Ethyl methacrylate, Butyl methacrylate, 2-Hydroxyethyl methacrylate, Cyclohexyl methacrylate, Poly(ethylene glycol) methacrylate, Poly(ethylene glycol) diacrylate, Poly(ethylene glycol) dimethacrylate, Trimethylolpropane triacrylate, Triethylene glycol diacrylate, Tetraethylene glycol diacrylate, Neopentyl glycol diacrylate, Diethylene glycol diacrylate, Dipentaerythritol hexaacrylate, Ethoxylated trimethylolpropane triacrylate, Propoxylated glycerol triacrylate, Stearyl acrylate, Lauryl acrylate, Isodecyl acrylate, Acrylic acid, Ethylene glycol diacrylate (EGDA), Triethylene glycol diacrylate (TEGDA), Propylene glycol diacrylate (PGDA), Butanediol diacrylate (BDDA), Neopentyl glycol diacrylate (NPGDA). Pentaerythritol tetraacrylate (PETA), 1,4-Butanediol diacrylate (BDA), Di(trimethylolpropane) tetraacrylate (DTMPTA), Bisphenol A ethoxylate diacrylate (BPAEDA), Ethoxylated bisphenol A diacrylate (EBPA), Decanediol diacrylate, Polyethylene glycol diacrylate (PEGDA), Trimethylolpropane triacrylate (TMPTA), Diethylene glycol diacrylate (DEGDA), and 1,6-Hexanediol diacrylate (HDDA), Trimethylolpropane triacrylate (TMPTA). Tripropylene glycol diacrylate (TPGDA), Pentaerythritol triacrylate (PETA), Dipentaerythritol pentaacrylate (DPEPA), Tris(2-hydroxyethyl) isocyanurate triacrylate (THEIC-TA), Triethylene glycol dimethacrylate (TEGDMA), Triallyl isocyanurate (TAIC), Triethylene glycol diacrylate (TEGDA), Ethoxylated trimethylolpropane triacrylate (ETMPTA), Triallyl cyanurate (TAC), and combinations thereof.

In certain embodiments, the stimuli-responsive polymer, or optionally, one or more additional polymers, comprises acrylate monomers selected from octyl acrylate, nonyl acrylate, decyl acrylate, undecyl acrylate, dodecyl acrylate, tridecyl acrylate, tetradecyl acrylate, pentadecyl acrylate, hexadecyl acrylate, heptadecyl acrylate, octadecyl acrylate, nonadecyl acrylate, eicosyl acrylate, heneicosyl acrylate, docosyl acrylate, tricosyl acrylate, tetracosyl acrylate, pentacosyl acrylate, hexacosyl acrylate, heptacosyl acrylate, octacosyl acrylate, nonacosyl acrylate, triacontyl acrylate, and combinations thereof.

In some embodiments, the acrylate monomer is TMPTA.

In some embodiments, the stimuli-responsive polymer, or optionally, one or more additional polymers, comprises at least 10 wt % arylate monomers, such as, for example, at least 50 wt %, at least 75 wt %, or at least 90 wt %. In some embodiments, the stimuli-responsive polymer comprises at least 95 wt % acrylate monomers, e.g., at least 96 wt %, at least 97 wt %, at least 98 wt %, or at least 99 wt %.

In some embodiments, the stimuli-responsive polymer, or optionally, one or more additional polymers, comprises methacrylate monomers. In some embodiments, the methacrylate monomers are C6-C30 alkyl methacrylate monomers, such as, for example, C8-C30 alkyl methacrylate monomers, C8-C20 alkyl methacrylate monomers. C8-C16 alkyl methacrylate monomers, C8-C12 alkyl methacrylate monomers, C12-C30 alkyl methacrylate monomers, C12-C20 alkyl methacrylate monomers, or C12-C16 alkyl methacrylate monomers.

Exemplary methacrylate monomers include, but are not limited to, octyl methacrylate, nonyl methacrylate, decyl methacrylate, undecyl methacrylate, dodecyl methacrylate, tridecyl methacrylate, tetradecyl methacrylate, pentadecyl methacrylate, hexadecyl methacrylate, heptadecyl methacrylate, octadecyl methacrylate, nonadecyl methacrylate, eicosyl methacrylate, heneicosyl methacrylate, docosyl methacrylate, tricosyl methacrylate, tetracosyl methacrylate, pentacosyl methacrylate, hexacosyl methacrylate, heptacosyl methacrylate, octacosyl methacrylate, nonacosyl methacrylate, triacontyl methacrylate, Ethyl methacrylate, Butyl methacrylate, 2-Hydroxyethyl methacrylate, Cyclohexyl methacrylate, Isobornyl methacrylate, Stearyl methacrylate, Lauryl methacrylate, Isodecyl methacrylate, Tetrahydrofurfuryl methacrylate, Glyceryl methacrylate, Trimethylolpropane trimethacrylate, Trimethylolpropane triacrylate, Pentaerythritol triacrylate, Pentaerythritol tetramethacrylate, Poly(ethylene glycol) monomethyl ether methacrylate, Poly(ethylene glycol) monomethyl ether acrylate, Poly(ethylene glycol) diacrylate, Poly(ethylene glycol) dimethacrylate, Poly(ethylene glycol) monoacrylate, Ethoxylated bisphenol A dimethacrylate, Ethoxylated bisphenol A diacrylate, Ethoxylated trimethylolpropane triacrylate, Hydroxypropyl methacrylate, Methacrylic acid, Acryloyloxyethyltrimethylammonium chloride, Diethylaminoethyl methacrylate, Butylaminoethyl methacrylate, N,N-dimethylaminoethyl methacrylate, Methacryloyloxyethyl phthalate, Cyclopropyl methacrylate, and combinations thereof.

In some embodiments, the stimuli-responsive polymer, or optionally, one or more additional polymers, comprises methacrylate monomers selected from octyl methacrylate, nonyl methacrylate, decyl methacrylate, undecyl methacrylate, dodecyl methacrylate, tridecyl methacrylate, tetradecyl methacrylate, pentadecyl methacrylate, hexadecyl methacrylate, heptadecyl methacrylate, octadecyl methacrylate, nonadecyl methacrylate, eicosyl methacrylate, heneicosyl methacrylate, docosyl methacrylate, tricosyl methacrylate, tetracosyl methacrylate, pentacosyl methacrylate, hexacosyl methacrylate, heptacosyl methacrylate, octacosyl methacrylate, nonacosyl methacrylate, triacontyl methacrylate, and combinations thereof. In some embodiments, the stimuli-responsive polymer comprises undecyl methacrylate monomers, i.e., lauryl methacrylate monomers. In some embodiments, the stimuli-responsive polymer comprises octadecyl methacrylate monomers, i.e., stearyl methacrylate monomers.

In some embodiments, the stimuli-responsive polymer, or optionally, one or more additional polymers, comprises at least 10 wt % methacrylate monomers, such as, for example, at least 50 wt %, at least 75 wt %, or at least 90 wt %. In some embodiments, the stimuli-responsive polymer comprises at least 95 wt % methacrylate monomers, e.g., at least 96 wt %, at least 97 wt %, at least 98 wt %, or at least 99 wt %.

In some embodiments, the stimuli-responsive polymer, or optionally, one or more additional polymers, comprises vinyl ether monomers. Exemplary vinyl ether monomers include, but are not limited to, divinyl ether of ethylene glycol, divinyl ether of diethylene glycol, divinyl ether of triethylene glycol, divinyl ether of polyethylene glycol (DVE-PEG), divinyl ether of polypropylene glycol (DVE-PPG), Divinyl ether of poly(ethylene glycol) methyl ether (DVE-PEGME), divinyl ether of poly(ethylene glycol) butyl ether (DVE), Divinyl ether of poly(ethylene glycol) phenyl ether (DVE-PEGPhE), Divinyl ether of glycerol (DVE-Gly), Divinyl ether of 1,4-cyclohexanedimethanol (DVE-CHDM), Divinyl ether of neopentyl glycol (DVE-NPG), and combinations thereof.

In some embodiments, the stimuli-responsive polymer, or optionally, one or more additional polymers, comprises allyl monomers. Exemplary allyl monomers include, but are not limited to, Diallyl phthalate (DAP), Diallyl maleate (DAM), Diallyl succinate (DAS), Diallyl fumarate (DAF). Diallyl adipate (DAA), Diallyl sebacate (DAS), Diallyl terephthalate (DAT), Diallyl isophthalate (DAI), Diallyl itaconate (DAI), Diallyl carbonate (DAC), Diallyl diglycolate (DADG), Diallyl tris(2-hydroxyethyl) isocyanurate (DATHEIC), Triallyl cyanurate (TAC), Triallyl isocyanurate (TAIC), Triallyl trimellitate (TATM), Triallyl citrate (TAC), Triallyl phosphate (TAP), Triallylamine (TAA), Triallyl cyanide (TACN), Triallyl benzene-1,2,4-tricarboxylate (TABTC), Triallyl trimesate (TATM), Tris(2-hydroxyethyl) isocyanurate triallyl ether (THEIC-TAE), and combinations thereof.

In some embodiments, the stimuli-responsive polymer, or optionally, one or more additional polymers, comprises thiol monomers. Exemplary thiol monomers include, but are not limited to, 3-Mercaptopropionic acid; Thioglycolic acid; 3-Mercapto-1-propanol; 2-Mercaptoethanol; 2-(2-Mercaptoethoxy)ethanol; 2-(2-Mercaptopropionylamino)ethanol; 2-(2-Mercaptosuccinyl)ethyl acrylate; 3-(2-Mercaptopropionylamino)propionic acid; 3-(Mercaptopropyl)trimethoxysilane; 2,2′-(Ethylenebis(thio))diethanol; 3-Mercaptopropyltrimethoxysilane; 3-Mercaptopropylmethyldimethoxysilane; 3-(2,2-Dithiobis(ethylthio)propionylamino)propionic acid; 3,6,9-Trioxadecanethiol; 3-Mercapto-1,2-propanediol; 2,2′-Dithiodiethanol; N-Acetyl-L-cysteine; L-Cysteine; 2-(2-Mercaptoethyl)pyridine; 4-(2-Mercaptoethyl)morpholine; 3-Mercapto-1,2,4-triazole; Thiophenol; Pentaerythritol tetrakis(3-mercaptopropionate) (PETMP); Trimethylolpropane tris(3-mercaptopropionate) (TMPMP); Triethanolamine tris(3-mercaptopropionate) (TEAMP); Tris(2-hydroxyethyl) isocyanurate tris(3-mercaptopropionate) (THEICMP); Bis(3-mercaptopropyl) sulfide (BMPS); 1,2-ethanedithiol (EDT); 1,3-propanedithiol; 1,4-butanedithiol; 1,6-hexanedithiol; 1,8-octanedithiol and combinations thereof.

In some embodiments, the stimuli-responsive polymer, or optionally, one or more additional polymers, comprises epoxy monomers. Exemplary epoxy monomers include, but are not limited to, Bisphenol A diglycidyl ether (BADGE). Bisphenol F diglycidyl ether (BFDGE), Novolac diglycidyl ether (NGDE), Phenol novolac diglycidyl ether (PNGDE), Cycloaliphatic epoxy resins, Glycidyl ethers of aliphatic alcohols, Glycidyl ethers of aromatic alcohols, Triglycidyl isocyanurate (TGIC), Diglycidyl ether of 1,4-butanediol (BDDGE), Diglycidyl ether of neopentyl glycol (NPGDGE), Diglycidyl ether of propylene glycol (PGDGE), Epoxidized soybean oil (ESO), Epoxidized linseed oil (ELO), Dicyclopentadiene-based epoxy resins, Tetrafunctional epoxy resins, Epoxy phenolic novolac resins, and combinations thereof.

In some embodiments, the stimuli-responsive polymer, or optionally, one or more additional polymers, comprises amine monomers. Exemplary amine monomers include, but are not limited to, Ethylenediamine; Diethylenetriamine; Triethylenetetramine; Tetraethylenepentamine; Polyethyleneimine; Diaminopropane; Diaminobutane; Diaminopentane; Diethylenetriaminepentaacetic acid (DTPA); Tris(2-aminoethyl)amine; N-(2-Aminoethyl)piperazine; N-(3-Aminopropyl)morpholine; N,N-Dimethylaminopropylamine; N,N-Dimethylethylenediamine; 1,3-Diaminopropane; Isophoronediamine; Jeffamine D-230; Jeffamine T-403; Jeffamine M-207; Jeffamine EDR-148; and combinations thereof.

In some embodiments, the stimuli-responsive polymer, or optionally, one or more additional polymers, comprises electron rich monomers. Exemplary electron rich monomers include, but are not limited to, Vinyl ethers (e.g. vinyl methyl ether, vinyl ethyl ether), Vinyl acetate, Allyl alcohol, Allyl amine, N-Methylolacrylamide, N-Methylolmethacrylamide, N-Methylolallylamine, N-Methylolvinylacetamide, Acrolein diethyl acetal, Acrolein diethyl ketal, Diacetone acrylamide, 2-Hydroxyethyl acrylate, 2-Hydroxyethyl methacrylate, 2-Hydroxypropyl acrylate, 2-Hydroxypropyl methacrylate, 2,3-Dihydroxypropyl methacrylate, Glycidyl methacrylate, Glycidyl acrylate, Tetrahydrofurfuryl methacrylate, and N-Vinylpyrrolidone, n-vinylformamide, n-vinyl pyridine, styrene, styrene derivatives, and combinations thereof.

In some embodiments, the stimuli-responsive polymer, or one or more additional polymers, comprises electron poor monomers. Exemplary electron poor monomers include, but are not limited to, Acrylonitrile, Methacrylonitrile, Methyl methacrylate, Acrylic acid, Methacrylic acid, Maleic anhydride, Itaconic acid, Fumaric acid, Acrylamide, Methacrylamide, N-Vinylcarbazole, Vinylidene chloride, Vinyl chloride, Vinyl sulfonic acid, Vinyl acetate, Styrene, Alpha-methylstyrene, Maleimide, N-Phenylmaleimide, and N-Butylmaleimide, maleic anhydride, and combinations thereof.

In some embodiments, the stimuli-responsive polymer, or optionally, one or more additional polymers, comprises lactam monomers. Exemplary lactam monomers include, but are not limited to. Caprolactam, Valerolactam, Enantholactam, Capryllactam, Laurinlactam, Prolactam, Butyrolactam, Methionyl lactam, Methoxyethyl lactam, Methoxyethyl methionyl lactam, Dimethylaminoethyl lactam, Dimethylaminoethyl methionyl lactam, Dimethylaminoethyl acryloyl lactam, Dimethylaminoethyl methacryloyl lactam, N-vinylpyrrolidone, N-methylpyrrolidone, N-ethylpyrrolidone, and combinations thereof.

In some embodiments, the stimuli-responsive polymer, or optionally, one or more additional polymers, comprises lactone monomers. Exemplary lactone monomers include, but are not limited to, β-propiolactone, γ-butyrolactone, 8-valerolactone, ε-caprolactone, ω-pentadecalactone, β-butyrolactone, 8-decalactone, ε-decalactone, γ-decalactone, 8-dodecalactone, γ-dodecalactone, α-methylene-γ-butyrolactone, β-methyl-γ-butyrolactone, β-methyl-γ-valerolactone, γ-hexalactone, and combinations thereof.

In some embodiments, the stimuli-responsive polymer, or optionally, one or more additional polymers, comprises alcohol monomers. Exemplary alcohol monomers include, but are not limited to, Ethylene glycol; Propylene glycol; 1,3-Butanediol; 1,4-Butanediol; 1,5-Pentanediol; 1,6-Hexanediol; 1,10-Decanediol; Neopentyl glycol; Diethylene glycol; Triethylene glycol; Tetraethylene glycol; Polyethylene glycol (PEG); Polypropylene glycol (PPG); Polycaprolactone diol; Polymethylolpropane; Hydroxypivalyl hydroxymethylbutyrate (HPHMB); 1,4-Cyclohexanedimethanol; and combinations thereof.

In some embodiments, the stimuli-responsive polymer, or optionally, one or more additional polymers, comprises carboxylic acid monomers. Exemplary carboxylic acid monomers include, but are not limited to, Adipic acid, Succinic acid, Glutaric acid, Sebacic acid, Malonic acid, Phthalic acid, Isophthalic acid, Terephthalic acid, Fumaric acid, Maleic acid, Itaconic acid, Citric acid, 1,4-Cyclohexanedicarboxylic acid, 1,3-Cyclohexanedicarboxylic acid, Dodecanedioic acid, and combinations thereof.

In some embodiments, the stimuli-responsive polymer, or optionally, one or more additional polymers, comprises isocyanate monomers. Exemplary isocyanate monomers include, but are not limited to, Toluene diisocyanate (TDI), Diphenylmethane diisocyanate (MDI), Hexamethylene diisocyanate (HDI), Isophorone diisocyanate (IPDI), 1,6-Hexamethylene diisocyanate (HMDI), 4,4′-Methylenebis(cyclohexyl isocyanate) (H12MDI), Naphthalene diisocyanate (NDI), 2,4-Toluene diisocyanate (2,4-TDI), 2,6-Toluene diisocyanate (2,6-TDI), Polymethylene polyphenyl isocyanate (PAPI), Desmodur N-100, Desmodur L-75, Desmodur HL, Desmodur H. Desmodur VP, Desmodur Z, and combinations thereof.

In some embodiments, the stimuli-responsive polymer, or optionally, one or more additional comprises Diels-Alder monomers. Exemplary Diels-Alder monomers include, but are not limited to, Maleic anhydride, Furan, Cyclopentadiene, N-phenylmaleimide, Anthracene, N-ethylmaleimide, N-phenylnorbornene, N,N-dimethyl maleimide, 2,5-dimethylfuran, Tetracyanoethylene, Methyl vinyl ketone, and combinations thereof.

In some embodiments, the stimuli-responsive polymer, or optionally, one or more additional polymers, comprises ring opening metathesis monomers. Exemplary ring opening metathesis monomers include, but are not limited to, Norbornene, Dicyclopentadiene (DCPD), Cyclooctene, Tetracyclododecene (TCD), Cyclopentene, Cycloheptene, Cyclohexene, Bicyclo[2.2.1]hept-2-ene, Bicyclo[2.2.2]oct-5-ene, Tricyclo[5.2.1.0(2,6)]dec-8-ene (TCD-Diene), and combinations thereof.

### 4.2.1.2.2 Side Chains

In some embodiments, the stimuli-responsive polymer comprises two or more side chains.

In some embodiments, the stimuli-responsive polymer comprises C6 to C30 side chains or C6 to C30 dangling chain ends. In some embodiments, the C6 to C30 side chains or C6 to C30 dangling chain ends are C6 to C30 alkyl side chains, preferably C12 to C18 alkyl side chains.

In some embodiments, the stimuli-responsive polymers comprises at least 80 wt % side chains, such as, for example, at least 85 wt %, at least 90 wt %, or at least 95 wt %. In some embodiments, the side chains are the same. In some embodiments, the side chains are different.

In some embodiments, the side chain comprises linkages made from monomers selected from acrylate, methacrylate, thiol-acrylate Michael Addition, acrylate amine Michael Addition, epoxy thiol, epoxy amine, polyethylenimine (PEI), thiol-ene, alternating copolymers made from C═C electron poor+C═C electron rich monomers, urethane, urea, acrylamide, methacrylamide, polyester, polycarbonate, polyamide, peptoid, peptide, Diels-alder, lactides and lactams, and ring opening metathesis polymerization or olefin metathesis reactions.

In some embodiments, side chain chemistries include C1-C100 side chain linkages achieved by synthetic pathways disclosed herein.

In some embodiments, stimuli-responsive polymers with C6 to C18 side chains or dangling chain ends are prepared from acrylate, methacrylate, alcohol, carboxylic acid, electron rich alkene, electron poor alkene, epoxy, amine, ROMP, Diels-Alder, Lactone, Lactam, Peptide, Peptoid, acrylamide, methacrylamide, thiol, vinyl and allyl monomers.

In some embodiments, the stimuli-responsive polymer comprises a linear or crosslinked polymer with a side chain that optionally undergoes crystallization and/or melting in the region around body and room temperature, in the range of 0 °C to 50 °C, more particularly in the range of 5 °C to 45 °C, more particularly in the range of 10 °C to 40 °C, more particularly in the range of 15 °C to 35 °C. For clarity, side chain crystallization is optional.

In some embodiments, ranges for the linear or crosslinked polymer range from about 40 to about 100%, more particularly, about 50 to 100%. In some embodiments, the range is from about 40 to about 50%, about 50 to about 60%, about 60 to about 70%, about 70 to about 80%, about 80 to about 90% or about 90% or more or any range or value subsumed therein.

In some embodiments, ranges for secondary side chain optionally crystallizable polymer range from about 0 to about 50%, more particularly 1 to about 49%, more particularly about 2 to about 48%, more particularly about 3 to 47%, more particularly about particularly 5 to about 45% or any additional range or value subsumed therein.

### 4.2.1.2.3 Polyfunctional Crosslinker

In some embodiments, the stimuli-responsive polymer, or optionally, one or more additional polymers, is a crosslinked polymer. Crosslinked polymers are prepared using one or more polyfunctional crosslinkers. Crosslinkers may exhibit functionality greater than n=1 reactive site statistically, for example, statistical averages of n=2, 3, 4, 5, 6, 7, 8, 9, 10 or more reactive sites and may facilitate branching, hyperbranching, interpenetrating networks, semi-interpenetrating networks and generally homogeneous or partially homogeneous and partially heterogeneous or generally heterogeneous networks with respect to phase blending or concentration of crosslink density.

In some embodiments, the polyfunctional crosslinker is selected from a difunctional crosslinker, a trifunctional crosslinker, or a tetrafunctional crosslinker. In some embodiments, the polyfunctional crosslinker is a trifunctional crosslinker.

In some embodiments, the polyfunctional crosslinker is a trifunctional crosslinker. In some embodiments, the trifunctional crosslinker is a trifunctional acrylate crosslinker.

In some embodiments, the polyfunctional crosslinker is selected from poly(ethylene glycol) diacrylate, trimethylolpropane triacrylate (TMPTA); ethoxylated trimethyolpropane triacrylate; pentaerythritol tetraacrylate; ethoxylated pentaeiythritol tetraacrylate; dipentaerythritol hexaacrylate; ethoxylated dipentaerithrotol hexaacrylate; di-, tri-, tetra-, penta-, or hexa-epoxides; polythiols; polyalkenes; tris(2-acryloxyethyl) isocyanurate, ε-caprolactone modified tris(2-acryloxyethyl) isocyanurate, ethoxylated glycerine triacrylate, pentaerythritol triacrylate, and combinations thereof.

In some embodiments, the polyfunctional crosslinker is poly(ethylene glycol) diacrylate with internal repeat units ranging from 1 to 1000 or more, trimethylolpropane triacrylate (TMPTA), ethoxylated trimethyolpropane triacrylate with repeat units ranging from 1 to 1000 or more, pentaerythritol tetraacrylate, ethoxylated pentaerythritol tetraacrylate with repeat units ranging from 1 to 1000 or more, penta and hexafunctional acrylates and ethoxylated versions as described above including dipentaerythritol hexaacrylate and ethoxylated dipentaerythritol hexaacrylate with repeat units ranging from 1 to 1000 or more, di, tri, tetra, penta, hexa more expoxide monomers, polythiols, polyalkenes cured by UV light, visible light, gamma or e-beam irradiation, heat or hydrosilation.

In some embodiments, the polyfunctional crosslinker is selected from trimethylolpropane triacrylate (TMPTA), ethoxylated trimethylolpropane triacrylate, polyethylene glycol diacrylate, pentaerythritol tetraacrylate, ethoxylated pentaerythritol tetraacrylate, hexanediol diacrylate, and combinations thereof. In some embodiments, the polyfunctional crosslinker is trimethylolpropane triacrylate (TMPTA).

In some embodiments, the stimuli-responsive polymer, or optionally, one or more additional polymers, is a crosslinked polymer having a homogeneous crosslinked network. In some embodiments, the stimuli-responsive polymer, or optionally, one or more additional polymers, is a crosslinked polymer having a heterogeneous crosslink network. For example, favorable adhesive failure or delamination from skin, including residual adhesive removal after peeling away of adhesive, may be achieved by heterogeneous crosslinking distribution to concentrate failure sites within network or a “swollen heterogeneous network” formed by polymerization of a crosslinker and a monomer with different reactivity ratios.

In some embodiments, the stimuli-responsive polymer, or optionally, one or more additional polymers, is characterized by low-density crosslinking.

In some embodiments, the crosslinker is incorporated into the stimuli-responsive polymer, or optionally, one or more additional polymers, network uniformly.

In some embodiments, the crosslinker is aggregated within the stimuli-responsive polymer network. This embodiment creates high stress concentration network sites and/or drives rheological behavior and enables energy dissipation (high tan delta and high loss modulus in comparison with those in a more uniform network, to facilitate adhesive failure when desirable or removal from a surface.

The amount of the crosslinker in the stimuli-responsive polymer, or optionally, one or more additional polymers, may vary. In some embodiments, the polymer comprises from 0 to 30 wt % of the crosslinker, such as, for example, from 0.001 to 29 wt %, more particularly 0.005 to 28 wt %, more particularly 0.0075 to 28 wt %, more particularly 0.01 to 27 wt %, more particularly 0.02 to 26 wt %, more particularly 0.05 to 26 wt %, more particularly 0.1 to 25 wt %, more particularly 0.15 to 24 wt %.

In some embodiments, the stimuli-responsive polymer comprises a crosslinker in an amount selected from about 0.1 wt %, about 0.2 wt %, about 0.3 wt %, about 0.4 wt %, about 0.5 wt %, about 0.6 wt %, about 0.7 wt %, about 7.5 wt %, about 0.8 wt %, about 0.9 wt %, about 9.5 wt % or about 1.0 wt % or more, in each case with respect to the weight of the polymer.

In a particular embodiment, the stimuli-responsive polymer comprises a crosslinker in an amount from about 0.1 wt % and about 1 wt %, from about 0.2 wt % to about 0.8 wt %, from about 0.3 wt % to about 0.7 wt %, or from about 0.4 wt % to about 0.6 wt %, in each case with respect to the weight of the polymer.

In some embodiments, the stimuli-responsive polymer comprises from 0.1 wt % to 2.5 wt % polyfunctional crosslinker, such as, for example, from 0.1 wt % to 2 wt %, from 0.1 to 1.5 wt %, from 0.1 wt % to 1 wt %, from 0.1 wt % to 0.9 wt, from 0.1 wt % to 0.8 wt %, from 0.1 wt % to 0.7 wt %, from 0.1 wt % to 0.6 wt %, from 0.1 wt % to 0.5 wt %, from 0.1 wt % to 0.4 wt %, from 0.1 wt % to 0.3 wt % from 0.1 wt % to 0.2 wt %, from 0.2 wt % to 1.5 wt %, from 0.2 wt % to 1 wt %, from 0.3 wt % to 1.5 wt %, from 0.3 wt % to 1 wt %, from 0.4 wt % to 1.5 wt %, from 0.4 wt % to 1 wt %, from 0.5 wt % to 1.5 wt %, from 0.5 wt % to 1 wt %, from 0.6 wt % to 1.5 wt %, from 0.6 wt % to 1 wt %, from 0.7 wt % to 1.5 wt %, from 0.7 wt % to 1 wt %, from 0.8 wt % to 1.5 wt %, from 0.8 wt % to 1 wt %, from 0.9 wt % to 1.5 wt % or from 0.9 wt % to 1 wt %.

In some embodiments, the stimuli-responsive polymer comprises from 0.4 wt % to 0.8 wt % polyfunctional crosslinker, such as, for example, from 0.4 wt % to 0.7 wt %, from 0.4 wt % to 0.6 wt %, from 0.4 wt % to 0.5 wt %, from 0.5 wt % to 0.8 wt %, from 0.5 wt % to 0.7 wt %, from 0.5 wt % to 0.6 wt %, from 0.6 wt % to 0.8 wt %, from 0.6 wt % to 0.7 wt %, or from 0.7 wt % to 0.8 wt %. In some embodiments, the polyfunctional crosslinker is trimethylolpropane triacrylate (TMPTA).

In some embodiment, the weight ratio of the one or more monomers to the one or more polyfunctional crosslinkers is 99:1, 98:2, 97:3, 96:4, 95.3, 94:6, 93.7:92:8, 91:9, 90:10, 89:11, 88:12, 87:13, 86:14; 85:15, 84:16, 83:17, 82:18, 81:19, 80:20, 79:21, 78:22, 77:23, 76:24, 75:25, 74:26, 73:27, 72:28. 71:29, 70:30. 69:31, 68:32, 67:33, 66:34, 65:35, 64:36, 63:37, 62:38, 61:39, 60:40, 59:41, 58:42, 57:43, 56:44, 55:45, 54:46, 53:47, 52:48, 51:49 or 50:50.

In some embodiments, the weight ratio of the one or more monomers to the one or more polyfunctional crosslinkers is from 98:2 to 99.9:0.1, e.g., from 98.5:1.5 to 99.9:0.1; from 99:1 to 99.9:0.1, from 99.1:0.9 to 99.9:0.1, from 99.2:0.8 to 99.9:0.1; from 99.3:0.7 to 99.9:0.1, from 99.4:0.6 to 99.9:0.1, from 99.5:0.5 to 99.9:0.1, from 99.6:0.4 to 99.9:0.1, from 99.7:0.3 to 99.9:0.1, or from 99.8:0.2 to 99.9:0.1.

### 4.2.1.2.4 Embodiments

In some embodiments, the stimuli-responsive polymer comprises one or more methacrylate monomer and one or more acrylate monomer crosslinker. In some embodiments, the weight ratio of the one or more methacrylate monomer to the one or more acrylate monomer crosslinker may vary. In some embodiments, the weight ratio is 99:1, 98:2, 97:3, 96:4, 95.3, 94:6, 93.7:92:8, 91:9, 90:10, 89:11, 88:12m 87:13, 86:14; 85:15, 84:16, 83:17, 82:18, 81:19, 80:20, 79:21, 78:22, 77:23, 76:24, 75:25, 74:26, 73:27. 72:28, 71:29. 70:30, 69:31, 68:32, 67:33, 66:34, 65:35, 64:36, 63:37, 62:38, 61:39, 60:40, 59:41, 58:42, 57:43, 56:44, 55:45, 54:46, 53:47, 52:48, 51:49 or 50:50.

In some embodiments, the stimuli-responsive polymer comprises one or more C6-C30 alkyl methacrylate monomers and one or more C6-C30 alkyl acrylate monomers. The weight ratio of the one or more C8-C30 alkyl methacrylate monomers to the one or more C6-C30 alkyl acrylate monomers may vary. In some embodiments, the weight ratio is 99:1, 98:2, 97:3, 96:4, 95.3, 94:6, 93.7:92:8, 91:9, 90:10, 89:11, 88:12m 87:13, 86:14; 85:15, 84:16, 83:17, 82:18, 81:19, 80:20, 79:21, 78:22, 77:23, 76:24, 75:25, 74:26, 73:27, 72:28, 71:29, 70:30, 69:31, 68:32, 67:33, 66:34, 65:35, 64:36, 63:37, 62:38, 61:39, 60:40, 59:41, 58:42, 57:43, 56:44, 55:45, 54:46, 53:47, 52:48, 51:49 or 50:50.

In some embodiments, the stimuli-responsive polymer comprises both C6-C30 alkyl acrylate monomers and C6-C30 alkyl methacrylate monomers. The molar ratio of the components according to this embodiment may vary consistent with stoichiometric calculations from mass ratio equivalents.

In some embodiments, the stimuli-responsive polymer comprises poly(lauryl methacrylate), i.e., poly(dodecyl methacrylate), crosslinked with one or more polyfunctional crosslinkers. In some embodiments, the weight ratio of the lauryl methacrylate to the one or more polyfunctional crosslinkers is from 98:2 to 99.9:0.1, e.g., from 98.5:1.5 to 99.9:0.1; from 99:1 to 99.9:0.1, from 99.1:0.9 to 99.9:0.1, from 99.2:0.8 to 99.9:0.1; from 99.3:0.7 to 99.9:0.1, from 99.4:0.6 to 99.9:0.1, from 99.5:0.5 to 99.9:0.1, from 99.6:0.4 to 99.9:0.1, or from 99.7:0.3 to 99.9:0.1, from 99.8:0.2 to 99.9:0.1.

In some embodiments, the stimuli-responsive polymer comprises poly(lauryl methacrylate) crosslinked with one or more trifunctional crosslinkers. In some embodiments, the weight ratio of the lauryl methacrylate to the one or more trifunctional crosslinkers is from 98:2 to 99.9:0.1, e.g., from 98.5:1.5 to 99.9:0.1; from 99:1 to 99.9:0.1, from 99.1:0.9 to 99.9:0.1, from 99.2:0.8 to 99.9:0.1; from 99.3:0.7 to 99.9:0.1, from 99.4:0.6 to 99.9:0.1, from 99.5:0.5 to 99.9:0.1, from 99.6:0.4 to 99.9:0.1, or from 99.7:0.3 to 99.9:0.1, or from 99.8:0.2 to 99.9:0.1.

In some embodiments, the stimuli-responsive polymer comprises poly(lauryl methacrylate) crosslinked with an acrylate crosslinker. In some embodiments, the weight ratio of the lauryl methacrylate to the one or more acrylate crosslinkers is from 98:2 to 99.9:0.1, e.g., from 98.5:1.5 to 99.9:0.1; from 99:1 to 99.9:0.1, from 99.1:0.9 to 99.9:0.1, from 99.2:0.8 to 99.9:0.1; from 99.3:0.7 to 99.9:0.1, from 99.4:0.6 to 99.9:0.1, from 99.5:0.5 to 99.9:0.1, from 99.6:0.4 to 99.9:0.1, or from 99.7:0.3 to 99.9:0.1, or from 99.8:0.2 to 99.9:0.1.

In some embodiments, the stimuli-responsive polymer comprises poly(lauryl methacrylate) crosslinked with a trifunctional acrylate crosslinker. In some embodiments, the weight ratio of the lauryl methacrylate to the one or more trifunctional acrylate crosslinkers is from 98:2 to 99.9:0.1, e.g., from 98.5:1.5 to 99.9:0.1; from 99:1 to 99.9:0.1, from 99.1:0.9 to 99.9:0.1, from 99.2:0.8 to 99.9:0.1; from 99.3:0.7 to 99.9:0.1, from 99.4:0.6 to 99.9:0.1, from 99.5:0.5 to 99.9:0.1, from 99.6:0.4 to 99.9:0.1, or from 99.7:0.3 to 99.9:0.1, or from 99.8:0.2 to 99.9:0.1.

In some embodiments, the stimuli-responsive polymer comprises poly(lauryl methacrylate) crosslinked with one or more polyfunctional crosslinkers selected from poly(ethylene glycol) diacrylate, trimethylolpropane triacrylate; ethoxylated trimethyolpropane triacrylate; pentaerythritol tetraacrylate; ethoxylated pentaerythritol tetraacrylate; dipentaerythritol hexaacrylate; ethoxylated dipentaerithrotol hexaacrylate; di-, tri-, tetra-, penta-, or hexa-epoxides; polythiols; polyalkenes; tris(2-acryloxyethyl) isocyanulate, ε-caprolactone modified tris(2-acryloxyethyl) isocyanurate, ethoxylated glycerine triacrylate, ethoxylated glycerine triacrylate, and pentaerythritol triacrylate. In some embodiments, the weight ratio of the lauryl methacrylate to the one or more polyfunctional crosslinkers is from 98:2 to 99.9:0.1, e.g., from 98.5:1.5 to 99.9:0.1; from 99:1 to 99.9:0.1, from 99.1:0.9 to 99.9:0.1, from 99.2:0.8 to 99.9:0.1; from 99.3:0.7 to 99.9:0.1, from 99.4:0.6 to 99.9:0.1, from 99.5:0.5 to 99.9:0.1, from 99.6:0.4 to 99.9:0.1, or from 99.7:0.3 to 99.9:0.1, or from 99.8:0.2 to 99.9:0.1.

In some embodiments, the stimuli-responsive polymer comprises poly(lauryl methacrylate) crosslinked with one or more polyfunctional crosslinkers selected from trimethylolpropane triacrylate, ethoxylated trimethylolpropane triacrylate, polyethylene glycol diacrylate, pentaerythritol tetraacrylate, ethoxylated pentaerythritol tetraacrylate, hexanediol diacrylate, and combinations thereof. In some embodiments, the weight ratio of the lauryl methacrylate to the one or more polyfunctional crosslinkers is from 98:2 to 99.9:0.1, e.g., from 98.5:1.5 to 99.9:0.1; from 99:1 to 99.9:0.1, from 99.1:0.9 to 99.9:0.1, from 99.2:0.8 to 99.9:0.1; from 99.3:0.7 to 99.9:0.1, from 99.4:0.6 to 99.9:0.1, from 99.5:0.5 to 99.9:0.1, from 99.6:0.4 to 99.9:0.1, or from 99.7:0.3 to 99.9:0.1, or from 99.8:0.2 to 99.9:0.1.

In some embodiments, the stimuli-responsive polymer comprises poly (lauryl methacrylate) crosslinked with trimethylolpropane triacrylate. In some embodiments, the weight ratio of the lauryl methacrylate to the trimethylolpropane triacrylate is from 98:2 to 99.9:0.1, e.g., from 98.5:1.5 to 99.9:0.1; from 99:1 to 99.9:0.1, from 99.1:0.9 to 99.9:0.1, from 99.2:0.8 to 99.9:0.1; from 99.3:0.7 to 99.9:0.1, from 99.4:0.6 to 99.9:0.1, from 99.5:0.5 to 99.9:0.1, from 99.6:0.4 to 99.9:0.1, or from 99.7:0.3 to 99.9:0.1, or from 99.8:0.2 to 99.9:0.1.

In some embodiments, the adhesive comprises poly(lauryl methacrylate) and TMPTA, wherein the TMPTA crosslinks the lauryl methacrylate. The weight ratio of the components according to this embodiment may vary. In some embodiments, the weight ratio is 99:1, or more particularly, about 99.4:0.6, about 99.6:0.4 or about 99.8:0.2.

### 4.2.2. Additives

In some embodiments, the adhesive (e.g., medical adhesive) composition further comprises one or more additives. Exemplary additives include tackifiers, plasticizers, pigments, fillers, fluorescents, flow agents, wetting agents, surfactants, anti-foaming agents, rheology modifiers, colorants, permeation enhancers, stabilizers, antioxidants, and combinations thereof. In some embodiments, adhesion may be enhanced or decreased through the addition of an additive.

Exemplary plasticizers include, are not limited to, Glyceryl triacetate (triacetin), Glyceryl monooleate (GMO), Glyceryl monostearate (GMS), Glyceryl tristearate (tristearin), Glyceryl tributyrate (tributyrin), Glyceryl tripropionate (triproprionin), Glyceryl trioleate (triolein), Glyceryl dilaurate (GDL), Glyceryl dimyristate (GDM), Glyceryl distearate (GDS), Diethylhexyl phthalate (DEHP), Diisononyl phthalate (DINP), Dibutyl phthalate (DBP), Diisodecyl phthalate (DIDP), Butyl benzyl phthalate (BBP), Dimethyl phthalate (DMP), Di-n-octyl phthalate (DnOP), Diisobutyl phthalate (DIBP), Diethyl phthalate (DEP), Dicyclohexyl phthalate (DCHP), Methyl decanoate, Ethyl decanoate, Propyl decanoate, Isopropyl decanoate, Butyl decanoate, Isobutyl decanoate, Pentyl decanoate, Hexyl decanoate, Heptyl decanoate, Octyl decanoate, Decyl decanoate, and C1-C20 anoates on either side of ester.

In some embodiments, a plasticizer may be added to lower glass transition of a polymer to tune adhesive regime. Plasticizers suitable for use include glycerol, 1,-butanol 1-octanol, stearic acid, n-butyl stearate, poly(ethylene glycol), Mw varying from 100 to 200 to 400 to 1000 to 2000 to 4000 to 10000 daltons or more, water, various organic solvents, 1-decanoate, and 1-octanoate.

In some embodiments, the additives are stimuli-responsive. For example, an additive such as poly(ethylene glycol) Mw 400 daltons or glycerol could be used in blending ratios from 1 wt % 90 wt % with an adhesive layer constituent such as poly(n-dimethyl acylamide), such that the additive plasticizes an adhesive layer above the crystallization temperature of the additive and no longer plasticizes the adhesive layer below the crystallization temperature of the additive.

In some embodiments, stimuli-responsive additives may exhibit crystallization temperate, glass transition, or other thermal transition in the range of 0 °C to 50 °C, more particularly 5 °C to 40 °C, more particularly 10 °C to 30 °C, and further particularly 12 °C to 25 °C.

Exemplary tackifiers include, but are not limited to, Rosin esters, Hydrocarbon resins, Terpene resins, Styrene resins, Polyterpene resins, Coumarone-indene resins, Phenolic resins, Tall oil rosin, Aliphatic resins, and Aromatic resins.

Exemplary tackifying resins or tackifiers include low-molecular weight compounds with high glass transition temperature used in formulating adhesives to increase the tack, the stickiness of the surface of the adhesive. In some embodiments, tackifiers include resins (e.g., rosins and their derivates, terpenes and modified terpenes, aliphatic, cycloaliphatic and aromatic resins (C5 aliphatic resins, C9 aromatic resins, and C5/C9 aliphatic/aromatic resins), hydrogenated hydrocarbon resins, and their mixtures, terpene-phenol resins (TPR, used often with ethylene-vinyl acetate adhesives)), novolacs. Silicone rubber-based pressure-sensitive adhesives suitable for use in the present disclosure include special tackifiers based on “MQ” silicate resins, composed of a monofunctional trimethyl silane (“M”) reacted with quadrafunctional silicon tetrachloride (“Q”).

In some embodiments, the tackifier or plasticizer is selected from glyceryl, phthalates, polyethylene glycol derivatives with molecular weights ranging from 1 to 1000 or more repeat units, and C10-C40 linear or branched wax or modified wax constituents, such as n-butyl stearate or ethyl decanoate.

Exemplary fillers include, but are not limited to, Calcium carbonate, Talc, Silica, Glass fibers, Carbon black, Barium sulfate, Kaolin, Mica, Wollastonite, Alumina, Titanium dioxide, Cellulose, Wood flour, Fly ash, and Graphite.

Exemplary water or solvent chemically swellable particulate additives include, but are not limited to, poly(sodium acrylate) molecular weight ranging from 100 to 5 million daltons, more particularly 100 to 1 million daltons, more particularly 1000 to 1 million daltons, with particle sizes ranging from 1 micron to 1000 microns, more particularly 20 microns to 800 microns, more particularly 20 to 300 microns, in concentrations ranging from 0.01 to 95 wt,%, more particularly 0.1 to 75 wt %, more particularly 1 to 60 wt %, more particularly 2 to 55 wt %.

In some embodiments, particulate additives may decrease diffusion of water or other solvents into the adhesive (e.g., medical adhesive) composition and may be hydrophobic constituents such as steric acid, hydrophobic fumed silica or polyethylene waxes molecular weight ranging from 100 to 5 million daltons, more particularly 100 to 1 million daltons, more particularly 200 to 1 million daltons, with particle sizes ranging from 1 micron to 1000 microns, more particularly 20 microns to 800 microns, more particularly 20 to 300 microns, in concentrations ranging from 0.01 to 95 wt,%, more particularly 0.1 to 75 wt %, more particularly 1 to 60 wt %, more particularly 2 to 55 wt %.

In some embodiments, particulate additives may create physical sites for increased or reduced adhesion to skin and may be stimuli-responsive in nature. Constituents that create physical sites for enhanced or reduced adhesion to skin may be ceramic additives such as fumed silica, zinc oxide or titanium dioxide or may be polymers that have molecular weight ranging from 100 to 5 million daltons, more particularly 100 to 1 million daltons, more particularly 200 to 1 million daltons, with particle sizes ranging from 1 micron to 1000 microns, more particularly 20 microns to 800 microns, more particularly 20 to 300 microns, in concentrations ranging from 0.01 to 95 wt,%, more particularly 0.1 to 75 wt %, more particularly 1 to 60 wt %, more particularly 2 to 55 wt %.

Additives used herein may include nucleating agents for stimuli-responsive adhesives undergo change in adhesive behavior upon crystallization by cooling. The addition of nucleating agents such as nano-scale fumed silica and polyethylene waxes could be used to tune crystallization temperature of adhesives that exhibit crystalline transitions such as poly(octadecyl methacrylate). Nucleation-inducing additives include additives ranging in particle size from 1 nm to 1000 microns, more particularly 10 nm to 500 microns, more particularly 10 nm to 250 microns. Nucleation-inducing additives may be blended with adhesive in solution blending, high-shear mixing or other blending technique or may be generated in situ during adhesive preparation through techniques that include precipitation or phase separation. For example, stearic acid could be mixed with adhesive solutions under high shear conditions to form nanophases that remain dispersed in adhesive blends such as poly(stearyl) methacrylate.

In some embodiments, additives may serve as crack propagating agents to facilitate adhesive failure upon removal via mechanical peeling.

In some embodiments, the one or more additives are present in the adhesive (e.g., medical adhesive) composition in an amount from 0 to 60 wt %, more particularly from 0.1 wt % to 50 wt %, from 0.1 wt % to 40 wt %, from 0.1 wt % to 30 wt %, from 0.1 wt % to 20 wt %, from 0.1 wt % to 10 wt %, from 0.1 wt % to 5 wt %, from 0.1 wt % to 2.5 wt %, from 0.1 wt % to 1 wt %.

In some embodiments, the one or more additives is present in an amount of about 0.1 wt %, about 0.2 wt %, about 0.3 wt %, about 0.4 wt %, about 0.5 wt %, about 0.6 wt %, about 0.7 wt %, about 0.8 wt %, or about 0.9 wt % or more of the adhesive (e.g., medical adhesive) composition.

In some embodiments, the one or more additives is present in an amount of about 0.01 wt %, about 0.02 wt %, about 0.03 wt %, about 0.04 wt %, about 0.05 wt %, about 0.06 wt %, about 0.07 wt %, about 0.08 wt %, or about 0.09 wt % or more of the adhesive (e.g., medical adhesive) composition.

### 4.2.3. Method of Making the Stimuli-Responsive Polymer

In one aspect, the present disclosure provides a method of preparing a stimuli-responsive polymer described herein comprising curing one or more monomers and a first amount of one or more polyfunctional crosslinkers to form a prepolymer, and post-curing the prepolymer, thereby providing the stimuli-responsive polymer.

In some embodiments, the weight ratio of the one or more monomers to the one or more polyfunctional crosslinkers is from 98:2 to 99.9:0.1, e.g., from 98.5:1.5 to 99.9:0.1; from 99:1 to 99.9:0.1, from 99.1:0.9 to 99.9:0.1, from 99.2:0.8 to 99.9:0.1; from 99.3:0.7 to 99.9:0.1, from 99.4:0.6 to 99.9:0.1, from 99.5:0.5 to 99.9:0.1, from 99.6:0.4 to 99.9:0.1, from 99.7:0.3 to 99.9:0.1, from 99.8:0.2 to 99.9:0.1

In some embodiments, the curing is a photopolymerization utilizing photoinitiators. Exemplary photoinitiators include, but are not limited to, included 2,2-dimethoxy-2-phenylacetophenone (DMPA), Eosin y, diphenyl(2,4,6-trimethylbenzoyl)phosphine oxide (TPO), lithium phenyl-2,4,6-trimethylbenzoylphosphinate (LAP), and biocompatible photoinitiators.

In some embodiments, curing is done neat, i.e., in the absence of solvent.

In some embodiments, the post-curing comprises subjecting the prepolymer to an elevated temperature relative to the curing, e.g., at least 50 °C, at least 100 °C, at least 150 °C, or at least 200 °C.

In some embodiments, the method further comprises pausing curing prior to completion, adding a second amount of one or more polyfunctional crosslinkers, and restarting curing.

In some embodiments, polymers with C6 to C18 side chains or dangling chain ends are prepared from acrylate, methacrylate, alcohol, carboxylic acid, electron rich alkene, electron poor alkene, epoxy, amine, ROMP, Diels-Alder, Lactone, Lactam, Peptide, Peptoid, acrylamide, methacrylamide, thiol, vinyl and allyl monomers. In some embodiments, light crosslinking such as the netpoint concentration afforded by a range including but not limited to 0.4 wt %, 0.6 wt %, 0.75 wt %, 0.95 wt % trimethylolpropane triacrylate in a poly(lauryl) or poly(stearyl methacrlate polymer (including but not limited to 99.6 wt %, 99.4 wt %, 99.25 wt %, 99.05 wt %) lauryl or stearyl methacrylate) affords a shear rate responsive polymer that is tacky to human skin and that subsequently can be removed from human skin with minimal pain, and residual adhesive that remains on human skin can be removed by light rubbing with minimal pain.

In some embodiments, these long side chain/low crosslink density polymers can be prepared by any monomers, crosslinkers or other constituents described herein by any reaction process described herein. For example, linear or branched poly(ethyeneimine, PEI) can be modified on side chain and chain ends using laurl or octadecyl acrylate inder base catalyzed conditions via Michael addition or lauryl or octacecyl isocyanate using isocyanate/amine reaction. In another embodiment, off or on stoichiometric thiolenes can be prepared using combinations of mono, di, tri and tetrafunctional thiol and alkene monomers with monofunctional constituent such as lauryl mercaptopropionate or dodecyl vinyl ether such that monofunctional constituent comprises 0.1, 0.2, 0.3, 0.4 or 0.5 or more mole % of overall thiolene constituency. In another representative embodiment, an alternating copolymer comprising maleimide or n-butyl maleimide and docecyl vinyl ether is prepared using radical alternating polymerization and can be lightly crosslinked by dodecyl vinyl ether with less than wt % or a polymethacrylate or acrylate to form heterogeneous crosslinked networks with low crosslink density and high C12 side chain wt % (60, 70, 80, 90 or more wt % alkyl side chain C6 or greater with C12-C18 preferred). In another embodiment, octadecyl amine is polymerize red with a Michael addition co-monomer such as ethylene glycol diacrylate or hexanediol diacrylate under base catalyzed conditions. In one embodiment, the resulting poly(beta aminoester) is prepared with 1:25:1.0 C═C:NH2 (double NH2 reaction with acrylate) reaction such that acrylate end capped groups result, and acrylate end-capped poly(betamino ester) reaction products can be photocured using UV light, and crosslink density can be reduce by the addition of monofunctional acrylates or methacrylates such as stearyl or lauryl methacrylate or acrylate or by adding thiol chain transfer or capping agents such as PETMP or IOMP or EGBMP or 1,10-decanediothiol or PETMP. In another embodiment, a similar Michael addition synthetic process can be used for thiol/acrylate Michael addition reaction products, with excess acrylate preferred.

In another embodiment, the adhesive is a linear or branched crosslinked polymer network wherein the crosslinked polymer network contains 10 or more ester to thiolester linkages which hydrolyze in the presence of an added base or a compound containing a thiol, or a compound containing an amino wherein hydrolysis results in dissolution of the adhesive and delamination from penile skin.

In another embodiment, adhesive layer may be a liner or branched polymer comprising the reaction product by free radical addition polymerization of mono-, di-try-tetra-, penta, and hexa-functional thiol-ene constituents including triallyl isocyanurate pentaerythritol tetrakis (3-mercaptopropionate) or any of the thiol-ene monomeric constituents disclosed herein. One embodiment, thiol-ene adhesive may exhibit stimuli-responsive adhesive behavior upon cooling below its glass transition. In another embodiment, thiol-ene adhesive layer may exhibit chemically-responsive adhesive behavior such as oxidation of thioether linkages by common oxidizing agents such as hydrogen peroxide to form reversibly clearable disulfide linkages.

### 4.3. Articles

In one aspect, the present disclosure provides articles comprising the adhesive (e.g., medical adhesive) composition described herein adhered to a substrate. The articles described herein thereby have an adhesive surface suitable for adhering to, e.g., bodily surfaces or target sites. In certain embodiments, the surface is selected from glass, ceramic, composite, plastic/polymeric, metal, or any other common industrial surface.

In some embodiments, the adhesive (e.g., medical adhesive) composition is adhered to a portion of the substrate. In some embodiments, the adhesive (e.g., medical adhesive composition is continuous or discontinuous with the substrate. In some embodiments, the adhesive (e.g., medical adhesive) is coextensive with the substrate, i.e., adhered to the entire substrate.

In some embodiments, the substrate is a backing layer or release liner.

Exemplary backing layers include, but are not limited to, non-woven fabrics, woven fabrics, films (e.g., sheets), paper, composite materials obtained by laminating a film on a non-woven fabric or a woven fabric, or a combination thereof.

Non-woven fabric may include polyolefin resins such as polyethylene and polypropylene; polyester resins such as polyethylene terephthalate, polybutylene terephthalate and polyethylene naphthalate; rayon, polyamide, poly(ester ether), polyurethane, polyacrylic resins, polyvinyl alcohol, styrene-isoprene-styrene copolymers, and styrene-ethylene-propylene-styrene copolymers; and combinations thereof. Fabrics may include cotton, rayon, polyacrylic resins, polyester resins, polyvinyl alcohol, and combinations thereof. Films may include polyolefin resins such as polyethylene and polypropylene; polyacrylic resins such as polymethyl methacrylate and polyethyl methacrylate; polyester resins such as polyethylene terephthalate, polybutylene terephthalate and polyethylene naphthalate; and besides cellophane, polyvinyl alcohol, ethylene-vinyl alcohol copolymers, polyvinyl chloride, polystyrene, polyurethane, polyacrylonitrile, fluororesins, styrene-isoprene-styrene copolymers, styrene-butadiene rubber, polybutadiene, ethylene-vinyl acetate copolymers, polyamide, and polysulfone; and combinations thereof. Papers may include impregnated paper, coated paper, wood free paper, Kraft paper, Japanese paper, glassine paper, synthetic paper, and combinations thereof. In certain embodiments, the substrate is selected from backing layer selected from a polyurethane, poly(vinyl chloride), and latex.

In some embodiments, the substrate is elastomeric. In some embodiments, the substrate is rigid.

In some embodiments, the article has a planar or curved geometry selected from a square, circle, oval, hemisphere, rectangle, polygon, or curvilinear polygon.

In some embodiments, the article has a circle geometry. In some embodiments, the radius of the circle is at least about 0.5 cm, e.g., about 1.0 cm, about 2.0 cm, about 3.0 cm, or about 5.0 cm. In some embodiments, the radius of the circle is about 0.5 cm, about 1.0 cm, about 2.0 cm, about 3.0 cm, about 5.0 cm or about 10.0 cm or greater.

In some embodiments, the article has a rectangle geometry. In some embodiments, the rectangle has a length from 0.5 cm to 5 cm and a width from 0.5 cm to 5 cm. In some embodiments, the rectangle has a length from 0.5 cm to 4 cm, from 0.5 cm to 3 cm, from 0.5 cm to 2 cm, from 0.5 cm to 1 cm, from 1 cm to 5 cm, from 1 cm to 4 cm, from 1 cm to 3 cm, from 1 cm to 2 cm, from 2 cm to 5 cm, from 2 cm to 4 cm, from 2 cm to 3 cm, from 3 cm to 5 cm, or from 4 cm to 5. In some embodiments, the rectangle has a width from 0.5 cm to 4 cm, from 0.5 cm to 3 cm, from 0.5 cm to 2 cm, from 0.5 cm to 1 cm, from 1 cm to 5 cm, from 1 cm to 4 cm, from 1 cm to 3 cm, from 1 cm to 2 cm, from 2 cm to 5 cm, from 2 cm to 4 cm, from 2 cm to 3 cm, from 3 cm to 5 cm, or from 4 cm to 5.

In some embodiments, the article has an oval geometry. In some embodiments, the oval has a primary radius of about 0.5 cm, about 1.0 cm, about 2.0 cm, about 3.0 cm, about 5.0 cm or about 10.0 cm and a separate secondary radius of about 0.5 cm, about 1.0 cm, about 2.0 cm, about 3.0 cm, about 5.0 cm or about 10.0 cm or greater.

The primary dimensions of the article may vary. In one embodiment, the article has a width of between about 0.5 and about 50 cm, about 6 and about 40 cm, about 8 and about 30 cm, or about 10 and about 20 cm. In one embodiment, the length is between about 5 and about 250 cm, more particularly about 10 and about 225 cm, about 100 and about 200 cm, more particularly about 150 and about 175 cm.

In some embodiments, the article has primary dimensions of about 0.5×0.5 cm, about 1.0×1.0 cm, about 1.5×1.5 cm, about 2.5×2.5 cm, about 3.0×3.0 cm, or about 5.0×5.0 cm or any combination thereof in the case of a rectangular membrane.

In one embodiment, the article has the primary dimensions of about 6.0 cm×7.0 cm, about 10 cm×10 cm, about 10 cm×15 cm, about 10 cm×20 cm, about 10 cm×25 cm, about 10 cm×30 cm, about 10 cm×35 cm, about 7 cm×9 cm, about 6 cm×12 cm, about 9 cm×about 20 cm.

In one embodiment, the article has the primary dimensions of about 1.0 cm×3.0 cm, about 2 cm×7 cm, about 2.5 cm×8 cm, about 3.8 cm×10 cm, about 2 cm×7.5 cm, or about 3 cm×. 8 cm.

In one embodiment, the article has primary dimensions of about 2.4 in.×2.4 inc., 3 in.×3 in., 3 in.×8 in., 3 in. 16 in., 4 in.×. 4 in., 4 in.×5 in., 4 in.×6 in., 4 in.×8 in., 5 in.×. 3.5 in. 5 in.×4 in., 5 in.×6 in, 5 in.×8 in., 6 in.×6 in., 6 in.×8 in., 7 in.×. 5 in, 8 in.×8 in., 8 in.×10 in., 8 in.×20 in, 10 cm×. 20 cm, 10 cm×40 cm, 15×. 15 cm, 15×30 cm, or 25×. 25 cm.

In one embodiment, the article has a primary dimension of about 16 cm×16 cm, about 16 cm×18 cm, about 16 cm×20 cm, about 16 cm×24 cm, about 16 cm×26 cm, about 19 cm×21.5 cm.

In some embodiments, the substrate is a release liner, i.e., a layer which is removed from the medical adhesive composition following application to the bodily surface or target site. In some embodiments, the article further comprises a release liner adhered to the adhesive surface of the medical adhesive composition opposite the backing layer.

In some embodiments, the substrate remains in contact with the adhesive (e.g., medical adhesive) composition following application to the surface (e.g., bodily surface or target site). In some embodiments, the substrate provides mechanical strength.

The article may assume any suitable form. In some embodiments, the article may be flat, planar or three dimensional. In some embodiments, the article may be completely flexible, partially flexible, completely rigid or partially rigid.

The surface for application may be any suitable surface, including an internal or external surface.

The article may be any suitable article. In some embodiments, the article is a medical device or article. In some embodiments, the medical article is self-applied. In some embodiments, the medical article is applied by a healthcare professional. In other embodiments, the article is an industrial article or consumer article.

In some embodiments, the article is an adhesive patch. In some embodiments, the adhesive patch has two or more panels, e.g., left and right panels.

In some embodiments, the article may be formed to attach a first surface to a second surface (e.g., a joining device). In certain embodiments, the adhesive or article comprising the adhesive is a sealant, e.g., for an industrial purpose. In certain embodiments, the adhesive or article comprising the adhesive is not 3D printed.

In some embodiments, the article may be formed to attach a first tissue to a second tissue (e.g., a closure device).

In some embodiments, the article is a skin closure strip. In some embodiments, the article is a bacterial barrier, i.e., to prevent infection of a wound.

In some embodiments, the article is a wound dressing. Wound dressings are used to protect wounds from external factors (e.g., infectious agents) and to maintain a moist environment which is required for the healing process by managing the wound exudate. In some embodiments, the wound dressing comprises one or more adhesive layers, wherein at least one of the layers comprises or consists of the medical adhesive composition described herein (e.g., in the form of a membrane or film).

In some embodiments, the wound dressing comprises a medical adhesive composition described herein (e.g., a layer in the form of a membrane or film) and at least one (e.g., 1, 2, 3, 4) absorbent pad or layer for absorbing wound exudate.

In some embodiments, the wound dressing comprises a second adhesive layer overlying the first adhesive layer but on the opposite side of the absorbent layer.

In some embodiments, the absorbent pad or layer may comprise, for example, one or more of textiles, wovens, non-wovens, fabrics, bandages, gauze, or the like.

In some embodiments, the adhesive layer is a component of medical article (e.g., bandage, wound dressing or similar article), wherein the adhesive layer is thicker than the barrier layer of the bandage or wound dressing. In some embodiments, the adhesive layer is about 10%, about 20%, about 30%, about 40%, about 50%, about 60%, about 70%, about 80%, about 90%, about 100%, about 2×, about 3×, about 4×, about 5×, about 6×, about 7×, about 8×, about 9×, or about 10× or more thicker than the barrier layer.

The dressing may be used in connection with any suitable wound including, for example, chronic, subacute, acute, traumatic, and/or dehisced incisions, lacerations, punctures, avulsions, burns, ulcers or grafts. The wound may be a full thickness or partial thickness wound.

In some embodiments, the article is a dressing applied to a wound selected from a cut, abrasion, blister, burn, callous or other injury to the skin.

In some embodiments, the article is a surgical incision drape.

In some embodiments, the article may comprise a shielding layer on the opposite side of the target site (e.g., opposite of the wound side).

In some embodiments, the article may be formed to attach a tissue to a medical device. The medical device may be any suitable medical device. Representative, non-limiting medical devices includes tubes, catheters, IV lines, needles, and sensors.

A catheter is a soft, flexible tube (soft catheter) that can be attached to or inserted into the body (e.g., a body cavity, body vessel, or organ) temporarily or on a longer-term basis (e.g., an indwelling catheter). Representative, non-limiting catheters include transurethral or suprapubic catheters.

The sensor may be useful for determining any given variable. Representative non-limiting sensors include electric current sensor, magnetic or radio sensors, humidity sensors, fluid velocity or flow sensors, pressure sensors, thermal or temperature sensors, optical sensors, position sensor or the like. In some embodiments, the sensor described herein may be a vital sign sensor, such as a sensor for respiratory rate, heart rate, body temperature, or blood pressure. In some embodiments, the sensor is a disposable, electronic medical sensor. In some embodiments, the sensor is an analyte sensor. The analyte may be, for example, a naturally occurring substance, a non-naturally occurring substance, a metabolite or reaction product.

In some embodiments, the medical adhesive composition is used to adhere a medical device to a subject in need thereof such as an ostomy device (i.e., a temporary or permanent ostomy device) or wound therapy devices.

In some embodiments, the article is an eye cover, e.g., for use with an infant.

The article may be worn by a subject for a target time period, e.g., hours, days, weeks, months or longer, before being removed on-demand.

In some embodiments, when the adhesive surface of the article is applied to the skin surface of a subject, removal of the article by light peeling does not cause pain in the subject. In some embodiments, following adhesion of adhesive surface of the article to the skin, removal of the composition by light peeling causes minimal or no pain to the subject. Any suitable method can be utilized to determine the existence or absence of pain. There are several validated instruments for the measurement of pain. The instruments can be unidimensional, measuring only the intensity of pain, such as Wong-Baker Qualitative Pain Assessment (WBQPA), Numeric Pain Rating Scale (NRPS), visual analogue scale (VAS) and verbal rating scale. Multidimensional instruments measure the intensity, character and impact of pain, for example, the McGill Pain Questionnaire (MPQ) and Brief Pain Inventory (BPI).

In some embodiments, pain is measured using the WBQPA on a scale of 0 (no pain) to 10 (maximum pain). In some embodiments, the pain is less than 4 on the WBQPA scale when the article is removed by light peeling, e.g., less than 3, less than 2, less than 1 or 0. In some embodiments, a WBQPA score of 0 is reported upon removal of the article by light peeling. As discussed above, in some embodiments, light peeling corresponds to a user peel rate of the article of 25 mm/s or less, such as, for example, 10 mm/s or less, 5 mm/s or less, 1 mm/s or less, 0.5 mm/s or less, 0.3 mm/s or less, or 0.1 mm/s or less. In some embodiments, light peeling corresponds to a peel rate from 0.01 mm/s to 25 mm/s, such as, for example, from 0.01 mm/s to 10 mm/s, from 0.01 mm/s to 5 mm/s, from 0.01 mm/s to 1 mm/s, from 0.01 mms/to 0.5 mm/s, from 0.01 mm/s to 0.3 mm/s, from 0.01 mm/s to 0.1 mm/sec, from 0.1 mm/s to 25 mm/s, from 0.1 mm/s to 10 mm/s, from 0.1 mm/s to 5 mm/s, from 0.1 mm/s to 1 mm/s, from 0.1 mms/to 0.5 mm/s, from 0.1 mm/s to 0.3 mm/s, from 1 mm/s to 25 mm/s, from 1 mm/s to 10 mm/s, or from 1 mm/s to 5 mm/s.

In some embodiments, light peeling corresponds to a user peel rate of the article of 500 mm/min or less, such as, for example, 400 mm/min or less, 300 mm/min or less, 200 mm/min or less, 100 mm/min or less, or 50 mm/min or less, or 25 mm/min or less, or 10 mm/min or less, or 5 mm/min or less, or 1 mm/min or less, or 0.5 mm/min or less, or 0.3 mm/min or less, or 0.1 mm/min or less. In some embodiments, light peeling corresponds to a peel rate of 50 mm/min to 500 mm/min, such as, for example, from 50 mm/min to 400 mm/min, from 50 mm/min to 300 mm/min, from 50 mm/min to 200 mm/min. from 50 mm/min to 100 mm/min, from 100 mm/min to 500 mm/min, from 100 mm/min to 400 mm/min, from 100 mm/min to 300 mm/min, from 100 mm/min to 200 mm/min, from 200 mm/min to 500 mm/min, from 200 mm/min to 400 mm/min, from 200 mm/min to 300 mm/min, from 300 mm/min to 500 mm/min, from 300 mm/min to 400 mm/min, or from 400 mm/min to 500 mm/min.

In some embodiments, the site of adhesion exhibits limited or no irritation upon removal of the article. Various methods are known in research settings for quantitatively assessing skin damage. Certain of these models are based on measurement of baseline skin properties that change when skin is stressed. Measurable parameters such as skin hydration, trans-epidermal water low (TEWL), and irritation, among others, will provide information differentiating damaged skin from healthy skin. See, e.g., Bernatchez, S. et al, ADVANCES IN WOUND CARE, Vol. 2, No. 4 (2022), which is incorporated herein by reference. Reconstructed human epidermis models are also available that demonstrate reasonable similarities to the native human tissue in terms of morphology, lipid composition and biochemical markers. See, e.g., EpiSkin, SkinEthic and EpiDerm. Animal models of skin injury are also known in the art, including pigs and rodents. See, e.g., Summerfield, A. et al., Molecular Immunology, Vol. 66, Issue 1, July 2015, p. 14-21.

In some embodiments, wherein when the adhesive surface of the article is applied to the skin surface of a subject, removal of the article by light peeling results in less than 50 wt % of the adhesive remaining on the skin surface of the subject, e.g., less than 40 wt %, less than 30 wt %, less than 20 wt %, or less than 10 wt %.

Application of a stimulus to the adhered article causes the adhered article to become less adhesive or delaminate from the bodily surface, thereby enabling selective delamination (i.e., on-demand delamination). Reversible or irreversible adhesive behavior can be triggered by exposure to temperature changes (e.g., a decrease in temperature), physico-chemical changes (e.g., dissolution), light, ultrasound, ionic strength change, pH change, magnetic, electrical, or mechanical actions or forces, as well as other stimuli or any combination thereof.

In some embodiments, the adhered article becomes less adhesive or delaminates from the bodily surface in 0.1 s to 60 s in response to the stimulus, such as, for example, from 1 s to 60 s, from 1 s to 30 s, from 1 s to 15 s, from 1 s to 10 s, from 1 s to 5 s, from 2 s to 30 s, or from 1 s to 15 s.

In some embodiments, the stimulus is a mechanical action, e.g., shear rate. In some embodiments, the shear rate is induced by peeling or pulling on the adhered article at different rates or frequencies.

In some embodiments, the article is shear-rate responsive. In such embodiments, the article remains adhered upon application of higher shear rates and becomes less adhesive at lower shear rates. Exemplary higher shear rates include hard peeling or hard pulling. Exemplary lower shear rates include light peeling or light pulling. Higher and lower are taken with respect to a threshold about which a change in behavior is observed. In this manner, the article can be easily removed from the bodily following application of a stimulus to the article.

In some embodiments, the stimulus is a mechanical force. In some embodiments, the article is force responsive. In such embodiments, the article remains adhered upon application of higher forces and becomes less adhesive upon application of lower forces. Higher and lower are taken with respect to a threshold about which a change in behavior is observed.

In some embodiments, the applied force that results in delamination is, for example, from 0.01 to 0.1 N, from 0.1 to 1 N, from 1 to 10 N, from 10 to 100 N. or from 100 and 1000 N. In some embodiments, the applied force that results in delamination is, for example, from 1 to 10 Pa, from 10 to 100 Pa, from 0.1 to 1 kPa, from 1 to 10 kPa, from 10 to 100 kPa, or from 0.1 to 1 MPa.

In some embodiments, the stimulus is a physico-chemical change. An exemplary physico-chemical change is dissolution. In some embodiments, the stimulus is a change in dissolution of the stimuli-responsive polymer when contacted by a solvent. In some embodiments, the article adheres to the bodily surface in the absence of the solvent and is less adhesive or delaminates from the bodily surface when contacted by the solvent.

In some embodiments, the article exhibits chemically-responsive behavior and may be water or solvent soluble in nature and may be removed by pulling back edges and flowing water or solvent or rubbing a substrate impregnated with water or solvent until the article is removed by weakening of stimuli-responsive polymer or dissolution by water or solvent. Chemical delamination may also be achieved in this manner by pH-triggerable delamination by flowing or wiping in this manner with a substrate impregnated with a fluid with a pH suitable for skin contact that also achieves delamination of the composition or article from skin. Alternatively, chemical delamination may also be achieved by dissolution of the adhesive (e.g., medical adhesive) composition by flowing or wiping in this manner with a substrate impregnated with a fluid that achieves delamination from skin.

In some embodiments, the stimulus is temperature change. In some embodiments, the article adheres to a bodily surface at a temperature of 370 C and is less adhesive or delaminates from the bodily surface at a temperature of 25 °C or lower.

In some embodiments, the article exhibits thermally-responsive stimuli-responsive behavior that enables adhesion to a bodily surface at body temperature (about 37 °C) and also enables lowered adhesive force or delamination upon cooling below body temperature to about 25 °C, about 20 °C, about 15 °C, about 10 °C, about 5 °C, about 0 °C or lower. Cooling may be achieved, for example, by accelerated heat transfer by rubbing a wet substrate such as a water-impregnated towel or paper towel on the article or at the interface of the adhered substrate and medical adhesive composition while adhered to the bodily surface or by running water from a source such as a shower or a cloth or tissue wipe which contains a reagent which causes cooling (e.g., alcohol evaporation or dissolution of ammonium nitrate) to cool the article through a thermal transition such that delamination is achievable more easily than if the article were to be removed at body temperature.

### 4.4. Package

In one aspect, the present disclosure provides a package comprising an adhesive (e.g., medical adhesive) composition described herein.

In some embodiments, a package comprises an adhesive (e.g., medical adhesive) composition described herein. In some embodiments, a package comprises an article described herein.

In some embodiments, a package may be rigid, semi-rigid, flexible, or combination thereof in which the adhesive (e.g., medical adhesive) composition or article is placed in until used, wherein the package may or may not have additional uses beyond storage as described by herein. In some embodiments, the package may be 0.005 mm or thinner, 0.005 mm or greater, 0.01 mm or greater, 0.02 mm or greater, 0.03 mm or greater, 0.04 mm or greater, or 0.05 mm or greater. The package may be approximately rectangular, circular, elliptical polygonal, or curvilinear polygonal in shape.

In some embodiments, the package comprises a flexible wrapper comprising foil, plastic, plastic-lined paper, foil-lined paper, or a combination thereof.

In some embodiments, package is a blister-pack design. In some embodiments, the blister-pack design package comprises a semi-rigid plastic or paper or cardboard well covered by a thin metallic, paper or plastic film, which may be punctured by pressing on the bottom of the rigid well. Semi-rigid is defined to be a material and thickness that maintains its designed geometry, but may be deformed by exerting a force via of between 1 and 5 N (Newtons) or between 5 and 10 N or between 10 and 20 N, or between 20 and 100 N, or between 100 and 150 N, or between 150 and 200 N, or between 200 and 300 N. The deformation may be an elastic bending deformation, or a buckling deformation or a creasing deformation. The well may be cylindrical, conical, spherical, a body of revolution, polyhedral, irregular or non-symmetric in shape. The well may have a circular, rectangular, elliptical, polygonal or curvilinear polygonal cross section.

In some embodiments, the package is a well design. In some embodiments, the well design package comprises rigid plastic, paper, or cardboard construction. In some embodiments, a face or faces of the container are sealed via a removable plastic film or metal foil film. In some embodiments, a tab may protrude from the film, enabling it to be peeled away by finger-strength or another means of gripping the tab. In some embodiments, the sealing film may be punctured to expose the adhesive (e.g., medical adhesive) composition or article housed within.

In some embodiments, the package further comprises a delaminating composition suitable to induce delamination of the medical adhesive composition or article from a bodily surface when the composition or article has been adhered to a bodily surface.

In some embodiments, the delaminating composition is a wipe. In some embodiments, the wipe comprises a solvent that dissolves, denatures, or swells the stimuli-responsive polymer, thereby inducing delamination of the medical adhesive composition or article from the bodily surface when the composition or article has been adhered to the bodily surface and the wipe subsequently applied to the medical adhesive composition or article.

In some embodiments, the wipe comprises a volatile additive that cools the wipe upon evaporation, thereby inducing delamination of the medical adhesive composition or article from the bodily surface when the medical adhesive composition or article has been adhered to the bodily surface and the wipe subsequently applied to the medical adhesive composition or article.

### 4.5. Kits

In one aspect, the present disclosure provides a kit comprising an adhesive (e.g., medical adhesive) composition or article described herein and instructions for use.

In some embodiments, the adhesive (e.g., medical adhesive) composition is provided as a liquid. When provided as a liquid, the amount of the adhesive (e.g., medical adhesive) composition may vary. For example, the amount of the liquid adhesive composition may range from about 0.1 to about 10 mL. An applicator for application of the liquid adhesive composition to a treatment site may also be provided.

### 4.6. Methods of Use

In one aspect, the present disclosure provides methods of using the adhesive (e.g., medical adhesive compositions) and articles described herein.

The adhesive (e.g., medical adhesive) composition may be a preformed film or membrane. In alternative embodiments, the adhesive (e.g., medical adhesive) composition may be applied as a liquid or gel that is cured over the target site (e.g., bodily surface or wound, e.g., industrial surface).

In some embodiments, a method of closing a wound comprises applying a medical adhesive composition described herein to a wound having a first surface and a second surface separated by a wound cavity, wherein the composition is applied into the wound cavity; drawing the first surface and second surfaces of the wound together; and applying pressure sufficient to close the wound, whereby the composition adheres the first surface and second surface together. In some embodiments, pressure is applied with one or more fingers or the hand of the subject.

In some embodiments, a method of closing a wound comprising a first surface and a second surface separated by a wound cavity comprises drawing the first surface and second surface together to form a narrowed wound cavity; applying a medical adhesive composition described herein to the narrowed wound cavity; and applying pressure sufficient to close the wound, whereby the composition adheres the first surface and second surface together. In some embodiments, the first surface and the second surface are drawn together such that the first surface and the second surface are in contact and form a junction, and the method comprises applying the medical adhesive composition superficially to the junction.

In some embodiments, the first surface and the second surface are each independently selected from skin, mucosal tissue, connective tissue, epithelial tissue, muscle tissue, nervous tissue, cartilage, tendon, and bone. In some embodiments, the first and second surface are the same. In some embodiments, the first and second surface are different. In some embodiments, the first surface and the second surface are skin. In some embodiments, the first surface and the second surface are mucosal. In some embodiments, the first surface is skin tissue, and the second surface is cartilage or bone.

In some embodiments, the wound is selected from a surgical wound, a laceration, an abrasion, a puncture, a traumatic wound, a diabetic wound, a pressure wound, a burn wound, a chronic wound, and a combination thereof. The wound may be relatively small (e.g., non-invasive surgical wound) or relatively large (e.g., traumatic wound). When the wound is relatively large, the method may involve administering a liquid form of the adhesive composition disclosed herein, e.g., to fill the cavity.

In some embodiments, a method of or stabilizing, protecting, and/or healing a wound in a subject in need thereof is provided, comprising applying a medical adhesive composition or article described herein to a wound, thereby stabilizing, protecting, and/or healing a wound.

In some embodiments, a method of preventing bacterial infection at a target site (e.g., a wound) in a subject in need thereof is provided, comprising adhering a medical adhesive composition or article disclosed herein to a target site (e.g., a wound) at risk for bacterial penetration or infection, thereby preventing a bacterial infection.

In some embodiments, a method of preventing fluid ingress or egress from a target site (e.g., a wound) in a subject in need thereof is provided, comprising adhering a medical adhesive composition or article described herein to a target site (e.g., a wound), thereby preventing fluid ingress or egress from the target site.

In some embodiments, a method of reducing scarring at a target site (e.g., a wound) in a subject in need thereof is providing, comprising adhering a medical adhesive composition or article described herein to the target site (e.g., a wound), thereby reducing scarring compared to the absence of the medical adhesive composition or article. The reduction in scarring may vary. In one embodiment, the reduction in scarring is from about 1 to about 100%, or more particularly, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%. In certain embodiments, the conventional medical adhesive is a fibrin, cyanoacrylate, gelatin-resorcin formaldehyde/glutaraldehyde, and PEG-based glue known in the art.

In some embodiments, the medical adhesive composition or article provides adhesion (i.e., a sealant) at a target site that is not readily accessible or sutures or other closures are not easily implemented, e.g., pulmonary surgery. In some embodiments, the target site is soft tissue. In one embodiment, the soft tissue is lung, cardiovascular, skin, kidney, bladder, urethra, dura mater, liver, gastrointestinal tissues.

In some embodiments, a method of improving the efficacy of a skin grafting procedure in a subject in need thereof is provided, comprising adhering a medical adhesive composition or article described herein to a skin graft, thereby improving the efficacy of skin grafting compared to skin grafting in the absence of the medical adhesive composition or article. The improvement in efficacy may vary. In some embodiments, the improvement in efficacy is from about 1 to about 100%, or more particularly, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%. In certain embodiments, the conventional medical adhesive is a fibrin, cyanoacrylate, gelatin-resorcin formaldehyde/glutaraldehyde, and PEG-based glue known in the art.

In some embodiments, a method of applying an adhesive article to a subject comprises applying the adhesive surface of an article described herein to a wound or bodily surface of a subject. In some embodiments, the method further comprises removing the adhesive article after at least 1 second, e.g., 1 hour, 12 hours, 24 hours, 1 day, 1 or 1 week, wherein removal (a) causes minimal or no pain in the subject as measured by the WBQPA, e.g., a WBQPA score of less than 4, less than 3, less than 2, less than 1, or 0 or (b) result in medical adhesive-related skin injury (MARSI). In some embodiments, removal comprises application of a stimulus to the adhesive article comprising a stimuli-responsive polymer as described herein.

In some embodiments, the subject has a history of MARSI. In some embodiments, MARSI is selected from mechanical injury, irritation, inflammation, skin stripping, blister, skin tear, irritant contact dermatitis, allergic dermatitis, and maceration. In some embodiments, the subject has sensitive skin. In some embodiments, the subject is less than three months old or greater than 65 years old. In some embodiments, the subject has an underlying health condition. In some embodiments, the underlying health condition is selected from diabetes, renal insufficiency, immunosuppression, venous insufficiency, malnutrition, dehydration, and antibiotic use. In some embodiments, the bodily surface is selected from the skin of the head, neck, chest, upper limb, lower limb, hand, foot, abdomen, torso, back, buttocks, and genitals. In some embodiments, wherein the adhesive article is applied to a wound, and application of the adhesive article prevents bacterial infection of the wound. In some embodiments, wherein the adhesive article is applied to a wound, and application of the adhesive article forms a seal (i.e., prevents fluid ingress into or egress from the wound). In some embodiments, wherein the wound is selected from a surgical wound, a laceration, an abrasion, a puncture, a traumatic wound, a diabetic wound, a pressure wound, a burn wound, a chronic wound, or a combination thereof.

In some embodiments, removal of adhesive article causes no significant irritation, inflammation, or redness in the user.

In some embodiments, when the adhesive article is removed from the wound or bodily surface, 50 wt % or less of the adhesive remains on wound or bodily surface, such as, for example, 40 wt % or less, 30 wt % or less, 20 wt % or less, 10 wt % or less, or 5 wt % or less. In some embodiments, even if adhesive remains after removal, the properties of the adhesive are such that it is easily removed by light rubbing or rolling.

In some embodiments, the method of use comprising applying the adhesive (e.g., industrial adhesive, consumer adhesive) to a surface such as, for example, a glass surface, a plastic surface, a ceramic surface, or a metal surface. In some embodiments, the method prevents the leakage of fluid or gas, e.g., the adhesive serves as a sealant or gasket.

In some embodiments, the stimulus is mechanical action or force. In some embodiments, the mechanical action is shear rate. In some embodiments, the shear rate is induced by peeling, pulling, or rubbing at varying rates.

In some embodiments, the peeling is light peeling as described hereinabove.

In some embodiments, the stimulus is temperature change, and application of a stimulus comprises cooling the temperature of the adhesive article to 25 °C or lower

In some embodiments, the stimulus is physico-chemical change, and application of a stimulus comprises applying a wipe to the adhesive article, wherein the wipe comprises a solvent that dissolves, denatures, or swells the stimuli-responsive polymer, thereby inducing delamination from the wound or bodily surface. In some embodiments, the stimulus is physico-chemical change, and application of a stimulus comprises applying a wipe, wherein the wipe comprises a solvent that dissolves, denatures, or swells the medical adhesive composition, thereby inducing delamination of the adhesive article.

The subject for the methods described herein may be any suitable subject. In some embodiments, the subject is a human. In some embodiments, the subject is an infant or elderly human. In some embodiments, the subject is a neonate or premature infant. In some embodiments, the subject has an underlying medical condition (e.g., diabetes). In some embodiments, little or no residual is left on the surface (e.g., tissue) following delamination. In some embodiments, the subject is a surgical subject. In some embodiments, the subject is a non-invasive surgical subject.

In some embodiments, the subject may not have access to immediate medical treatment, due to circumstance (e.g., military scenarios) or location (e.g., rural location). In this circumstance, the medical adhesive compositions and articles disclosed herein may provide temporary closure (e.g., to limit bleeding) or provide temporary treatment of the wound.

The surface or target site may be any suitable surface or target site, e.g., a tissue or organ. The tissue may be connective tissue, epithelial tissue, muscle tissue, and nervous tissue. The tissue may be regular, normal, diseased or irregular. In some embodiments, the surface or target site is skin tissue. In some embodiments, the surface or target site is mucosal tissue, e.g., the oral mucosa, the nasal mucosa, the olfactory mucosa or the conjunctiva.

In some embodiments, the subject is at risk for medical adhesive-related injury or more particularly, medical adhesive-related skin injury (MARSI). The injury may be, for example, a mechanical injury (e.g., a tear or stripping injury), irritation or inflammation.

In some embodiments, the method disclosed herein reduces the risk of medical adhesive-related injury compared to a conventional medical adhesive by about 1 to about 100%, or more particularly, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95% or about 100%. In certain embodiments, the conventional medical adhesive is a fibrin, cyanoacrylate, gelatin-resorcin formaldehyde/glutaraldehyde, and PEG-based glue known in the art.

In some embodiments, the methods described herein reduce the risk of mechanical injuries to the skin (e.g. skin stripping or tearing) or mucosa by about compared to a conventional medical adhesive by about 1 to about 100%, or more particularly, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95% or about 100%. In certain embodiments, the conventional medical adhesive is a fibrin, cyanoacrylate, gelatin-resorcin formaldehyde/glutaraldehyde, and PEG-based glue known in the art.

In some embodiments, the methods described herein reduce the risk of irritation to the skin or mucosa about compared to a conventional medical adhesive by about 1 to about 100%, or more particularly, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95% or about 100%. In certain embodiments, the conventional medical adhesive is a fibrin, cyanoacrylate, gelatin-resorcin formaldehyde/glutaraldehyde, and PEG-based glue known in the art.

In some embodiments, the methods described herein reduce the risk of medical adhesive-related injury compared to a conventional medical adhesive used in a bandage by about 1 to about 100%, or more particularly, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95% or about 100%. In certain embodiments, the conventional medical adhesive is a fibrin, cyanoacrylate, gelatin-resorcin formaldehyde/glutaraldehyde, PEG-based glue, acrylate, polyacrylate, methacrylate, and polymethacrylate known in the art.

In some embodiments, the methods described herein reduce the risk of mechanical injuries to the skin (e.g. skin stripping or tearing) or mucosa compared to a conventional medical adhesive used in a bandage by about 1 to about 100%, or more particularly, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95% or about 100%. In certain embodiments, the conventional medical adhesive is a fibrin, cyanoacrylate, gelatin-resorcin formaldehyde/glutaraldehyde, PEG-based glue, acrylate, polyacrylate, methacrylate, and polymethacrylate known in the art.

In some embodiments, the methods described herein reduce the risk of irritation to the skin or mucosa compared to a conventional medical adhesive used in a bandage by about 1 to about 100%, or more particularly, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95% or about 100%. In certain embodiments, the conventional medical adhesive is a fibrin, cyanoacrylate, gelatin-resorcin formaldehyde/glutaraldehyde, PEG-based glue, acrylate, polyacrylate, methacrylate, and polymethacrylate known in the art.

Various methods are known in research settings for quantitatively assessing skin damage. Certain of these models are based on measurement of baseline skin properties that change when skin is stressed. Measurable parameters such as skin hydration, trans-epidermal water low (TEWL), and irritation, among others, will provide information differentiating damaged skin from healthy skin. See, e.g., Bernatchez, S. et al, ADVANCES IN WOUND CARE, Vol. 2, No. 4 (2022), which is incorporated herein by reference.

Reconstructed human epidermis models are also available that demonstrate reasonable similarities to the native human tissue in terms of morphology, lipid composition and biochemical markers. See, e.g., EpiSkin, SkinEthic and EpiDerm.

Animal models of skin injury are also known in the art, including pigs and rodents. See, e.g., Summerfield, A. et al., Molecular Immunology, Vol. 66, Issue 1, July 2015, p. 14-21.

In some embodiments, the methods described herein utilize the medical adhesive composition or article described herein and at least one therapeutic agent. In some embodiments, the medical adhesive composition or article contains at least one therapeutic agent selected from an anti-infective, a hemostatic agent, a pain-reducing agent or the like. In some embodiments, the at least one therapeutic agent is selected from the group consisting of anti-inflammatory analgesics; sedatives; local anesthetics; non-steroidal anti-inflammatory agents; antiallergic agents; anti-ulcer agents; antibiotics; antimicrobial agents; antiviral agents; antifungal agents; immunity inhibitors; naturally derived proteins or genetically engineered proteins; polysaccharides; glycoproteins or lipoproteins; oligonucleotides; polypeptide drugs; antibodies; antigens; chemotherapeutics; coagulant agents and hemostatic agents, such as prothrombin, thrombin, fibrinogen, fibrin, fibronectin, coagulation factors, tissue factors, collagen, gelatin, vasopressin, plasminogen activator inhibitors, platelet activators and synthetic peptides having hemostatic activity.

In some embodiments, an effective dose of agent (small molecule/peptide/nucleic acid) released from the wound closure material is at least about 0.1, about 0.2, about 0.3, about 0.4, about 0.5, about 0.6, about 0.7, about 0.8, about 0.9, about 1, about 2, about 3, about 4, about 5, about 6, about 7, about 8, about 9, or at least 10 or more mg over a suitable time period.

### 4.7. Methods of Manufacture

In one aspect, the present disclosure provides methods of preparing the adhesive articles described herein comprising adhering an adhesive (e.g., medical adhesive) composition to a substrate, wherein the adhesive composition comprises a stimuli-responsive polymer described herein.

In some embodiments, a method of preparing an article described herein comprises coating a prepolymer solution onto a substrate to provide a coated substrate, the prepolymer solution comprising one or more monomers and optionally one or more polyfunctional crosslinkers; curing the coated substrate; and post-curing the coated substrate thereby providing the article. The article may be a medical article or an industrial or consumer article.

In some embodiments, the curing is a photopolymerization.

In some embodiments, post-curing comprises subjecting the coated substrate to an elevated temperature relative to the curing, e.g., at least 50 °C, at least 100 °C, at least 150 °C, or at least 200 °C.

In some embodiments, the method further comprises pausing curing prior to completion, adding a second amount of one or more polyfunctional crosslinkers, and restarting curing.

In some embodiments, a method of manufacturing an article device described herein is provided comprising (i) providing a medical adhesive composition described herein and (ii) applying the adhesive to a substrate using one of the following methods: screen printing, pad printing, roll to roll coating, dip coating, thermoforming, extrusion, injection molding or other scaled methods of film manufacturing, thereby providing the adhesive article disclosed herein.

In some embodiments, the article comprises a medical adhesive composition (e.g., an adhesive layer) that is cured using photopolymerization. In some embodiments, curing is done using UV or visible light after application of the composition of monomers as a coating onto a r substrate. In some embodiments, the medical adhesive composition is fully or partially UV or visible light cured and then applied to a substrate.

In some embodiments, the medical adhesive composition is crosslinked after processing onto a release liner or barrier layer by latent reaction using the aforementioned crosslinking chemistries or others. For example, residual epoxide and alcohol or amine chemistries could be used to achieve crosslinking after dip coating an adhesive onto a release liner or barrier layer or applying via spray or in a roll-to-roll coating method.

In some embodiments, the article is self-healing, in that it can be prepared separately from a substrate, applied to a substrate in an additional step and optionally be made to undergo regenerative adhesive capability after removal from skin or other application surface. An example of a transfer process to apply a separately prepared adhesive layer to a substate is pad printing. Another example is the adhesive may be prepared separately from a substrate and extruded through an orifice or nozzle and transferred to a substrate. Another example is the adhesive may be prepared on a form or mould separately from the substrate, and the substrate transferred to the medical adhesive composition as a solid layer or a liquid deposited via dip coating, spray, brush, painting or rolling transfer.

In some embodiments, the article is subjected to one or more tests of its physical properties, including, but not limited to, tensile strength, tensile or overlap shear strength, peel adhesion strength, and/or impact strength, and provides satisfactory results.

In some embodiments, the article is subjected to one or more tests selected from: ASTM F2255-05 Standard Test Method for Strength Properties of Tissue Adhesives in Lap-Shear by Tension Loading; ASTM F2256-05 Standard Test Method for Strength Properties of Tissue Adhesives in T-Peel by Tension Loading; and ASTM F2258-05 Standard Test Method for Strength Properties of Tissue Adhesives in Tension and provides satisfy factory results.

### 4.8. Examples

Below are examples of specific embodiments for carrying out the present disclosure. The examples are offered for illustrative purposes only and are not intended to limit the scope of the present disclosure in any way. Efforts have been made to ensure accuracy with respect to numbers used (e.g., amounts, temperatures, etc.), but some experimental error and deviation should, of course, be allowed for.

While the examples below examples of the medical adhesive are for condoms and application to a particular skin type (penile skin), the medical adhesives disclosed herein are also suitable for applications other than condoms and for adhesion to non-penile skin.

### 4.8.1. Example 1

Poly(ethyl acrylate), average Mw~95 kDa by GPC, solids 18-22 wt % toluene solution was purchased from Sigma Aldrich and cast onto a polyethylene plastic substrate approximately 0.1 mm in thickness as measured by calipers. Toluene solvent was allowed to evaporate in a fume hood for approximately 72 hours. After solvent evaporation, a polymer film approximately 50 to 500 microns in thickness remained on the polyethylene substrate, with sections of the polymer film measured by calipers to be approximately 200 and 400 microns in thickness, respectively. This polymer film was sandwiched with a second polyethylene 0.3 mm layer and stored for 6 months at approximately 20 °C in approximately 45%-60% relative humidity. After 6 months, the poly(ethyl acrylate) film easily remained adhered to the 0.3 mm thick polyethylene film on which it was cast. To demonstrate thermally responsive delamination from human skin, the polyethylene/poly(ethyl acrylate) substrate was pressed onto a bare human arm, and adhesive behavior was demonstrated and recorded via video and shown to be strong enough to substantially limit facile removal of polyethylene/poly(ethyl acrylate) substrate from the human arm.

The adhered polyethylene/poly(ethyl acrylate) substrate attached to a human arm was run under room temperature water (approximately 20 °C) from a kitchen sink for approximately 5-10 seconds while also being subjected to light pulling/peeling hand forces, and delamination was achieved without pain or substantial applied hand force after 5-10 seconds with no observable adhesive residue on arm.

### 4.8.2. Example 2

Poly(ethyl acrylate), average Mw~95 kDa by GPC, solids 18-22 wt % toluene solution was purchased from Sigma Aldrich and cast onto commercially available 6″ by 6″ approximately 0.2 mm thick dental dam latex and polyurethane substrates. The dental dam was measured by calipers to be approximately 0.20 mm. Toluene solvent was allowed to evaporate in ambient air for 72 hours in approximately 45%-60% relative humidity. After solvent evaporation, a polymer film approximately 50 to 500 microns in thickness remained on the dental dam substrates, with sections of the polymer film measured by calipers to be approximately 200 and 400 microns in thickness, respectively.

Unlike the poly(ethyl acrylate) film that was stored for 6 months reported in Example 1, the poly(ethyl acrylate) film in Example 2 exhibited limited tack at room temperature (approximately 19 °C) when assessed for qualitative skin adhesion 72 h after casting onto latex and polyurethane dental dams.

To demonstrate thermally responsive delamination from human skin, a latex dental dam/poly(ethyl acrylate) substrate with a circular adhesive layer approximately 50 to 500 microns thick (with sections measured by calipers being approximately 200 and 400 microns thick, respectively) and approximately 4 cm in diameter as measured by calipers was pressed onto an erect human penis, and was heated using a hair dryer on a warm heat setting for approximately 5 seconds while pressing onto the penis head, and adhesive behavior was observed immediately after 5 second hair dryer exposure (oral fusion is an alternative technique to a hairdryer to heat a partially tacky adhesive in a hot melt process into a more ideal thermomechanical regime to achieve adhesion in one embodiment of the present disclosure). A rubber band was placed around the base of the tip of the penis to secure the non-adhesive section of the dental dam substrate and prevent any shear removal/stress concentrations from forming at non-adhered sites, and the erect penis was subjected to forces consistent with those observed during sexual intercourse using masturbatory techniques. Upon ejaculation, no semen was observed to leave the condom that was adhered to the head of the penis even after removal of the rubber band placed below the head of the penis. The adhered latex dental dam was then subjected to cool water (approximately 10-20 °C) in a common shower and lightly removed by peeling along the edges. Delamination with minimal or no pain in penile skin was observed while running cool water along the adhered dental dam/flowing water interface in approximately 10-20 seconds, and no adhesive residue was observed on penile skin or inside the urethra. Urination was easily achievable immediately after the removal of the adhered dental dam.

Example 2 was repeated and similar results were observed. When the methods of Example 2 were repeated, a paper towel was run across the base of the head of the penis, and no semen was observed on the paper towel. In example 2, a flaccid penis with adhered condom was dipped in ice water, and the condom was easily removed with no observable adhesive residue on the penis.

### 4.8.3. Example 3

Approximately 10 mL of Poly(ethyl acrylate), average Mw~95 kDa by GPC, solids 18-22 wt % toluene solution was added to a stainless steel mixing container. Approximately 1 mL of poly(butyl acrylate), average Mw 99 kDa by GPC, solids 25-30 wt %, was added to the same stainless steel mixing container and stirred using a polyethylene pipette for approximately 20 seconds. This Poly(ethyl acrylate)/poly(butyl acrylate mixture appeared homogeneous and optically transparent after stirring for approximately 20 seconds no visible signs of phase separation or precipitation. Both poly(ethyl acrylate) and poly(butyl acrylate) solutions were purchased from Sigma Aldrich and used as received. After mixing, approximately 3 mL of blended poly(ethyl acrylate)/poly(butyl acrylate) solutions were drawn into a 3 mL polyethylene pipette and dropped onto 6″×6″ latex dental dams in triplicate. Dental dams were approximately 200 microns in thickness as measured by calipers. Dental dam/adhesive solutions were allowed to evaporate for approximately 72 hours in ambient air conditions with approximately 40%-60% relative humidity at temperatures ranging from 15 C to 25 C. After solvent evaporation, a polymer blend film approximately 50 to 500 microns in thickness remained on the latex dental dam substrate, and sections of the adhesive/latex substrate were measuring using calipers such that adhesive the layer was measured to be approximately 200 and 400 microns thick, respectively. To demonstrate thermally responsive delamination from human skin, the latex/poly(ethyl acrylate)/poly(butyl acrylate substrate was pressed onto a bare human arm, and adhesive behavior was demonstrated to be strong enough to substantially limit facile removal of polyethylene/poly(ethyl acrylate)/poly(butyl acrylate substrate from the human arm. The adhered polyethylene/poly(ethyl acrylate)/poly(butyl acrylate) substrate attached to a human arm was run under cold water (approximately 5-10 C) from a refrigerator-chilled bottle while also being subjected to light pulling/peeling hand forces, and delamination was achieved without pain or substantial applied hand force after 2-5 seconds with no observable adhesive residue on arm and no observed pain in removal.

### 4.8.4. Example 4

A circular/ovular substrate was cut using common scissors from one of the triplicate latex dental dam/poly(ethyl acrylate)/poly(butyl acrylate) samples prepared using the methods of Example 3 such that an adhesive elastomeric substrate resulted for which covered approximately 100% of the latex surface after cutting. The resulting circular/ovular substrate had an approximate major diameter of 5.5 cm and a minor diameter of approximately 5 cm as measured by calipers. The resulting adhesive/latex substrate was then placed roughly symmetrically on the head of an erect human penis such that the urethra was centered on the adhesive substrate (“adhesive condom”) and the edges of the adhesive condom extended to approximately 50% to 75% surface area of the erect penile head, noting that the urethra was fully covered and that the base of the frenulum was exposed. When the adhesive condom was placed on the head of the erect penis, natural folding resulted that served as semen reservoirs.

After the adhesive condom was applied to the erect penis, the condom appeared securely fixed to the head of the penis. The erect penis with its head partially covered by the adhesive condom as described above was inserted into a human vagina after a light volume of common sexual lubricant was applied to the vagina, and sexual intercourse proceeded. Approximately every 15 seconds for several minutes the erect penis with applied adhesive condom was removed from the vagina and inspected, and the adhesive condom appeared to remain in place without delamination occurring from the head of the erect penis. After several minutes of sexual intercourse, ejaculation occurred, and the ejaculation began while the adhesive condom covered erect penis was in the vagina and concluded outside the vagina so that the ability of the adhesive condom to prevent external flow of semen during and ejaculation could be evaluated. No semen was observed by during or immediately following ejaculation. To remove the semen-containing adhesive condom, the surface and edges of the condom was exposed to cold water approximately 10 °C to 15 °C that was dispensed from a common shower head, and the condom was removed with minimal or no pain slowly, section by section, by hand by pulling back the edges of the condom in the presence of cold water for approximately 15-45 seconds. To the person with the penis in Example 4, ejaculation was easily achieved, and the sexual experience was notably different from that with a traditional condom that covers the head, frenulum and shaft of the penis, and for comparison, sexual intercourse was repeated with a lubricated latex condom and ejaculation was not achievable in the same sexual position as that in which the adhesive condom was used until the traditional latex condom was removed.

### 4.8.5. Example 5

Poly(n-dimethyl acrylamide) (“pDMAA”), Mw approximately 150,000 Da, was purchased from Scientific Polymer Products, Inc. Glycerol (>99%) was purchased from Sigma Aldrich, Inc. 50/50 pDMAA/glycerol blends by weight were prepared by dissolving 4.17 g of pDMAA, and 4.17 g glycerol in a solution mixture of (4.17 g acetone purchased from a local hardware store, technical grade and 2.50 g 190 proof ethanol). Homogeneous, generally clear solutions were observed upon mixing and heating pDMAA/glycerol/acetone/ethanol solutions to approximately 50 °C in sealed glass vials for 10 min and vortexing mixtures repeatedly (heat, vortex, repeat) until clear solutions were observed. pDMAA/glycerol solutions were then cast onto 6″×6″ latex dental dams and allowed to evaporate for approximately 48 to 96 hours at ambient temperatures in a chemical fume hood. After evaporation of acetone, ethanol and whatever water impurities were present in acetone/ethanol blends, the resulting pDMAA/glycerol films were approximately 400 microns thick and generally tacky to the touch at 20 °C and significantly more tacky/adhesive at 37 °C. A 5″×5″ 50/50 pDMAA/glycerol adhesive dental dam was applied to a human forearm arm and shown to exhibit significant adhesive force on video. This adhesive could be painlessly peeled from a human forearm while running tap water at room temperature over the human arm and was observed to be completely soluble in tap water.

### 4.8.6. Example 6

Poly(octadecyl methacrylate) (“pODMA”), Mw approximately 96,000 Da, was purchased from Scientific Polymer Products, Inc. in solution (approximately 20 wt %) in toluene. pODMA solutions were cast onto 6″×6″ latex dental dams and allowed to evaporate for approximately 48 to 96 hours at ambient temperatures in a chemical fume hood. After evaporation of toluene, the resulting p(ODMA) films were approximately 300 microns thick and not tacky to the touch at 20 °C (waxy in nature) and significantly tackier/adhesive at 37 °C. A 5″×5″ p(ODMA). The adhesive dental dam was applied to a human forearm arm and shown to exhibit significant adhesive force on video. This ODMA-containing substrate could be removed from human arm using ice water approximately 0 °C, and any residual ODMA remaining on human arm could be removed using olive oil or molten candle wax.

### 4.8.7. Example 7

The methods of example 7 were repeated to prepare a polymer blend on a latex dental dam comprising 1.0 parts poly(n-butyl acrylate), 6.50 parts poly(n-ethyl acrylate) and 3.0 parts poly(octadecyl methacrylate). Poly(n-butyl acrylate), Mw~99,000, and poly(ethyl acrylate), Mw~95,000 solutions in toluene were purchased from Sigma Aldrich and pODMA was purchased from Scientific Polymer Products as described in Example 6. In comparison to the 100% pODMA coated adhesive dental dams reported in Example 6, the three-component dental dams of Example 7 exhibited significantly less transfer of residual poly(ODMA) to human skin during delamination at 0 °C.

### 4.8.8. Example 8

Poly(ethylene) glycol (Mw 400 da) (PEG-400) and Poly(n-dimethyl acrylamide) (“pDMAA”), Mw approximately 150,000 Da, was purchased from Scientific Polymer Products, Inc. A 50/50 wt blend of PEG-400 and pDMAA was prepared in a 50 overall wt % solution of acetone and ethanol (solution was prepared using 4 g PEG-400, 4 g pDMAA, 4 g acetone (crude, from hardware store, likely water present) and 4 g 190 proof ethanol. A latex dental dam substrate taped down to a polypropylene Tupperware dish lid was placed in a vacuum oven at ambient pressure and at 65 °C with a constant air flow into a fume hood and allowed to equilibrate to 65 °C. The PEG-400/pDMAA 50/50 wt % solution was cast onto this 65 °C substrate for 24 h in vacuum oven under constant air flow conditions and ambient pressure. A resulting film was observed that exhibited strong adhesive behavior in the range of 45-65 °C (above the crystallization temperature of PEG-400) and no tack/adhesion at ambient temperature (below the crystallization temperature of PEG-400).

### 4.8.9. Example 9

A series of adhesives and barrier substrates was prepared by polymerization of monomeric species. Monomers including n-butyl acrylate, n-ethyl acrylate, n-octyl acrylate, n-hexyl acrylate, 2-hydroxyethyl methacrylate, isobornyl methacrylate, cetyl methacrylate, octadecyl methacrylate, lauryl methacrylate, acrylamide, n-isopropyl acrylamide, poly(ethylene glycol) diacrylate, Mn~750, trimethylolpropane triacrylate, ethoxylated trimethyololpropane triacrylate, pentaerithritol tetraacrylate and diurethane dimethacrylate, triethylene glycol divinyl ether, n-vinyl dodecyl ether (dodecyl vinyl ether), isooctyl 3-mercaptopropionate, triallyl isocyanurate, were purchased from Sigma Aldrich and/or Scientific Polymer.

Monomers were massed out using an analytical balance and added with 1.0 wt % DMPA photoinitiator until homogeneous solutions formed. These solutions were then injected between Rain-X coated 1.0 mm thick glass slides separated by 1.0 mm thick spacers and UV cured at 365 nm for 15 min in a UVP CL-1000 crosslinker, after which samples were post-cured at 90 °C under ambient conditions for approximately 10 hours. Samples, optically clear after post-curing at 90 °C, were then cooled to room temperature where they appeared optically clear to the naked eye and then placed in a freezer at −20 °C for 5 min, after which samples appeared to cloud or become opaque to varying extents. Samples could be removed from glass slides and handled. Initially room temperature samples with clouding behaved as nonstick waxy solids. Upon heating to body temperature samples became very adhesive to skin, more adhesive to polyurethane dental dams and less adhesive to latex dental dams. Samples that appeared optically clear and did not cloud after cooling behaved as gels with limited tack or adhesiveness to the polyurethane dental dams or latex dental dams.

In certain cases, samples with varying degrees of clouding exhibited the following properties: (A) waxy solids at room temperature, (B) adhesive to human skin after melting upon application to human skin and good simultaneous adhesion to latex and polyurethane condoms or dental dams, (C), frequency-responsive and/or shear responsive adhesive behavior such that melted wax adhesives maintained polyurethane and latex condom and dental dam barrier adhesion to human skin while barriers were “pulled” or “jerked” at higher forces or frequencies or shear rates such as those that would be expected to remove an adhesive barrier from human skin, while also exhibiting the ability to be removed from human skin with minimal or no pain by low frequency or low force or very soft or slow peeling. (D) For select samples, residual adhesive that remained on human skin after removal was melted and very difficult to remove, while in other cases adhesive could be very easily rubbed away with minimal pain or no pain in a very natural human manner. Videos were taken of observed behavior. Optically opaque samples that exhibited melting to human skin, including human forearm, bicep, shoulder and penis head skin and varying degrees of frequency, shear, or shear rate near-painless removal include UV cured poly(stearyl methacrylate samples) with varying crosslinkers that included poly(ethylene glycol) diacrylate, Mn 750, trimethylolpropane triacrylate, pentaerythrital tetraacrylate and ethoxylated trimethylolpropane triacrylate in crosslinker ranges that included 0 wt %, 0.2 wt %, 0. 4 wt %, 0.6 wt %, 0.75 wt %, 0.85 wt %, 0.95 wt %, 1.0 wt %, 2.0 wt %, 3.0 wt %, 5.0 wt %, 7.5 wt %, 10.0 wt %, 15.0 wt %, 20 wt %, 25 wt % and 30 wt % crosslinker prepared using 1.0 wt % DMPA photoinitiator.

Optically clear samples that exhibited strong adhesion to human skin, including human forearm, bicep, shoulder and penis head skin and varying degrees of frequency, shear, or shear rate near-,painless removal include UV cured poly(stearyl methacrylate samples) with varying crosslinkers that included poly(ethylene glycol) diacrylate, Mn 750, trimethylolpropane triacrylate, pentaerythrital tetraacrylate and ethoxylated trimethylolpropane triacrylate in crosslinker ranges that included 0 wt %, 0.2 wt %, 0. 4 wt %, 0.6 wt %, 0.75 wt %, 0.85 wt %, 0.95 wt %, 1.0 wt %, 2.0 wt %, 3.0 wt %, 5.0 wt %, 7.5 wt %, 10.0 wt %, 15.0 wt %, 20 wt %, 25 wt % and 30 wt % crosslinker prepared using 1.0 wt % DMPA photoinitiator.

Compositions that did not exhibit strong adhesion to human skin, including human forearm, bicep, shoulder and penis head skin, included poly(butyl acrylate) and poly(hexyl acrylate) crosslinked with 0.2, 0.4, 0.6, 1.0 and 2.0 or more wt % crosslinker that included poly(ethylene glycol) diacrylate, Mn 750, trimethylolpropane triacrylate, pentaerythrital tetraacrylate and ethoxylated trimethylolpropane triacrylate.

Compositions that did exhibit strong adhesion to human skin, including human forearm, bicep, shoulder and penis head skin included poly(lauryl methacrylate) and poly(octadecyl methacrylate) crosslinked with 0.4, 0.5, 0.6, 0.7 wt % trimethyolpropane triacrylate crosslinker and 1.0 wt % DMPA that were photopolymerized and post-cured at 90 C for 10 h or more. Higher concentrations of crosslinker such as 1.0 and 2.0 wt % TMPTA afforded skin-adhesive samples with less adhesive force.

### 4.8.10. Example 10

Adhesive samples were prepared by photopolymerizing a monomeric solution of 98.43 wt % lauryl methacrylate (LMA) and 0.59 wt % trimethylolpropane triacrylate (TMPTA), with 0.98 wt % 2,2-dimethoxy-2-phenylacetophenone (DMPA) photoinitiator. The monomers were massed out using an analytical balance and mixed until a homogenous solution was formed using a FlackTek SpeedMixer (DAC 330-100 PRO). Approximately 3 mL of solution was pipetted onto 25 mm disposable aluminum rheometer base plates (TA Instruments) and cured under a 365 nm UV lamp (UVP CL-1000 UV crosslinker) for 1 hour in an inert nitrogen atmosphere. The samples were then post-cured in at 120 °C for 1 hour at atmospheric pressure. The samples were then allowed to cool to ambient temperature.

A set of comparison samples were prepared using n-butyl acrylate (BA), TMPTA and DMPA. The comparison monomeric solutions had the following compositions: (a) 98.37 wt % BA, 0.55 wt % TMPTA, 1.08 wt % DMPA; (b) 98.27 wt % BA, 0.75 wt % TMPTA, 0.98 wt % DMPA; (c) 97.87 wt % BA, 1.16 wt % TMPTA, 0.98 wt % DMPA. Solutions (a-c) were injected between two Rain-X coated 1.0 mm thick glass slides which were separated by 1.0 mm of glass spacers. The samples were cured under a 365 nm UV light source (UVP CL-1000 UV crosslinker) for 15 minutes, and post-cured at 90 °C and atmospheric pressure for 12 hours, before cooling to ambient temperature. The samples were removed from the glass slides and placed on the surface of the 25 mm rheometer base plates.

Upon manual inspection, the adhesive samples and comparison samples were observed to be optically clear to the naked eye. All samples exhibited viscoelastic behavior without flow (a soft solid). It was observed that the adhesive sample exhibited strong tack to human skin (fingertip and inside of wrist), nitrile and glass, while the comparison samples exhibited elastomeric properties with little to no tack to human skin (fingertip or inside of wrist) and nitrile.

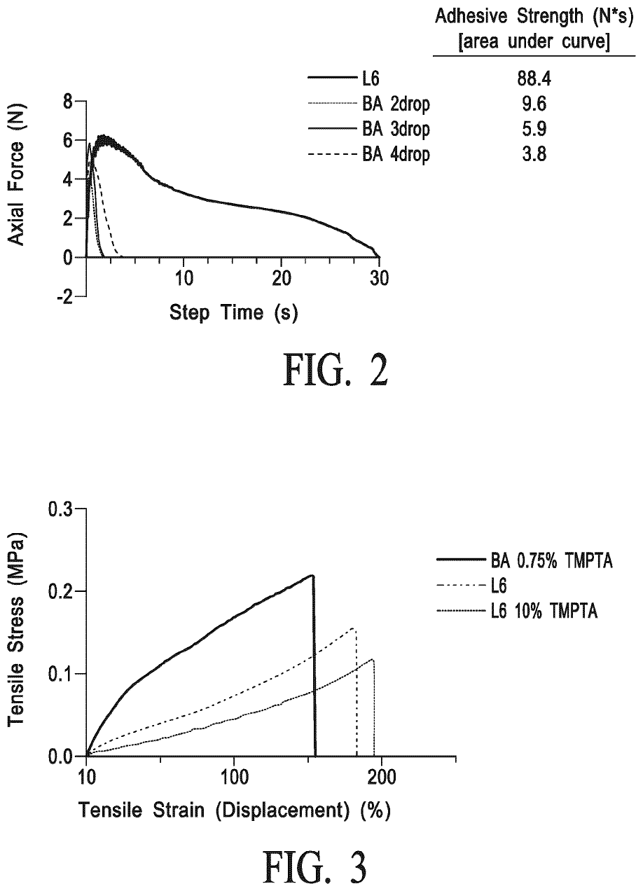

Tack strength was quantitatively measured using a TA Instruments Discovery HR-2 rheometer with a 8.00 mm aluminum tip and Peltier-cooled aluminum base plate, with measurements taken at a sample temperature of 25 °C. The sample was contacted and compressed for 60 seconds, after which the rheometer tip was withdrawn at a rate of 100 micrometers per second, and the axial force was measured. The maximum axial tack force measured was 6 N for the adhesive sample. The same process was repeated for the comparison samples, yielding peak tack forces of 5 N, 6 N, and 4 N for samples (a), (b) and (c) to aluminum, respectively. The adhesive strength, measured in Newton-seconds (Ns), is calculated by integrating the area under the force vs time measurements for each sample, yielding 88.4 Ns for the adhesive, and 9.6 Ns, 5.9 Ns and 3.8 Ns, for comparison samples (a), (b) and (c) (FIG. 2). The tack energy of the adhesive was also computed by multiplying the adhesive strength integral multiplied by the constant withdraw rate of 100 micrometers/sec, yielding a tack energy of 8.84×10−3 Joules (J) for the adhesive, and 0.96×10-3 J, 0.59×10-3 J and 0.38×10-3 J for the comparison samples (a), (b) and (c), respectively.

### 4.8.11. Example 11

A series of adhesive samples were prepared by photopolymerizing of monomeric solutions with the following composition: 98.43 wt % LMA and 0.59 wt % TMPTA with 0.98 wt % DMPA photoinitiator. The monomers were massed out using an analytical balance and mixed until a homogenous solution was formed using a FlackTek SpeedMixer (DAC 330-100 PRO).

A prepolymer solutions were formed by partially curing 90 g of the monomeric solution under a 365 nm UV light source (UVP CL-1000 UV crosslinker) for 14 minutes under a continuous-flow inert nitrogen atmosphere in a “pre-cure” step. This was repeated four individual times. Following the pre-cure, an additional mass of TMPTA was added to each of the four samples: (1) 0.00 wt % TMPTA (“L6”), (2) 0.06 wt % TMPTA (“L6 10% TMPTA”), (3) 0.12 wt % TMPTA, and (4) 0.18 wt % TMPTA. The solutions were again mixed until homogenous using a SpeedMixer.

Subsequently, 5 mL volumes of the prepolymer solutions were then pipetted onto silicone-impregnated paper affixed to a 1.0 mm thick glass slide and cured for a further 41 minutes under UV light in an inert nitrogen atmosphere. Upon removal from the crosslinker, the samples were each individually sandwiched between another piece of silicone-impregnated paper affixed to a 1.0 mm thick glass slide and compressed to a thickness of between 600-700 micrometers using spring clamps, and post-cured for 1 hour at 120 °C and atmospheric pressure, before cooling to ambient temperature. The samples were observed to be optically clear to the naked eye.

A tensile strain capacity assessment was performed using a universal testing machine (Instron 5944). Samples were measured with micrometers to be 600 microns thick, and were cut into 2 cm wide rectangular strips and clamed in the Instron fixtures, with a starting fixture separation of 5 cm. The sample was strained in tension at a rate of 300 mm/min until failure (FIG. 3) Sample (1) failed at 0.15 MPa stress at 182% strain. Sample (2) failed at 0.12 MPa stress and 217% strain. Sample (4) failed at 0.067 MPa stress and 120% strain.

An additional non-adhesive comparison sample was prepared from a monomeric solution comprising 98.27 wt % BA, 0.75 wt % TMPTA, 0.98 wt % DMPA; which was injected between two Rain-X coated 1.0 mm thick glass slides which were separated by 1.0 mm of glass spacers. The samples were cured under a 365 nm UV light source (UVP CL-1000 UV crosslinker) for 15 minutes, and post-cured at 90 °C and atmospheric pressure for 12 hours, before cooling to ambient temperature. This sample failed at 0.22 MPa stress and 152% strain.

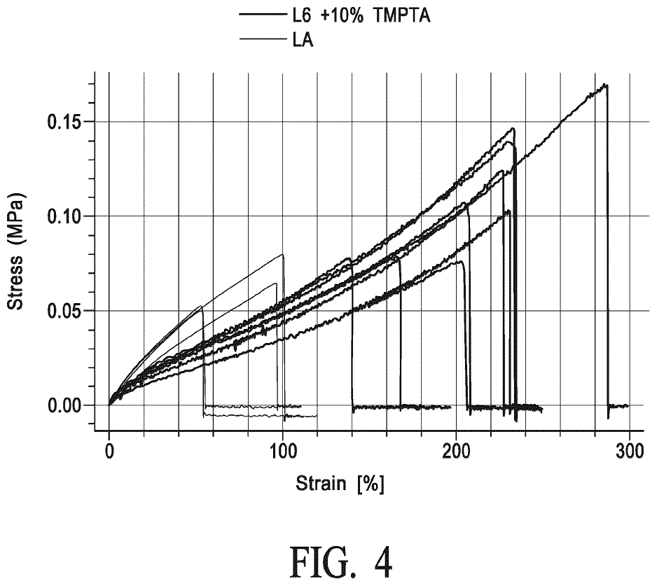

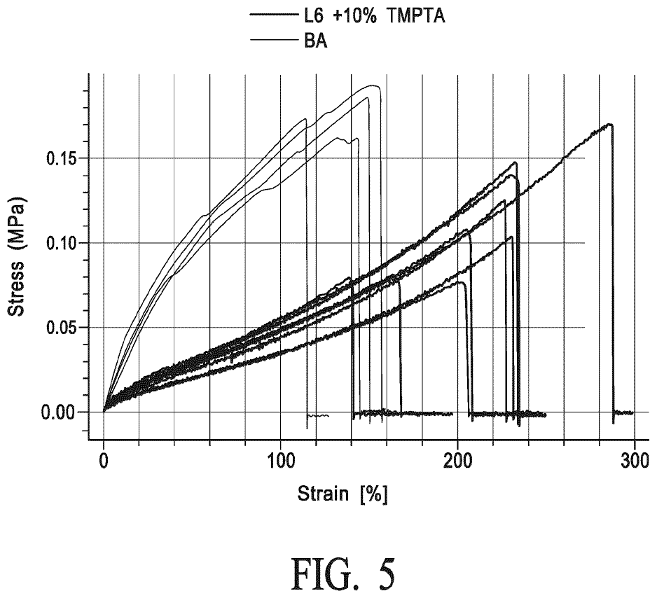

Additional strain capacity measurements were performed on L6 10% TMPTA samples, BA samples and LA samples. The samples thicknesses were measured, cut into 1.6 cm wide rectangular strips and clamed in the Instron fixtures, with a starting fixture separation of 3 cm. The sample was strained in tension at a rate of 300 mm/min until failure (FIG. 4 and FIG. 5). L6 10% TMPTA had a significantly higher strain capacity than the LA elastomer. The Young's modulus was computed from the slope of the stress-strain curve between 0% and 5% strain and found to be 1.33±0.47 kPa for L6 10% TPMTA, 1.33±0.40 kPA for LA and 3.15±1.13 kPA for BA.

Alien tape, a commercially available thick elastomeric nano-texture tape, was also tested in the same manner. It did not fail before the maximum extents of the Instron were reached.

### 4.8.12. Example 12

Adhesive samples were prepared by photopolymerizing a monomeric solution of 98.43 wt % lauryl methacrylate (LMA) and 0.59 wt % trimethylolpropane triacrylate (TMPTA), with 0.98 wt % 2,2-dimethoxy-2-phenylacetophenone (DMPA) photoinitiator. The monomers were massed out using an analytical balance and mixed until a homogenous solution was formed using a FlackTek SpeedMixer (DAC 330-100 PRO). Approximately 1.5 mL of solution was pipetted onto 75 by 50 mm glass slides, 1.0 mm in thickness, and cured under a 365 nm UV lamp (UVP CL-1000 UV crosslinker) for 1 hour in an inert nitrogen atmosphere. The samples were removed from the crosslinker and sandwiched between another piece of silicone paper affixed to a 1.0 mm thick glass slide, and compressed using spring clips, yielding a gap thickness of approximately 100 microns. The samples were then post-cured in at 120 °C for 1 hour at atmospheric pressure, and then left to cool to ambient temperature.

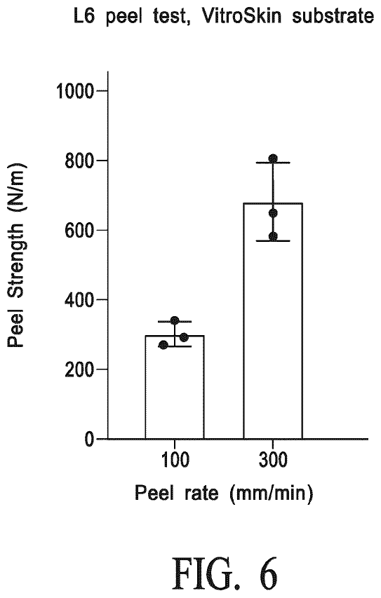

180-degree Peel tests were conducted using a universal testing machine (Instron 5944) and Vitro-Skin (Florida Suncare Testing Inc, IMS Division) as a human-skin substrate analog (FIG. 6). The A 5 cm wide rectangular strip of Vitro-Skin was pressed onto the adhesive with moderate finger pressure and left to settle for 1 minute prior to testing. The glass slide was affixed in a stationary position to the lower Instron fixture. The Vitro-Skin was affixed to the upper fixture in a standard 180-degree peel test configuration. The Vitro-Skin was peeled from the adhesive at 100 mm/min and repeated for three samples. The average peel strength (force per unit width) was measured to be approximately 300 N/m. The procedure was repeated for a peel rate of 300 mm/min, yielding an average of approximately 700 N/m, respectively, illustrating a shear-rate responsive behavior of the adhesive, that at very low peel rates, the peeling force is low, and at high peel rates, the adhesion force remains high.

Comparative analyses were carried for 3M Tegaderm, a commercially available medical adhesive. A 26 mm wide rectangular strip of Tegaderm was pressed onto a clean glass slide with a 500-gram mass for 1 minute prior to testing. The glass slide was affixed in a stationary position to the lower Instron fixture. The Tegaderm was affixed to the upper fixture in a standard 180-degree peel test configuration. The adhesive was again peeled from the glass slide at 100 mm/min, and 300 mm/sec, repeated 10 times for each peel rate. Average peel forces were approximately 98 N/m, 94 N/m, respectively, indicating no significant shear-rate dependence of the adhesive.

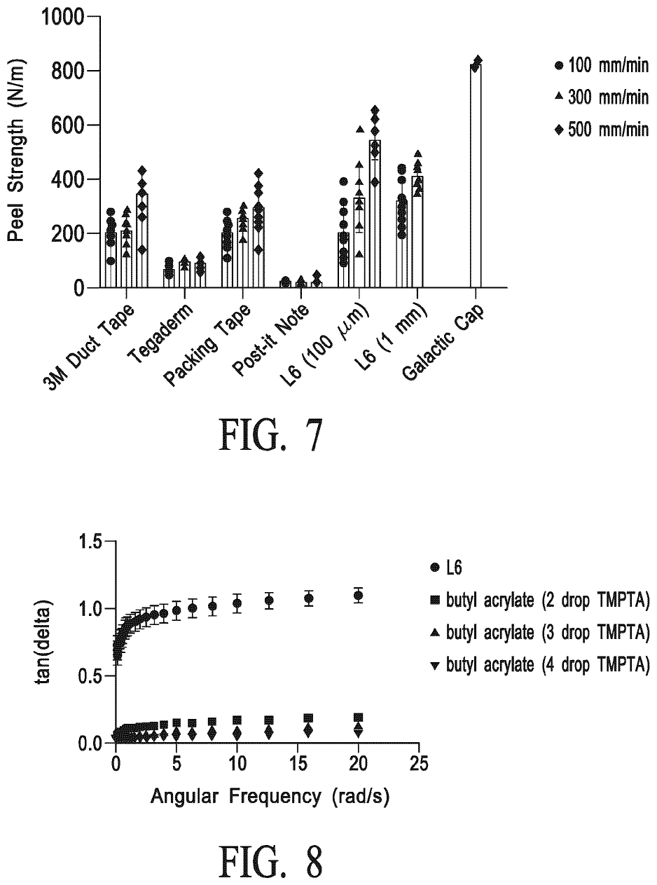

Additional comparisons were carried out on several other commercially available adhesives (FIG. 7). For a conventional pressure-sensitive adhesive, variations observed in the peel force required for removal are predominantly influenced by the pressure used to apply the adhesive to the target surface, and optionally time in contact with the target surface, in comparison to the speed or peel rate at which the adhesive is removed, which have no influence or a weak influence on the delamination behavior (e.g., peel strength).

In contrast, the stimuli-responsive adhesives described herein exhibit a different behavior (peel strength or perceived pain) during delamination at different peel rates that is predominantly influenced by the peel rate or shear rate of the adhesive as it is being removed (or another stimulus applied to the adhesive immediately before delamination). The delamination behavior is independent or weakly dependent on the pressure used to apply the adhesive to the target surface, and optionally the time in contact with the target surface. (These statements assume target surface, environmental variables, temperature, humidity, are all controlled to be equivalent).

### 4.8.13. Example 13

Adhesive samples were prepared by photopolymerizing a monomeric solution of 98.43 wt % lauryl methacrylate and 0.59 wt % trimethylolpropane triacrylate (TMPTA), with 0.98 wt % 2,2-dimethoxy-2-phenylacetophenone (DMPA) photoinitiator. The monomers were massed out using an analytical balance and mixed until a homogenous solution was formed using a FlackTek SpeedMixer (DAC 330-100 PRO). Approximately 3 mL of solution was pipetted onto 25 mm disposable aluminum rheometer base plates (TA Instruments) and cured under a 365 nm UV lamp (UVP CL-1000 UV crosslinker) for 1 hour in an inert nitrogen atmosphere. The samples were then post-cured in at 120 °C for 1 hour at atmospheric pressure. The samples were then allowed to cool to ambient temperature.

A set of comparison samples were prepared using n-butyl acrylate (BA), TMPTA and DMPA. The comparison monomeric solutions had the following compositions: (a) 98.37 wt % BA, 0.55 wt % TMPTA, 1.08 wt % DMPA; (b) 98.27 wt % BA, 0.75 wt % TMPTA, 0.98 wt % DMPA; (c) 97.87 wt % BA, 1.16 wt % TMPTA, 0.98 wt % DMPA. Solutions (a-c) were injected between two Rain-X coated 1.0 mm thick glass slides which were separated by 1.0 mm of glass spacers. The samples were cured under a 365 nm UV light source (UVP CL-1000 UV crosslinker) for 15 minutes, and post-cured at 90 °C and atmospheric pressure for 12 hours, before cooling to ambient temperature. The samples were removed from the glass slides and placed on the surface of the 25 mm rheometer base plates.

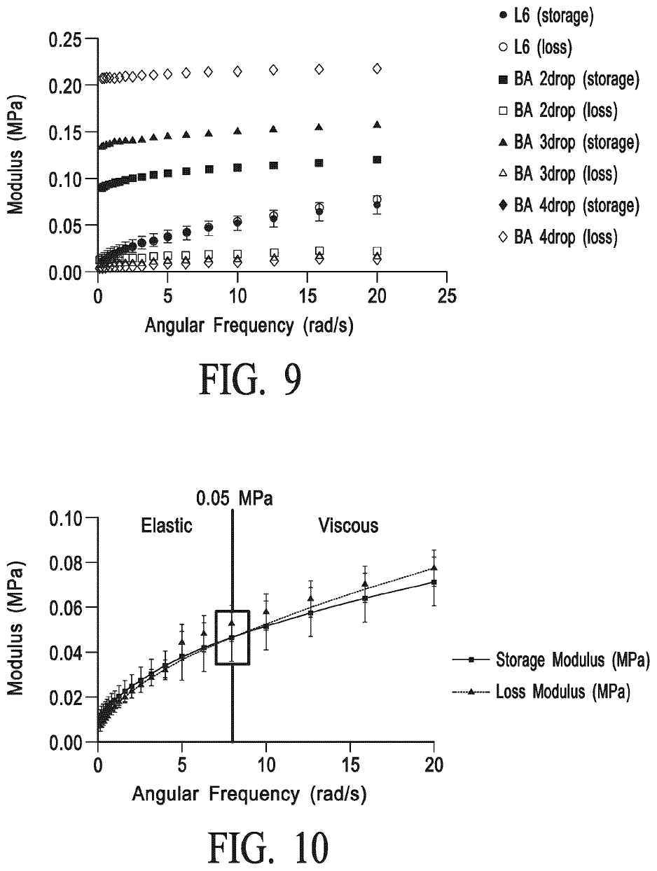

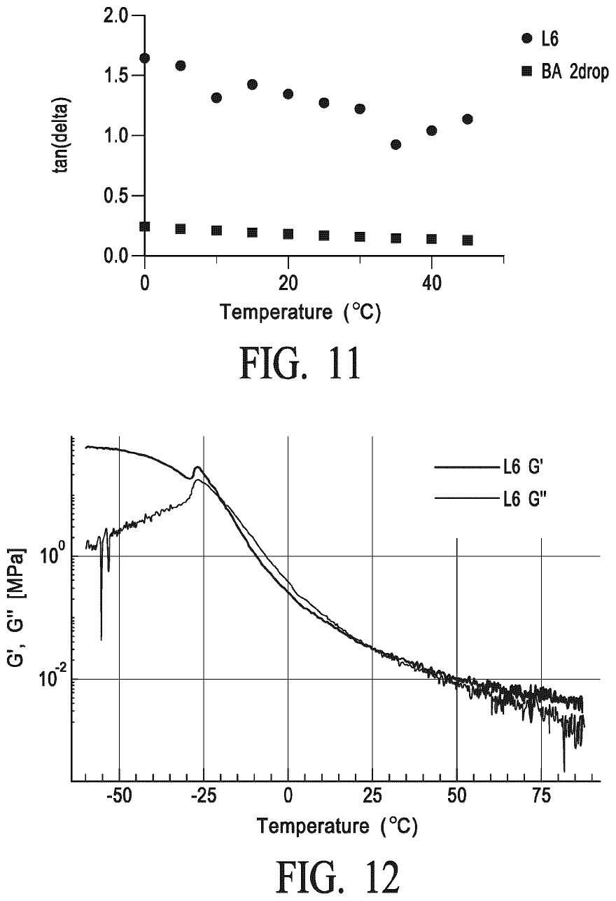

Oscillating temperature sweeps were performed on a TA Instruments Discovery HR-2 rheometer. A strain oscillation amplitude sweep was performed on the adhesive sample at 25 °C to determine the nominal oscillating strain amplitude. The storage and loss moduli curves were found to be constant, and a 1% strain was selected (FIG. 9 and FIG. 10). A logarithmic sweep in angular frequency from 1.0 to 100.0 rad/sec was performed with 5 points per decade, and over a temperature sweep of from 0 °C to 50 °C in 5 °C steps (FIG. 8 and FIG. 11). A high loss modulus for L6 was observed by a high tan(delta) value of between 0.5 at low frequencies to 0.9 at high frequencies. In contrast, the comparison samples (denoted by “butyl acrylate (2 drop TMPTA)” (b), “butyl acrylate (3 drop TMPTA)” (c) and “butyl acrylate (4 drop TMPTA) (d)) had a significantly lower loss modulus, near approximately 0.1 throughout the frequency range.

### 4.8.14. Example 14

Adhesive samples were prepared by photopolymerizing of monomeric solutions with the following composition: 98.43 wt % LMA and 0.59 wt % TMPTA with 0.98 wt % 2DMPA photoinitiator. The monomers were massed out using an analytical balance and mixed until a homogenous solution was formed using a FlackTek SpeedMixer (DAC 330-100 PRO).

A prepolymer solution was formed by partially curing 90 g of the monomeric solution under a 365 nm UV light source (UVP CL-1000 UV crosslinker) for 14 minutes under a continuous-flow inert nitrogen atmosphere in a “pre-cure” step. Subsequently, 5 mL of the prepolymer solution was pipetted onto silicone-impregnated paper affixed to a 1.0 mm thick glass slide and cured for a further 41 minutes under UV light in an inert nitrogen atmosphere. Upon removal from the crosslinker, the samples were sandwiched between another piece of silicone-impregnated paper affixed to a 1.0 mm thick glass slide and compressed to a thickness of between 600-700 micrometers using spring clamps, and post-cured for 1 hour at 120 °C and atmospheric pressure, before cooling to ambient temperature. The samples were observed to be optically clear to the naked eye.

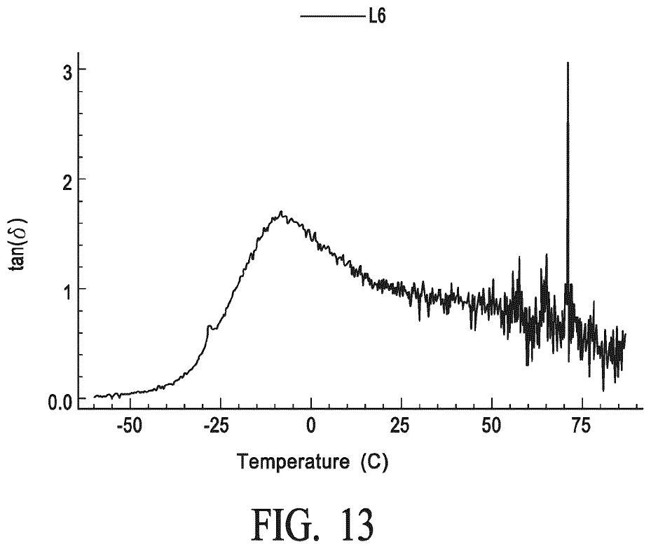

Dynamic Mechanical Analysis (DMA) was performed using a Mettler Toledo DMA 1-Star instrument. The samples were performed by carefully cutting them into discs measuring approximately 0.80 mm in thickness and 4.75 mm in diameter. The shear deformation mode was selected, with a displacement limit of 1.0 micrometers. A temperature sweep was carried out from −50 °C to 100 °C, with a gradual heating rate of 3 °C per minute. Additionally, the frequency of deformation was set at 2 Hz. The storage and loss moduli were measured (FIG. 12), as well as tan(δ) (FIG. 13).

### 4.8.15. Example 15

LMA, TMPTA and 1.0 wt % DMPA photoinitiator were mixed until a homogeneous solution formed and were then UV cured at 365 nm in polypropylene boxes (approximately 0.1-1.0 mm thick samples) under nitrogen for 45 min in a UVP CL-1000 crosslinker), after which samples were post-cured at 120 °C under ambient conditions for approximately 1 hour. Samples, optically clear after post-curing at 120 °C, were then cooled to room temperature where they appeared optically clear to the naked eye. Samples exhibited excellent tack to human skin and could be removed with minimal or no pain.

Samples were also prepared separately using a photoreactor. A magnetic stir plate was placed under a UVP CL-1000L crosslinker, and approximately 100 mL of homogeneous LMA, TMPTA and 1.0 wt % DMPA photoinitiator was stirred at approximately 180 RPM using a magnetic stir bar while being irradiated at 365 nm for approximately 15-20 minutes, after which viscosity increased to all more uniform adhesive film coatings to be prepared. Optionally additional TMPA (10%, 20% or 30% increase from original TMPTA composition) was added after initial 15 to 20 minute irradiation and mixed for 15 minutes using a FlackTek SpeedMixer (DAC 330-100 PRO) at 800 rpm to assure homogeneous mixing. Additional TMPTA was added to account for converted TMPTA used to build molecular weight and increase viscosity that might not be incorporated into network. Adhesive coatings ranging in thickness from 0.025 mm to 1.75 mm made from increased viscosity prepolymers were prepared from increased viscosity LMA/TMPTA/DMPA mixtures on top of silicone release liner paper under nitrogen and irradiation in UVP CL-1000L crosslinker at 365 nm for 45 min and thermal post cure at 120 °C for 1 hour. Release liner/adhesive coating layers could be sandwiched between an additional silicone release liner paper and stored for further use and process demonstrated is consistent with that suitable for use in roll-to-roll UV coating of adhesives on release liners.

The roll or sheets of adhesives may be fabricated into a condom device through a suitable transfer process to mate the adhesive to a latex, elastomeric or other polymeric barrier layer. Additionally, multiple layers of adhesive may be laminated together to form a composite adhesive layer with a greater thickness if desired using a suitable roll-to-roll or sheet laminating fabrication process.

### 4.8.16. Example 16

Samples were prepared of monomeric species and DMPA photoinitiator to form linear polymers. Three compositions were prepared: (a) 99.0 wt % lauryl methacrelate (LMA) and 1.0 wt % DMPA, (b) 99.0 wt % lauryl acrylate (LA) and 1.0 wt % DMPA, and (c) 99.0 wt % butyl acrylate (BA) and 1.0 wt % DMPA using analytic balances. Each solution was mixed using a FlackTek SpeedMixer (DAC 330-100 PRO) at 400 rpm until a homogenous solution was achieved. Approximately 6 mL of solution was pipetted into 1 inch by 3 inch compartments in a polypropylene tray, for a total of 12 compartments per solution. The solutions were UV cured (UVP CL-1000 crosslinker) under a nitrogen inert atmosphere for 1 hour, after which they were thermally post-cured for 1 hour at 120 C. After post-curing the samples cooled to ambient temperature and were observed to be optically clear.

Samples of 2 mg/mL solution of each cured polymer were prepared in HPLC-grade THF. The solutions were filtered through 0.22 micron PTFE filters and 0.5 microliters were injected on GPC with THF as eluent and polystyrene standards. A 1 mL/min flow rate and a light scattering detector were used. It was found that the BA sample had an average molecular weight of 1381218 g/mol and a polydispersity index (PDI) of 1.2. The LMA sample was found to have an average molecular weight of 27040 g/mol and a PDI of 2.4. The LA sample was found to have an average molecular weight of 759767 g/mol and a PDI of 3.2.

### 4.8.17. Example 17

Crosslinked adhesive samples were prepared by photopolymerizing a monomeric solution of 98.43 wt % lauryl methacrylate and 0.59 wt % trimethylolpropane triacrylate (TMPTA), with 0.98 wt % 2,2-dimethoxy-2-phenylacetophenone (DMPA) photoinitiator (L6). The monomers were massed out using an analytical balance and mixed until a homogenous solution was formed using a FlackTek SpeedMixer (DAC 330-100 PRO). Approximately 3 mL of solution was pipetted onto 25 mm disposable aluminum rheometer base plates (TA Instruments) and cured under a 365 nm UV lamp (UVP CL-1000 UV crosslinker) for 1 hour in an inert nitrogen atmosphere. The samples were then post-cured in at 120 °C for 1 hour at atmospheric pressure. The samples were then allowed to cool to ambient temperature.

Comparison samples comprised of linear polymers were prepared by photopolymerizing monomeric solutions of (a) 99.0 wt % lauryl methacrylate (LMA) and 1.0 wt % DMPA, and (b) 99.0 wt % butyl acrylate (BA) and 1.0 wt % DMPA using analytic balances. Each solution was mixed using a FlackTek SpeedMixer (DAC 330-100 PRO) at 400 rpm until a homogenous solution was achieved. Approximately 6 mL of solution was pipetted into 1 inch by 3 inch compartments in a polypropylene tray, for a total of 12 compartments per solution. The solutions were UV cured (UVP CL-1000 crosslinker) under a nitrogen inert atmosphere for 1 hour, after which they were thermally post-cured for 1 hour at 120 °C. After post-curing, the samples cooled to ambient temperature and were observed to be optically clear. Approximately 0.5 ml_, of the polymer samples were scooped onto 25 mm disposable aluminum rheometer base plates and allowed to settle for 30 minutes.

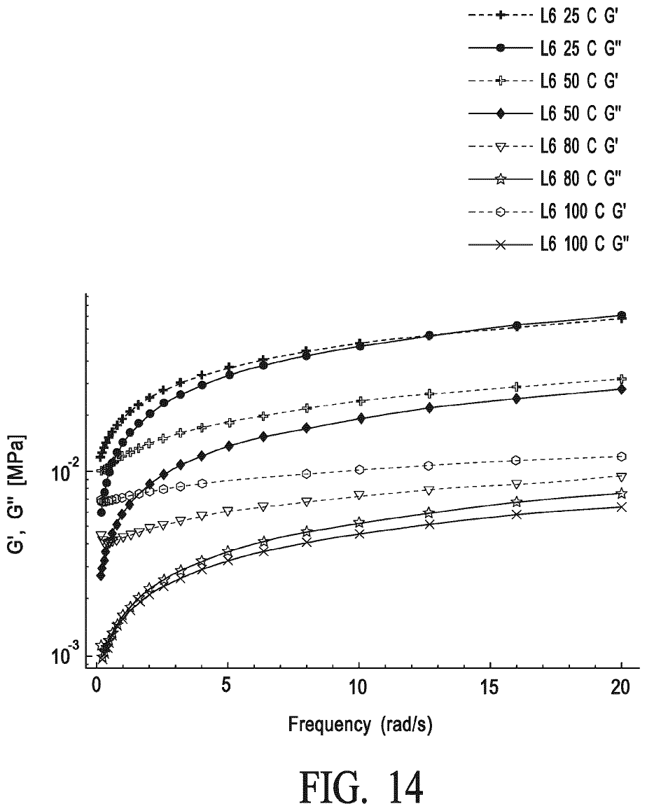

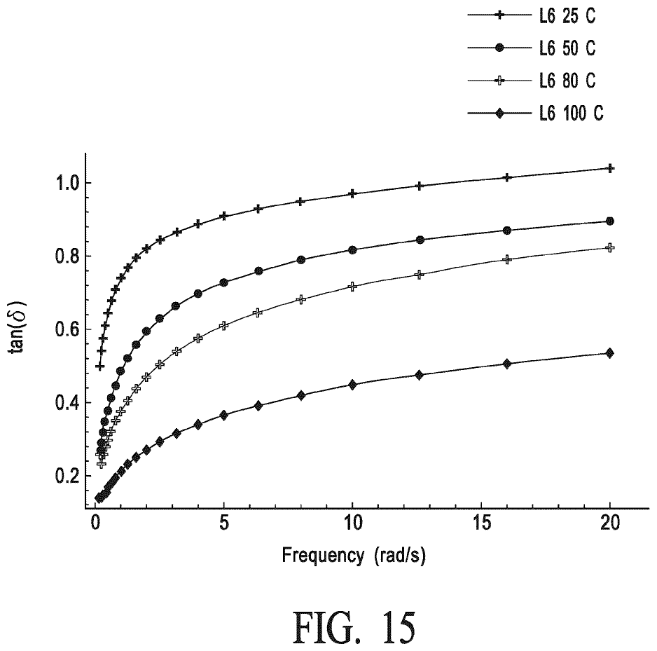

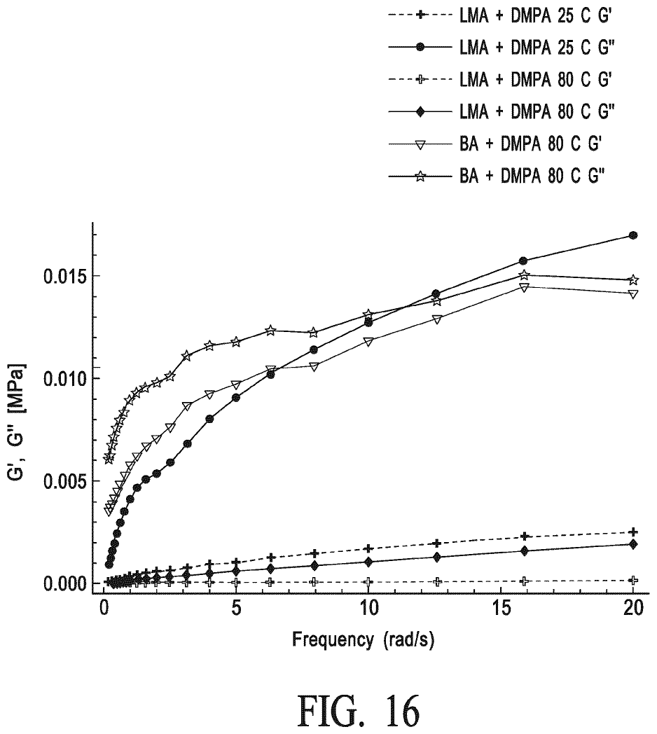

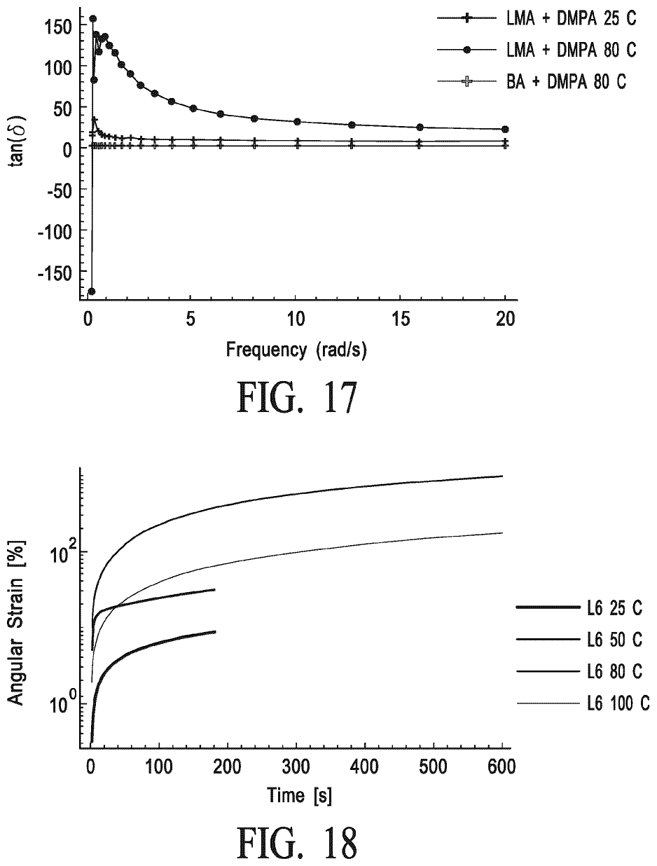

Frequency sweeps were performed on a TA Instruments Discovery HR-2 rheometer to measure the loss and storage moduli. A strain oscillation amplitude sweep was performed on the adhesive sample at 25 °C to determine the nominal oscillating strain amplitude. The storage and loss moduli curves were found to be constant, and a 1% strain was selected. A logarithmic sweep in angular frequency from 0.2 to 20.0 rad/sec was performed at 25 °C. 50 °C, 80 °C and 100 °C. The ratio of the loss modulus over the storage modulus, tan(delta) is reported, showing a high loss behavior (FIGS. 14-17).

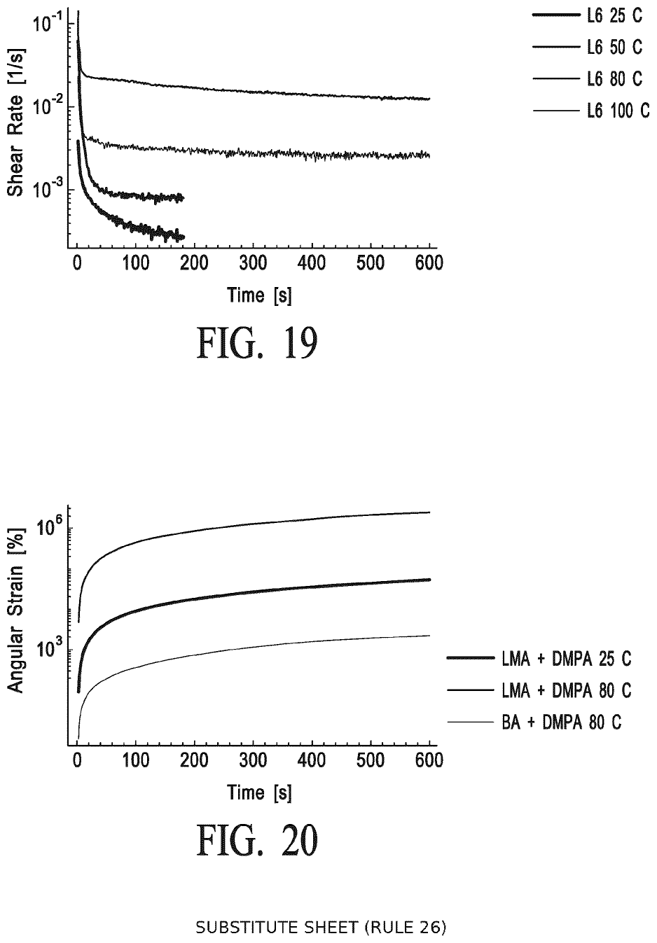

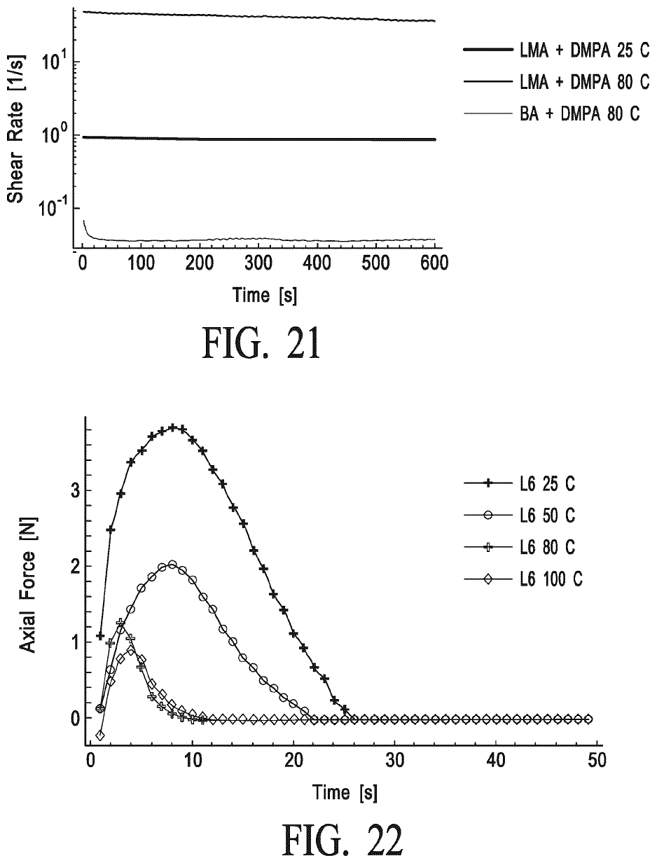

A constant torque of 500 micro-Newton-meters was then applied to samples at 25 °C, 50 °C, 80 °C, and 100 °C, and the resulting shear rate and strain were measured over time, for 180 seconds at 25 °C and 50 °C, and for 600 seconds at 80 °C and 100 °C (FIGS. 18-21).

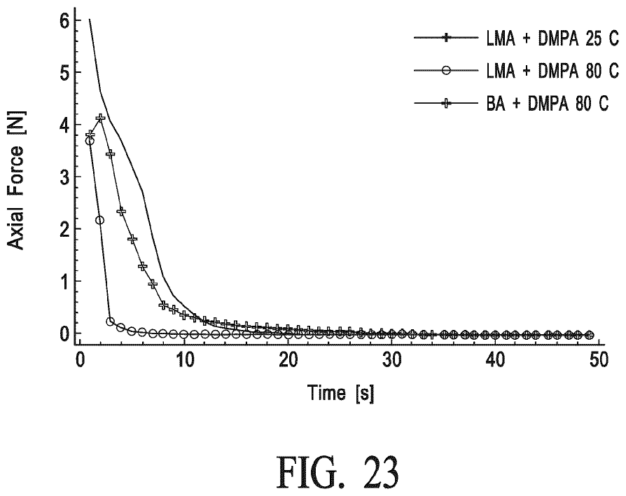

Finally tack strength was measured at 25 °C, 50 °C, 80 °C and 100 °C (FIG. 22 and FIG. 23). The sample was contacted and compressed for 60 seconds, after which the rheometer tip was withdrawn at a rate of 100 micrometers per second, and the axial force was measured. The peak tack force for L6 was found to be 3.8 N at 25 °C, 2.0 N at 50 °C, 1.3 N at 80 °C and 0.9 N at 100 °C. The LMA comparison sample was found to have a peak tack force of 6.0 N at 25 °C and 3.7 N at 80 °C. The BA comparison sample was found to have a peak tack force of 4.1 N at 80 °C.

The same process was repeated for the comparison samples, yielding peak tack forces of 5 N, 6 N, and 4 N for samples (a), (b) and (c) to aluminum, respectively. The adhesive strength, measured in Newton-seconds (Ns), is calculated by integrating the area under the force vs time measurements for each sample, yielding 88.4 Ns for the adhesive, and 9.6 Ns, 5.9 Ns and 3.8 Ns, for comparison samples (a), (b) and (c). The tack energy of the adhesive was also computed by multiplying the adhesive strength integral multiplied by the constant withdraw rate of 100 micrometers/sec, yielding a tack energy of 8.84×10⁻³ Joules (J) for the adhesive, and 0.96×10⁻³ J, 0.59×10⁻³ J and 0.38×10⁻³ J for the comparison samples (a), (b) and (c), respectively.

### 4.8.18. Example 18

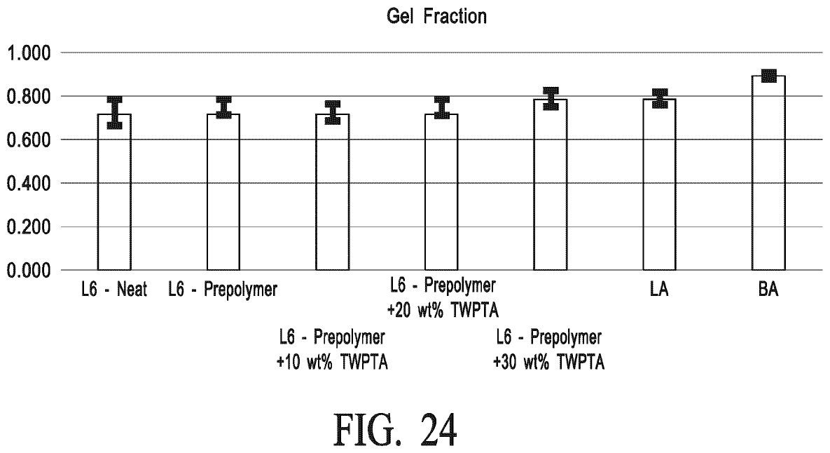

Sol-gel analysis was carried out according to known methods in dichloromethane (DCM) on UV cured samples. Cured polymer samples of masses ranging from approximately 0.2 g to 0.6 g were massed in tared glass vials and subjected to approximately 40 mL of DCM in sealed glass vials. The vials were mixed in a RapidVap vortexer at 35 RPM for 24 hours at approximately 25 °C, after which the DCM was decanted, resulting consistent gels, highly swollen in DCM, and were allowed to dry for 24 hours at ambient temperature and pressure in a commercial chemical fume hood and then further dried for 3 hours at 60 °C and subsequently 2 hours at 120 °C. Final masses of remaining samples/vials were taken, and gel fractions were calculated as final polymer mass divided by initial polymer mass. Representative data are shown in Table 1 and FIG. 24.

TABLE 1

| | Weight | Mole | | Gel |
|---|---|---|---|---|
| | Ratio | Ratio | Gel | Fraction |
| Sample | TMPTA | TMPTA | Fraction | StDev |
| L6- Neat | 0.00600 | 0.006988 | 0.726176221 | 0.062066072 |
| L6- Prepolymer | 0.00600 | 0.006988 | 0.753822567 | 0.034776617 |
| L6- Prepolymer + | 0.00660 | 0.007687 | 0.72430944 | 0.03909231 |
| 10 wt % TMPTA | | | | |
| L6- Prepolymer + | 0.00720 | 0.008386 | 0.751012672 | 0.039157418 |
| 20 wt % TMPTA | | | | |
| L6- Prepolymer + | 0.00780 | 0.009085 | 0.788462401 | 0.039040367 |
| 30 wt % TMPTA | | | | |
| LA | 0.00600 | 0.007396 | 0.794309658 | 0.027018842 |
| BA | 0.00600 | 0.013872 | 0.901328696 | 0.009819267 |

Despite highly weight ratios and mole ratios (within 2x crosslink density), the adhesives exhibited similar gel fraction in the range of 0.70 to 0.90.

### 4.8.19. Example 19

A series of condoms were fabricated using L6, L6+10 wt % increased TMPTA, L6+20 wt % increased TMPTA, L6+30 wt % increased TMPTA, lauryl acrylate (LA) with 0.6 wt % TMPTA and a Styrenic adhesive. Condoms approximately 1 inch by 2 inches rectangular in shape or 1 inch in diameter circular in shape were prepared by transferring approximately 3 inches by 3 inches by squares of adhesive to latex dental dams approximately 0.20 mm in diameter. Adhesive thicknesses ranged from 200 microns to 800 microns thick.

The fabricated condoms were placed on a combination of erect and flaccid penises of several individuals. In some cases, vaginal penetrative sexual intercourse was then carried out, in other cases manual stimulation was carried out. Subsequently, the adhesives were removed from the penis, and the pain self-reported using a standard Wong-Baker type pictogram-numeric pain scale ranging from 0 (no pain) to 10 (worst imaginable pain).

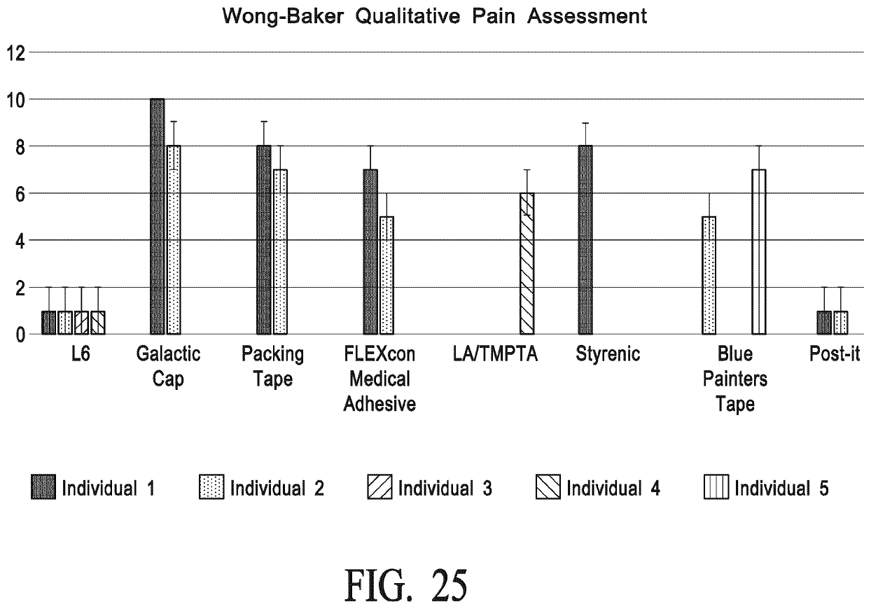

Additionally, commercially available adhesive tapes were tested, which included the Galactic Cap, FLEXcon medical adhesive, 3M Scotch-brand clear packing tape, 3M Scotch-brand blue painters tape and 3M post-it notes. Results are shown in FIG. 25., and the number of samples of each type of adhesive per individual is reported in Table 2.

TABLE 2

| Adhesive | Individual | Individual | Individual | Individual | Individual |
|---|---|---|---|---|---|
| Type | 1 | 2 | 3 | 4 | 5 |
| L6 | >100 | >25 | >5 | 2 | |
| Galactic | 8 | 2 | | | |
| Cap | | | | | |
| Packing | 2 | 6 | | | |
| Tape | | | | | |
| FLEXcon | 5 | 4 | | | |
| Medical | | | | | |
| Adhesive | | | | | |
| LA/TMPTA | | | | 1 | |
| Styrenic | 1 | | | | |
| Blue | | 8 | | | 2 |
| Painters | | | | | |
| Tape | | | | | |
| Post-It | 10 | 10 | | | |

It was observed that L6 and L6 valiant samples typically took between 3 and 15 seconds to remove, averaging between 5 and 8 seconds. Galactic Cap samples took more than 5 minutes to remove per instance and required the use of coconut oil or hydrating lotion. Packing tape, FLEXcon medical adhesive and blue painters' tape all typically took between 10 and 30 seconds to remove, depending on surface area of the sample. The post-it note while relatively pain free and fast to remove (between 1 and 5 seconds), had a relatively low adhesion force.

### 5. EQUIVALENTS AND INCORPORATION BY REFERENCE

While the provided disclosure has been particularly shown and described with reference to a preferred embodiment and various alternate embodiments, it will be understood by persons skilled in the relevant art that various changes in form and details can be made therein without departing from the spirit and scope of the provided disclosure.

All references, issued patents and patent applications cited within the body of the instant specification are hereby incorporated by reference in their entirety, for all purposes. In particular, U.S. Provisional Patent Application Nos. 63/381,071 (filed Oct. 26, 2022); 63/381,653 (filed Oct. 31, 2022); 63/493,761 (Apr. 2, 2023); 63/501,237 (filed May 10, 2023); 63/493,762 (filed Apr. 2, 2023); and 63/501,238 (filed May 10, 2023) are hereby incorporated by reference in their entirety. Additionally, the following PCT patent application, concurrently filed with the present application, is also incorporated by reference in its entirety
the application titled “ENHANCED SENSATION CONDOM” filed October 26. 2023 under attorney docket no. 41822-57380 (001WO).

What is claimed is:

1. A medical adhesive composition comprising a stimuli-responsive polymer formed from one or more monomers and optionally one or more polyfunctional crosslinkers that crosslink the monomers, wherein
the composition is suitable for adhering to a bodily surface; and
the composition delaminates from the bodily surface to which it is adhered following application of a stimulus to the composition.

2. The medical adhesive composition of claim 1, wherein the composition becomes less adhesive or delaminates from the bodily surface to which it is adhered in 0.1 s to 60 s in response to the stimulus, preferably from 2 s to 30 s, more preferably from 1 s to 15 s.

3. The medical adhesive composition of claim 1 or 2, wherein the stimulus is selected from a temperature change, a physico-chemical change, light, ultrasound, an ionic strength change, a pH change, magnetism, mechanical action, and mechanical force.

4. The medical adhesive composition of claim 3, wherein the stimulus is mechanical action.

5. The medical adhesive composition of claim 4, wherein the mechanical force is shear rate.

6. The medical adhesive composition of claim 5, wherein the shear rate is induced by pulling, peeling, or rubbing the composition at varying rates.

7. The medical adhesive composition of claim 3, wherein the stimulus is a temperature change.

8. The medical adhesive composition of claim 7, wherein the composition adheres to the bodily surface at a temperature of 370 C or higher and is less adhesive or delaminates from the bodily surface at a temperature of 25 °C or lower.

9. The medical adhesive composition of claim 3, wherein the stimulus is a physico-chemical change, and the physico-chemical change is dissolution of the composition when contacted by a solvent.

10. The medical adhesive composition of claim 9, wherein the composition adheres to the bodily surface in the absence of the solvent and is less adhesive or delaminates from the surface when contacted by the solvent.

11. The medical adhesive composition of any one of claims 1-10, wherein the bodily surface is human skin.

12. The medical adhesive composition of any one of claims 1-11, wherein the bodily surface is the skin of a human subject, and wherein following adhesion of the composition to the bodily surface, removal of the composition from the bodily surface by light peeling provides minimal or no pain as measured by WBQPA, e.g., a WBQPA score of less than 4, less than 3, less than 2, less than 1, or 0.

13. The medical adhesive composition of any one of claims 1-12, wherein the stimuli-responsive polymer has a lower peel strength at lower peel rates and a higher peel strength at higher peel rates.

14. The medical adhesive composition of any one of claims 1-13, wherein the stimuli-responsive polymer has at least a 5% lower peel strength at a lower peel rate compared to the peel strength at a higher peel rate, e.g., at least 10% lower, at least 20% lower, at least 30% lower, at least 40% lower, at least 50% lower, at least 60% lower, at least 70% lower, at least 80% lower, at least 90% lower, or at least 100% lower.

15. The medical adhesive composition of any one of claims 1-14, wherein the stimuli-responsive polymer has a lower peel strength at a peel rate of 100 mm/min than at 200 mm/min.

16. The medical adhesive composition of any one of claims 1-15, wherein the stimuli-responsive polymer has a lower peel strength at a peel rate of 100 mm/min than at 300 mm/min.

17. The medical adhesive composition of any one of claims 1-16, wherein the stimuli-responsive polymer has a peel strength from 1 to 400 N/m at a peel rate of 100 mm/min, preferably from 1 to 300 N/m, more preferably from 1 to 200 N/m, still more preferably from 1 to 100 N/m, wherein the peel strength is determined by a 180° peel test using a human skin substrate analog.

18. The medical adhesive composition of any one of claims 1-17, wherein the stimuli-responsive polymer has (a) a tack strength at 25 °C of at least 1 N, e.g., from 1 N to 5 N, or (b) an adhesive strength at 25 °C at least 20 N*s, e.g., at least 50 N*s, or at least 100 N*s r.

19. The medical adhesive composition of any one of claims 1-18, wherein the stimuli-responsive polymer has less tack when wet compared to when dry.

20. The medical adhesive composition of any one of claims 1-19, wherein the stimuli-responsive polymer has a storage modulus of from 0.01 MPa to 1 MPa, e.g., from 0.1 MPa to 1 MPa, from 0.1 MPa to 0.8 MPa, or from 0.1 MPa to 0.5 MPa.

21. The medical adhesive composition of any one of claims 1-20, wherein the stimuli-responsive polymer has a loss modulus from 0.1 MPa to 1 MPa, preferably from 0.1 MPa to 0.8 MPa, more preferably from 0.1 MPa to 0.5 MPa

22. The medical adhesive composition of any one of claims 1-21, wherein the stimuli-responsive polymer has a tan(δ) (ratio of storage modulus (G″) to loss modulus (G′) of 0.1 to 2, e.g., 0.1 to 1.5, 0.1 to 1, 0.1 to 0.5, 0.3 to 1, or 0.5 to 1.

23. The medical adhesive composition of any one of claims 1-22, wherein the composition is in the form of a membrane having a thickness from 25 μm to 2,000 μm, e.g., from 25 μm to 1,500 m, from 25 m to 1,000 m, from 25 μm to 500 μm, from 25 μm to 250 μm, or from 25 μm to 100 μm.

24. The medical adhesive composition of claim 23, wherein the membrane has a planar or curved geometry selected from a square, circle, oval, hemisphere, rectangle, polygon, or curvilinear polygon.

25. The medical adhesive composition of claim 23, wherein the membrane has a circle geometry.

26. The medical adhesive composition of claim 25, wherein the radius of the circle is at least about 0.5 cm, e.g., about 1.0 cm, about 2.0 cm, about 3.0 cm, or about 5.0 cm.

27. The medical adhesive composition of claim 23, wherein the membrane has a rectangle geometry.

28. The medical adhesive composition of claim 27, wherein the rectangle has a length from 0.5 cm to 5 cm and a width from 0.5 cm to 5 cm.

29. The medical adhesive composition of any one of claims 1-28, wherein the composition is transparent.

30. The medical adhesive composition of any one of claims 1-29, wherein the stimuli-responsive polymer has a glass transition temperature (Tg) from 0 °C to 50 °C, preferably from 0 °C to 40 °C, more preferably from 5 °C to 40 °C.

31. The medical adhesive composition of any one of claims 1-30, wherein the stimuli-responsive polymer is a crosslinked polymer.

32. The medical adhesive composition of claim 31, wherein the crosslinked polymer has a heterogeneous crosslink density.

33. The medical adhesive composition of claim 31 or 32, wherein the crosslinked polymer comprises C6 to C30 side chains or C6 to C30 dangling chain ends.

34. The medical adhesive composition of claim 33, wherein the C6 to C30 side chains or C6 to C30 dangling chain ends are C6 to C30 alkyl side chains, preferably C12 to C18 alkyl side chains, or C6 to C30 dangling chain ends, preferably C12 to C18 dangling chain ends.

35. The medical adhesive composition of any one of claims 31-34, wherein the crosslinked polymer is a semi-interpenetrating network or the crosslinked copolymer is an interpenetrating network.

36. The medical adhesive composition of any one of claims 1-35, wherein the stimuli-responsive polymer comprises monomers selected from acrylate monomers, methacrylate monomers, vinyl ether monomer, allyl monomers, thiol monomers, epoxy monomers, amine monomers, electron rich monomers, electron poor monomers, lactam monomers, lactone monomers, alcohol monomers, carboxylic acid monomers, isocyanate monomers, Diels-Alder monomers, ring opening metathesis monomers, or a combination thereof.

37. The medical adhesive composition of claim 36, wherein the stimuli-responsive polymer comprises acrylate monomers.

38. The medical adhesive composition of claim 37, wherein the acrylate monomers are C6-C30 alkyl acrylate monomers, preferably C8-C30 alkyl acrylate monomers, preferably C8-C20 alkyl acrylate monomers, more preferably C8-C16 alkyl acrylate monomers.

39. The medical adhesive composition of claim 38, wherein the C8-C30 alkyl acrylate monomers are selected from octyl acrylate, nonyl acrylate, decyl acrylate, undecyl acrylate, dodecyl acrylate, tridecyl acrylate, tetradecyl acrylate, pentadecyl acrylate, hexadecyl acrylate, heptadecyl acrylate, octadecyl acrylate, nonadecyl acrylate, eicosyl acrylate, heneicosyl acrylate, docosyl acrylate, tricosyl acrylate, tetracosyl acrylate, pentacosyl acrylate, hexacosyl acrylate, heptacosyl acrylate, octacosyl acrylate, nonacosyl acrylate, triacontyl acrylate, and combinations thereof.

40. The medical adhesive composition of claim 37, wherein the acrylate monomers are selected from methyl acrylate, ethyl acrylate, Butyl acrylate, 2-ethylhexyl acrylate, isobutyl acrylate, Methoxyethyl acrylate, Hydroxyethyl acrylate, Hydroxypropyl acrylate, Ethoxylated hydroxyethyl acrylate, Glycidyl acrylate, Methacrylic acid, Methyl methacrylate, Ethyl methacrylate, Butyl methacrylate, 2-Hydroxyethyl methacrylate, Cyclohexyl methacrylate, Poly(ethylene glycol) methacrylate, Poly(ethylene glycol) diacrylate, Poly(ethylene glycol) dimethacrylate, Trimethylolpropane triacrylate, Triethylene glycol diacrylate, Tetraethylene glycol diacrylate, Neopentyl glycol diacrylate, Diethylene glycol diacrylate, Dipentaerythritol hexaacrylate, Ethoxylated trimethylolpropane triacrylate, Propoxylated glycerol triacrylate, Stearyl acrylate, Lauryl acrylate, Isodecyl acrylate, Acrylic acid, Ethylene glycol diacrylate (EGDA), Triethylene glycol diacrylate (TEGDA), Propylene glycol diacrylate (PGDA), Butanediol diacrylate (BDDA), Neopentyl glycol diacrylate (NPGDA), Pentaerythritol tetraacrylate (PETA), 1,4-Butanediol diacrylate (BDA), Di(trimethylolpropane) tetraacrylate (DTMPTA), Bisphenol A ethoxylate diacrylate (BPAEDA), Ethoxylated bisphenol A diacrylate (EBPA), Decanediol diacrylate, Polyethylene glycol diacrylate (PEGDA), Trimethylolpropane triacrylate (TMPTA), Diethylene glycol diacrylate (DEGDA), and 1,6-Hexanediol diacrylate (HDDA), Trimethylolpropane triacrylate (TMPTA), Tripropylene glycol diacrylate (TPGDA), Pentaerythritol triacrylate (PETA), Dipentaerythritol pentaacrylate (DPEPA), Tris(2-hydroxyethyl) isocyanurate triacrylate (THEIC-TA), Triethylene glycol dimethacrylate (TEGDMA), Triallyl isocyanurate (TAIC), Triethylene glycol diacrylate (TEGDA), Ethoxylated trimethylolpropane triacrylate (ETMPTA), Triallyl cyanurate (TAC), and combinations thereof.

41. The medical adhesive composition of any one of claims 37-40, wherein the stimuli-responsive polymer comprises at least 10 wt % acrylate monomers, e.g., at least 50 wt %, at least 65 wt %, at least 95 wt %, at least 96 wt %, at least 97 wt %, at least 98 wt %, or at least 99 wt %.

42. The medical adhesive composition of claim 36, wherein the stimuli-responsive polymer comprises methacrylate monomers.

43. The medical adhesive composition of claim 42, wherein the methacrylate monomers are C6-C30 alkyl methacrylate monomers, preferably C8-C30 alkyl methacrylate monomers, preferably C8-C20 alkyl methacrylate monomers, more preferably C8-C16 alkyl methacrylate monomers.

44. The medical adhesive composition of claim 43, wherein the C8-C30 alkyl methacrylate monomers are selected from octyl methacrylate, nonyl methacrylate, decyl methacrylate, undecyl methacrylate, dodecyl methacrylate, tridecyl methacrylate, tetradecyl methacrylate, pentadecyl methacrylate, hexadecyl methacrylate, heptadecyl methacrylate, octadecyl methacrylate, nonadecyl methacrylate, eicosyl methacrylate, heneicosyl methacrylate, docosyl methacrylate, tricosyl methacrylate, tetracosyl methacrylate, pentacosyl methacrylate, hexacosyl methacrylate, heptacosyl methacrylate, octacosyl methacrylate, nonacosyl methacrylate, triacontyl methacrylate, and combinations thereof.

45. The medical adhesive composition of claim 42, wherein the methacrylate monomers are selected from Ethyl methacrylate, Butyl methacrylate, 2-Hydroxyethyl methacrylate, Cyclohexyl methacrylate, Isobornyl methacrylate, Stearyl methacrylate, Lauryl methacrylate, Isodecyl methacrylate, Tetrahydrofurfuryl methacrylate, Glyceryl methacrylate, Trimethylolpropane trimethacrylate, Trimethylolpropane triacrylate, Pentaerythritol triacrylate, Pentaerythritol tetramethacrylate, Poly(ethylene glycol) monomethyl ether methacrylate, Poly(ethylene glycol) monomethyl ether acrylate, Poly(ethylene glycol) diacrylate, Poly(ethylene glycol) dimethacrylate, Poly(ethylene glycol) monoacrylate, Ethoxylated bisphenol A dimethacrylate, Ethoxylated bisphenol A diacrylate, Ethoxylated trimethylolpropane triacrylate, Hydroxypropyl methacrylate, Methacrylic acid, Acryloyloxyethyltrimethylammonium chloride, Diethylaminoethyl methacrylate, Butylaminoethyl methacrylate, N,N-dimethylaminoethyl methacrylate, Methacryloyloxyethyl phthalate, Cyclopropyl methacrylate, and combinations thereof.

46. The medical adhesive composition of any one of claims 42-45, wherein the stimuli-responsive polymer comprises poly (lauryl methacrylate).

47. The medical adhesive composition of any one of claims 42-46, wherein the stimuli-responsive polymer comprises at least 10 wt % methacrylate monomers, e.g., at least 50 wt %, at least 65 wt %, at least 95 wt %, at least 96 wt %, at least 97 wt %, at least 98 wt %, or at least 99 wt %.

48. The medical adhesive composition of claim 36, wherein the stimuli-responsive polymer comprises vinyl ether monomers selected from divinyl ether of ethylene glycol, divinyl ether of diethylene glycol, divinyl ether of triethylene glycol, divinyl ether of polyethylene glycol (DVE-PEG), divinyl ether of polypropylene glycol (DVE-PPG), Divinyl ether of poly(ethylene glycol) methyl ether (DVE-PEGME), divinyl ether of poly(ethylene glycol) butyl ether (DVE), Divinyl ether of poly(ethylene glycol) phenyl ether (DVE-PEGPhE), Divinyl ether of glycerol (DVE-Gly), Divinyl ether of 1,4-cyclohexanedimethanol (DVE-CHDM), Divinyl ether of neopentyl glycol (DVE-NPG), and combinations thereof.

49. The medical adhesive composition of claim 36, wherein the stimuli-responsive polymer comprises allyl monomers selected from Diallyl phthalate (DAP), Diallyl maleate (DAM), Diallyl succinate (DAS), Diallyl fumarate (DAF), Diallyl adipate (DAA), Diallyl sebacate (DAS), Diallyl terephthalate (DAT), Diallyl isophthalate (DAI), Diallyl itaconate (DAI), Diallyl carbonate (DAC), Diallyl diglycolate (DADG), Diallyl tris(2-hydroxyethyl) isocyanurate (DATHEIC), Triallyl cyanurate (TAC), Triallyl isocyanurate (TAIC), Triallyl trimellitate (TATM), Triallyl citrate (TAC), Triallyl phosphate (TAP), Triallylamine (TAA), Triallyl cyanide (TACN), Triallyl benzene-1,2,4-tricarboxylate (TABTC), Triallyl trimesate (TATM), Tris(2-hydroxyethyl) isocyanurate triallyl ether (THEIC-TAE), and combinations thereof.

50. The medical adhesive composition of claim 36, wherein the stimuli-responsive polymer comprises thiol monomers selected from 3-Mercaptopropionic acid; Thioglycolic acid; 3-Mercapto-1-propanol; 2-Mercaptoethanol; 2-(2-Mercaptoethoxy)ethanol; 2-(2-Mercaptopropionylamino)ethanol; 2-(2-Mercaptosuccinyl)ethyl acrylate; 3-(2-Mercaptopropionylamino)propionic acid; 3-(Mercaptopropyl)trimethoxysilane; 2,2′-(Ethylenebis(thio))diethanol; 3-Mercaptopropyltrimethoxysilane; 3-Mercaptopropylmethyldimethoxysilane; 3-(2,2-Dithiobis(ethylthio)propionylamino)propionic acid; 3,6,9-Trioxadecanethiol; 3-Mercapto-1,2-propanediol; 2,2′-Dithiodiethanol; N-Acetyl-L-cysteine; L-Cysteine; 2-(2-Mercaptoethyl)pyridine; 4-(2-Mercaptoethyl)morpholine; 3-Mercapto-1,2,4-triazole; Thiophenol; Pentaerythritol tetrakis(3-mercaptopropionate) (PETMP); Trimethylolpropane tris(3-mercaptopropionate) (TMPMP); Triethanolamine tris(3-mercaptopropionate) (TEAMP); Tris(2-hydroxyethyl) isocyanurate tris(3-mercaptopropionate) (THEICMP); Bis(3-mercaptopropyl) sulfide (BMPS); 1,2-ethanedithiol (EDT); 1,3-propanedithiol; 1,4-butanedithiol; 1,6-hexanedithiol; 1,8-octanedithiol and combinations thereof.

51. The medical adhesive composition of claim 36, wherein the stimuli-responsive polymer comprises epoxy monomers selected from Bisphenol A diglycidyl ether (BADGE), Bisphenol F diglycidyl ether (BFDGE), Novolac diglycidyl ether (NGDE), Phenol novolac diglycidyl ether (PNGDE), Cycloaliphatic epoxy resins, Glycidyl ethers of aliphatic alcohols, Glycidyl ethers of aromatic alcohols, Triglycidyl isocyanurate (TGIC), Diglycidyl ether of 1,4-butanediol (BDDGE), Diglycidyl ether of neopentyl glycol (NPGDGE), Diglycidyl ether of propylene glycol (PGDGE), Epoxidized soybean oil (ESO), Epoxidized linseed oil (ELO), Dicyclopentadiene-based epoxy resins, Tetrafunctional epoxy resins, Epoxy phenolic novolac resins, and combinations thereof.

52. The medical adhesive composition of claim 36, wherein the stimuli-responsive polymer comprises amine monomers selected from Ethylenediamine, Diethylenetriamine, Triethylenetetramine, Tetraethylenepentamine, Polyethyleneimine, Diaminopropane, Diaminobutane, Diaminopentane, Diethylenetriaminepentaacetic acid (DTPA), Tris(2-aminoethyl)amine, N-(2-Aminoethyl)piperazine, N-(3-Aminopropyl)morpholine, N,N-Dimethylaminopropylamine, N,N-Dimethylethylenediamine, 1,3-Diaminopropane, Isophoronediamine, Jeffamine D-230, Jeffamine T-403, Jeffamine M-207, Jeffamine EDR-148, and combinations thereof.

53. The medical adhesive composition of claim 36, wherein the stimuli-responsive polymer comprises electron rich monomers selected from Vinyl ethers (e.g. vinyl methyl ether, vinyl ethyl ether), Vinyl acetate, Allyl alcohol, Allyl amine, N-Methylolacrylamide, N-Methylolmethacrylamide, N-Methylolallylamine, N-Methylolvinylacetamide, Acrolein diethyl acetal, Acrolein diethyl ketal, Diacetone acrylamide, 2-Hydroxyethyl acrylate, 2-Hydroxyethyl methacrylate, 2-Hydroxypropyl acrylate, 2-Hydroxypropyl methacrylate, 2,3-Dihydroxypropyl methacrylate, Glycidyl methacrylate, Glycidyl acrylate, Tetrahydrofurfuryl methacrylate, and N-Vinylpyrrolidone, n-vinylformamide, n-vinyl pyridine, styrene, styrene derivatives, and combinations thereof.

54. The medical adhesive composition of claim 36, wherein the stimuli-responsive polymer comprises electron poor monomers selected from Acrylonitrile, Methacrylonitrile, Methyl methacrylate, Acrylic acid, Methacrylic acid, Maleic anhydride, Itaconic acid, Fumaric acid, Acrylamide, Methacrylamide, N-Vinylcarbazole, Vinylidene chloride, Vinyl chloride, Vinyl sulfonic acid, Vinyl acetate, Styrene, Alpha-,methylstyrene, Maleimide, N-Phenylmaleimide, and N-Butylmaleimide, maleic anhydride, and combinations thereof.

55. The medical adhesive composition of claim 36, wherein the stimuli-responsive polymer comprises lactam monomers selected from Caprolactam, Valerolactam, Enantholactam, Capryllactam, Laurinlactam, Prolactam, Butyrolactam, Methionyl lactam, Methoxyethyl lactam, Methoxyethyl methionyl lactam, Dimethylaminoethyl lactam, Dimethylaminoethyl methionyl lactam, Dimethylaminoethyl acryloyl lactam, Dimethylaminoethyl methacryloyl lactam, N-vinylpyrrolidone, N-methylpyrrolidone, N-ethylpyrrolidone, and combinations thereof.

56. The medical adhesive composition of claim 36, wherein the stimuli-responsive polymer comprises lactone monomers selected from β-propiolactone, γ-butyrolactone, 8-valerolactone, ε-caprolactone, ω-pentadecalactone, β-butyrolactone, 8-decalactone, ε-decalactone, γ-decalactone, 8-dodecalactone, γ-dodecalactone, α-methylene-γ-butyrolactone, β-methyl-y-butyrolactone, β-methyl-γ-valerolactone, γ-hexalactone, and combinations thereof.

57. The medical adhesive composition of claim 36, wherein the stimuli-responsive polymer comprises alcohol monomers selected from Ethylene glycol, Propylene glycol, 1,3-Butanediol, 1,4-Butanediol, 1,5-Pentanediol, 1,6-Hexanediol, 1,10-Decanediol, Neopentyl glycol, Diethylene glycol, Triethylene glycol, Tetraethylene glycol, Polyethylene glycol (PEG), Polypropylene glycol (PPG), Polycaprolactone diol, Polymethylolpropane, Hydroxypivalyl hydroxymethylbutyrate (HPHMB), 1,4-Cyclohexanedimethanol, and combinations thereof.

58. The medical adhesive composition of claim 36, wherein the stimuli-responsive polymer comprises carboxylic acid monomers selected from Adipic acid, Succinic acid, Glutaric acid, Sebacic acid, Malonic acid, Phthalic acid, Isophthalic acid, Terephthalic acid, Fumaric acid, Maleic acid, Itaconic acid, Citric acid, 1,4-Cyclohexanedicarboxylic acid, 1,3-Cyclohexanedicarboxylic acid, Dodecanedioic acid, and combinations thereof.

59. The medical adhesive composition of claim 36, wherein the stimuli-responsive polymer comprises isocyanate monomers selected from Toluene diisocyanate (TDI), Diphenylmethane diisocyanate (MDI), Hexamethylene diisocyanate (HDI), Isophorone diisocyanate (IPDI), 1,6-Hexamethylene diisocyanate (HMDI), 4,4′-Methylenebis(cyclohexyl isocyanate) (H12MDI), Naphthalene diisocyanate (NDI), 2,4-Toluene diisocyanate (2,4-TDI), 2,6-Toluene diisocyanate (2,6-TDI), Polymethylene polyphenyl isocyanate (PAPI), Desmodur N-100, Desmodur L-75, Desmodur HL, Desmodur H, Desmodur VP, Desmodur Z, and combinations thereof.

60. The medical adhesive composition of claim 36, wherein the stimuli-responsive polymer comprises Diels-Alder monomers selected from Maleic anhydride, Furan, Cyclopentadiene, N-phenylmaleimide, Anthracene, N-ethylmaleimide, N-phenylnorbornene, N,N-dimethyl maleimide, 2,5-dimethylfuran, Tetracyanoethylene, Methyl vinyl ketone, and combinations thereof.

61. The medical adhesive composition of claim 36, wherein the stimuli-responsive polymer comprises ring opening metathesis monomers selected from Norbornene, Dicyclopentadiene (DCPD), Cyclooctene, Tetracyclododecene (TCD), Cyclopentene, Cycloheptene, Cyclohexene, Bicyclo[2.2.1]hept-2-ene, Bicyclo[2.2.2]oct-5-ene, Tricyclo[5.2.1.0(2,6)]dec-8-ene (TCD-Diene), and combinations thereof.

62. The medical adhesive composition of any one of claims 1-61, wherein the stimuli-responsive polymer further comprises a polyfunctional crosslinker.

63. The medical adhesive composition of claim 62, wherein the polyfunctional crosslinker is selected from a difunctional crosslinker, a trifunctional crosslinker, or a tetrafunctional crosslinker.

64. The medical adhesive composition of claim 62 or 63, wherein the polyfunctional crosslinker is a trifunctional crosslinker.

65. The medical adhesive composition of claim 64, wherein the trifunctional crosslinker is an acrylate crosslinker.

66. The medical adhesive composition of claim 62, wherein the polyfunctional crosslinker is selected from poly(ethylene glycol) diacrylate, trimethylolpropane triacrylate; ethoxylated trimethyolpropane triacrylate; pentaerythritol tetraacrylate; ethoxylated pentaerythritol tetraacrylate; dipentaerithrotol hexaacrylate; ethoxylated dipentaerithrotol hexaacrylate; di-, tri-, tetra-, penta-, or hexa-epoxides; polythiols; polyalkenes; tris(2-acryloxyethyl) isocyanulate, ε-caprolactone modified tris(2-acryloxyethyl) isocyanurate, ethoxylated glycerine triacrylate, ethoxylated glycerine triacrylate, pentaerythritol triacrylate, and combinations thereof.

67. The medical adhesive composition of any one of claims 1-66, wherein the stimuli-responsive polymer comprises from 0.1 wt % to 1.5 wt % polyfunctional crosslinker, preferably from 0.2 wt % to 1 wt %, more preferably from 0.4 wt % to 0.8 wt %.

68. The medical adhesive composition of any one of claims 62-67, wherein the weight ratio of the one or more monomers to the one or more polyfunctional crosslinkers is from 98:2 to 99.9:0.1, e.g., from 98.5:1.5 to 99.9:0.1; from 99:1 to 99.9:0.1, from 99.1:0.9 to 99.9:0.1, from 99.2:
0.8 to 99.9:0.1; from 99.3:0.7 to 99.9:0.1, from 99.4:0.6 to 99.9:0.1, from 99.5:0.5 to 99.9:0.1, from 99.6:0.4 to 99.9:0.1, from 99.7:0.3 to 99.9:0.1, from 99.8:0.2 to 99.9:0.1.

69. The medical adhesive composition of claim 1, wherein the stimuli-responsive polymer comprises a poly(lauryl methacrylate) polymer crosslinked with the one or more polyfunctional crosslinkers.

70. The medical adhesive composition of claim 69, wherein the polyfunctional crosslinker is selected from a difunctional crosslinker, a trifunctional crosslinker, or a tetrafunctional crosslinker.

71. The medical adhesive composition of claim 69 or 70, wherein the polyfunctional crosslinker is a trifunctional crosslinker.

72. The medical adhesive composition of claim 71, wherein the trifunctional crosslinker is an acrylate crosslinker.

73. The medical adhesive composition of claim 69, wherein the polyfunctional crosslinker is selected from poly(ethylene glycol) diacrylate, trimethylolpropane triacrylate; ethoxylated trimethyolpropane triacrylate; pentaerythritol tetraacrylate; ethoxylated pentaerythritol tetraacrylate; dipentaerithrotol hexaacrylate; ethoxylated dipentaerithrotol hexaacrylate; di-, tri-, tetra-, penta-, or hexa-epoxides; polythiols; polyalkenes; tris(2-acryloxyethyl) isocyanulate, F-caprolactone modified tris(2-acryloxyethyl) isocyanurate, ethoxylated glycerine triacrylate, ethoxylated glycerine triacrylate, pentaerythritol triacrylate, and combinations thereof.

74. The medical adhesive composition of any one of claims 69-73, wherein the weight ratio of lauryl methacrylate to the one or more polyfunctional crosslinkers is from 98:2 to 99.9:0.1, e.g., from 98.5:1.5 to 99.9:0.1; from 99:1 to 99.9:0.1, from 99.1:0.9 to 99.9:0.1, from 99.2:0.8 to 99.9:0.1; from 99.3:0.7 to 99.9:0.1, from 99.4:0.6 to 99.9:0.1, from 99.5:0.5 to 99.9:0.1, from 99.6:0.4 to 99.9:0.1, from 99.7:0.3 to 99.9:0.1, from 99.8:0.2 to 99.9:0.1.

75. The medical adhesive composition of any one of claims 1-74, wherein the composition further comprises an additive selected from tackifiers, plasticizers, pigments, fillers, fluorescents, flow agents, wetting agents, surfactants, anti-foaming agents, rheology modifiers, colorants, permeation enhancers, stabilizers, antioxidants, or combinations thereof.

76. An article comprising the medical adhesive composition of any one of claims 1-75 adhered to a substrate, thereby providing an adhesive surface.

77. The article of claim 76, wherein the substrate is a backing layer or release liner.

78. The article of 77, wherein the substrate is a backing layer, and the backing layer comprises non-woven fabrics, woven fabrics, films (e.g., sheets), paper, composite materials obtained by laminating a film on a non-woven fabric or a woven fabric, or a combination thereof.

79. The article of claim 76, wherein the substrate is a backing layer selected from a polyurethane, poly(vinyl chloride), and latex.

80. The article of any one of claims 76-79, wherein the substrate is elastomeric.

81. The article of any one of claims 76-79, wherein the substrate is rigid.

82. The article of any one of claims 76-81, wherein the article has a planar or curved geometry selected from a square, circle, oval, hemisphere, rectangle, polygon, or curvilinear polygon.

83. The article of claim 82, wherein the article has a circle geometry.

84. The article of claim 83, wherein the radius of the circle is at least about 0.5 cm, e.g., about 1.0 cm, about 2.0 cm, about 3.0 cm, or about 5.0 cm.

85. The article of claim 82, wherein the article has a rectangle geometry.

86. The article of claim 85, wherein the rectangle has a length from 0.5 cm to 5 cm and a width from 0.5 cm to 5 cm.

87. The article of any one of claims 76-86, further comprising a release liner adhered to the adhesive surface of the medical adhesive composition opposite the backing layer.

88. The article of any one of claims 76-87, wherein the article is selected from an adhesive patch, a skin closure strip, a bandage, medical tape, sports tape, a wound dressing, a surgical incision drape, a medical device, or an eye cover.

89. The article of claim 88, wherein the article is a medical device selected from a catheter, port, pump, probe, electrode, stent, tube, and needle.

90. The article of claim 88, wherein the article is a wound dressing, and the article further comprises an absorbent layer.

91. The article of claim 90, wherein when the article is applied to the skin surface of a subject, removal causes minimal or no pain as measured by the WBQPA, e.g., a WBQPA score of less than 4, less than 3, less than 2, less than 1, or 0.

92. The article of any one of claims 76-91, wherein when the adhesive surface of the article is applied to the skin surface of a subject, removal of the article by light peeling results in less than 50 wt % of the adhesive remaining on the skin surface of the subject, e.g., less than 40 wt %, less than 30 wt %, less than 20 wt %, or less than 10 wt %.

93. A package comprising the medical adhesive composition of any one of claims 1-75 or an article of any one of claims 76-92.

94. The package of claim 93, wherein the package comprises a flexible wrapper comprising foil, plastic, plastic-lined paper, foil-lined paper, or a combination thereof.

95. The package of claim 93 or 94, wherein the package is selected from a blister-pack design and a well design.

96. The package of any one of claims 93-95, further comprising a second composition suitable to induce delamination of the medical adhesive composition or article from a bodily surface to which the medical adhesive composition or article is adhered.

97. The package of claim 96, wherein the second composition is a wipe.

98. The package of claim 97, wherein the wipe comprises a solvent that dissolves, denatures, or swells the medical adhesive composition, thereby inducing delamination of the medical adhesive composition from the bodily surface when the wipe is applied to the medical adhesive composition or article.

99. The package of claim 97, wherein the wipe comprises a volatile additive that cools the wipe upon evaporation, thereby inducing delamination of the medical adhesive composition from the bodily surface when the wipe is applied to the medical adhesive composition or article.

100. A kit comprising (a) the medical adhesive composition of any one of claims 1-75 or an article of any one of claims 76-92 and (b) instructions for use.

101. A method of closing a wound comprising:
applying the medical adhesive composition of any of claims 1-75 to a wound having a first surface and a second surface separated by a wound cavity, wherein the composition is applied into the wound cavity;
drawing the first surface and second surfaces of the wound together; and
applying pressure sufficient to close the wound, whereby the composition adheres the first surface and second surface together.

102. A method of closing a wound comprising a first surface and a second surface separated by a wound cavity, the method comprising:
drawing the first surface and second surface together to form a narrowed wound cavity,
applying the medical adhesive composition of any of claims 1-75 to the narrowed wound cavity, and
applying pressure sufficient to close the wound, whereby the composition adheres the first surface and second surface together.

103. The method of claim 102, wherein the first surface and the second surface are drawn together such that the first surface and the second surface are in contact and form a junction, and the method comprises applying the medical adhesive composition superficially to the junction.

104. The method of any one of claims 101-103, wherein the first surface and the second surface are each independently selected from skin, mucosal tissue, connective tissue, epithelial tissue, muscle tissue, nervous tissue, cartilage, tendon, and bone.

105. The method of claim 104, wherein the first surface and the second surface are skin.

106. The method of any one of claims 101-105, wherein the wound is selected from a surgical wound, a laceration, an abrasion, a puncture, a traumatic wound, a diabetic wound, a pressure wound, a burn wound, a chronic wound, and combinations thereof.

107. A method of applying an adhesive article to a subject comprising:
applying the adhesive surface of an article of any one of claims 76-92 to a wound or bodily surface of a subject.

108. The method of claim 107, further comprising removing the adhesive article after at least 1 second, e.g., 1 hour, 12 hours, 24 hours, 1 day, 1 or 1 week, wherein removal comprises application of a stimulus to the article and (a) does not cause pain in the subject as measured by the Numeric Pain Rating System (NPRS), e.g., a NRPS score of less than 4, less than 3, less than 2, less than 1, or 0 or (b) result in medical adhesive-related skin injury (MARSI).

109. The method of claim 107 or 108, wherein the subject has a history of MARSI.

110. The method of claim 108 or 109, wherein the MARSI is selected from mechanical injury, irritation, inflammation, skin stripping, blister, skin tear, irritant contact dermatitis, allergic dermatitis, and maceration.

111. The method of any one of claims 107-110, wherein the subject has sensitive skin.

112. The method of any one of claims 107-111, wherein the subject is less than three months old or greater than 65 years old.

113. The method of any one of claims 107-112, wherein the subject has an underlying health condition.

114. The method of claim 113, wherein the underlying health condition is selected from diabetes, renal insufficiency, immunosuppression, venous insufficiency, malnutrition, dehydration, and antibiotic use.

115. The method of any one of claims 107-114, wherein the bodily surface is selected from the skin of the head, neck, chest, upper limb, lower limb, hand, foot, abdomen, torso, back, buttocks, and genitals.

116. The method of any one of claims 107-115, wherein the adhesive article is applied to a wound, and application of the adhesive article prevents bacterial infection of the wound.

117. The method of any one of claims 107-115, wherein the adhesive article is applied to a wound, and application of the adhesive article forms a seal (i.e., prevents fluid ingress into or egress from the wound).

118. The method of claim 116 or 117, wherein the wound is selected from a surgical wound, a laceration, an abrasion, a puncture, a traumatic wound, a diabetic wound, a pressure wound, a burn wound, a chronic wound, or a combination thereof.

119. The method of claim 108, wherein the stimulus is mechanical action.

120. The method of claim 119, wherein the mechanical action is shear rate.

121. The method of claim 120, wherein shear rate is induced by pulling, peeling, or rubbing at varying rates.

122. The method of claim 108, wherein the stimulus is temperature change, and application of a stimulus comprises cooling the temperature of the adhesive article to 25 °C or lower.

123. The method of claim 108, wherein the stimulus is physico-chemical change, and application of a stimulus comprises applying a wipe to the adhesive article, wherein the wipe comprises a solvent that dissolves, denatures, or swells the stimuli-responsive polymer, thereby inducing delamination of the medical adhesive composition from the wound or bodily surface.

124. The method of claim 123, wherein the wipe comprises a volatile additive that cools the wipe upon evaporation, thereby inducing delamination of the adhesive article from the wound or bodily surface when the wipe is applied to the wound or bodily surface.

125. The method of any one of claims 108-124, wherein when the adhesive article is removed from the wound or bodily surface, less than 50 wt % of the adhesive remains on the wound or bodily surface, e.g., less than 40 wt %, less than 30 wt %, less than 20 wt %, or less than 10 wt %.

126. A method of preparing the medical adhesive composition of any one of claims 1-75, comprising:
(a) curing one or more monomers and a first amount of one or more polyfunctional crosslinkers to form a prepolymer; and
(b) post-curing the prepolymer;
thereby providing the medical adhesive composition.

127. The method of claim 126, wherein the weight ratio of the one or more monomers to the one or more polyfunctional crosslinkers is from 98:2 to 99.9:0.1, e.g., from 98.5:1.5 to 99.9:0.1; from 99:1 to 99.9:0.1, from 99.1:0.9 to 99.9:0.1, from 99.2:0.8 to 99.9:0.1; from 99.3:0.7 to 99.9:0.1, from 99.4:0.6 to 99.9:0.1, from 99.5:0.5 to 99.9:0.1, from 99.6:0.4 to 99.9:0.1, from 99.7:0.3 to 99.9:0.1, from 99.8:0.2 to 99.9:0.1.

128. The method of claim 126 or 127, wherein the curing is a photopolymerization.

129. The method of any one of claims 126-128, wherein the post-curing comprises subjecting the prepolymer to an elevated temperature relative to the curing, e.g., at least 50 °C, at least 100 °C, at least 150 °C, or at least 200 °C.

130. The method of any one of claims 126-129, further comprising pausing curing prior to completion, adding a second amount of one or more polyfunctional crosslinkers, and restarting curing.

131. A method of preparing an article of any one of claims 76-92 comprising:
(a) coating a prepolymer solution onto a substrate to provide a coated substrate, the prepolymer solution comprising one or more monomers and optionally one or more polyfunctional crosslinkers;
(b) curing the coated substrate; and
(c) post-curing the coated substrate
thereby providing the article.

132. The method of claim 131, wherein the weight ratio of the one or more monomers to the one or more polyfunctional crosslinkers is from 98:2 to 99.9:0.1, e.g., from 98.5:1.5 to 99.9:0.1; from 99:1 to 99.9:0.1, from 99.1:0.9 to 99.9:0.1, from 99.2:0.8 to 99.9:0.1; from 99.3:0.7 to 99.9:0.1, from 99.4:0.6 to 99.9:0.1, from 99.5:0.5 to 99.9:0.1, from 99.6:0.4 to 99.9:0.1, from 99.7:0.3 to 99.9:0.1, from 99.8:0.2 to 99.9:0.1.

133. The method of claim 131 or 132, wherein the curing is a photopolymerization.

134. The method of any one of claims 131-133, wherein the post-curing comprises subjecting the coated substrate to an elevated temperature relative to the curing, e.g., at least 50 °C, at least 100 °C, at least 150 °C, or at least 200 °C.

135. The method of any one of claims 131-[0454], further comprising pausing curing prior to completion, adding a second amount of one or more polyfunctional crosslinkers, and restarting curing.
